SEQUENCE LISTING (90 sequences)

SEQ ID NO: 1 - protein, 236 aa, Dasypus novemcinctus
MTSETGPSTGDATLRRRIQPWEFEVFFDPRELRKETCLLYEIKWGRSRKIWRNSGQNTSKHVEVNFIEKLISERHFCPSVSCSITWFLSWSPCWECSKAIREFLSQHPNITLVIYVARLFHHMDQHNRQGLRDLINSGVTIQIMRAEEYDHCWRNFVNYSPGEEAHWPRYPPLWMTMYALELHCIILSLPPCLKITRRCQHQHTFFSLNFQNCHYQMIPPQTLYAAGIIQPSMTWR

SEQ ID NO: 2 - protein, 291 aa, Dasypus novemcinctus
MTSETGPSTGDATLRRRIQPWEFEVFFDPRELAKETCLLYEIKWGRSRKIWRNSGQNTSKHVEVNFIEKLISERHFCPSVSCSITWFLSWSPCWECSKAIREFLSQHPNITLVIYVARLFHHMDQHNRQGLRDLINSGVTIQIMRAEEYDHCWRNFVNYSPGEEAHWPRYPPLWMTMYALELHCIILSLPPCLKITRRCQHQHTFFSLNFQNCHYQMIPPQTLYAAGIIQPSMTWRLHCIILSLPPCLKITRRCQHQHTFFSLNFQNCHYQMIPPQTLYAAGIIQPSMTWR

SEQ ID NO: 3 - protein, 236 aa, Dasypus novemcinctus
MTSETGPSTGDATLRRRIQPWEFEVFFDPRELRAETCLLYEIKWGRSRKIWRNSGQNTSKHVEVNFIEKLISERHFCPSVSCSITWFLSWSPCWECSKAIREFLSQHPNITLVIYVARLFHHMDQHNRQGLRDLINSGVTIQIMRAEEYDHCWRNFVNYSPGEEAHWPRYPPLWMTMYALELHCIILSLPPCLKITRRCQHQHTFFSLNFQNCHYQMIPPQTLYAAGIIQPSMTWR

SEQ ID NO: 4 - protein, 236 aa, Dasypus novemcinctus
MTSETGPSTGDATLRRRIQPWEFEVFFDPRELRKETCLLYEIKWGRSRKIWRNSGQNTSKHVEVNFIEKLISERHFCPSVSCSITWFLSYSPCWECSKAIREFLSQHPNITLVIYVARLFHHMDQHNRQGLRDLINSGVTIQIMRAEEYDHCWRNFVNYSPGEEAHWPRYPPLWMTMYALELHCIILSLPPCLKITRRCQHQHTFFSLNFQNCHYQMIPPQTLYAAGIIQPSMTWR

SEQ ID NO: 5 - protein, 236 aa, Dasypus novemcinctus
MTSETGPSTGDATLRRRIQPWEFEVFFDPRELRKETCLLYEIKWGRSRKIWRNSGQNTSKHVEVNFIEKLISERHFCPSVSCSITWFLSYSPCWECSKAIREFLSQHPNITLVIYVARLFHHMDQENRQGLRDLINSGVTIQIMRAEEYDHCWRNFVNYSPGEEAHWPRYPPLWMTMYALELHCIILSLPPCLKITRRCQHQHTFFSLNFQNCHYQMIPPQTLYAAGIIQPSMTWR

SEQ ID NO: 6 - protein, 236 aa, Dasypus novemcinctus
MTSETGPSTGDATLRRRIQPWEFEVFFDPRELRKETCLLYEIKWGRSRKIWRNSGQNTSKHVEVNFIEKLISERHFCPSVSCSITWFLSWSPCWECSKAIREFLSQHPNITLVIYVARLFHAMDQHNRQGLRDLINSGVTIQIMRAEEYDHCWRNFVNYSPGEEAHWPRYPPLWMTMYALELHCIILSLPPCLKITRRCQHQHTFFSLNFQNCHYQMIPPQTLYAAGIIQPSMTWR

SEQ ID NO: 7 - protein, 222 aa, Meriones unguiculatus
MSSETGPAADPTLRRRIEPQEFGAFFDPQLLRKETCLLYEINWGGRHSVWRHTGQNTDRHAEINFIEKFTSERYFCPFTRCSITWFLSWSPCGECCRAIVEFLSRYPNVTLFIYVARLYHHTDERNRQGLRDLCRRGVTIRIMTEQECYYCWRNFVNYSPSNEAHWPRYPHLWVRMYVLELYCILLGLPPCLKILRRNQNQLTIFNLAFQHCHFQRLPYYIF

SEQ ID NO: 8 - protein, 222 aa, Meriones unguiculatus
MSSETGPAADPTLRRRIEPQEFGAFFDPQLLAKETCLLYEINWGGRHSVWRHTGQNTDRHAEINFIEKFTSERYFCPFTRCSITWFLSWSPCGECCRAIVEFLSRYPNVTLFIYVARLYHHTDERNRQGLRDLCRRGVTIRIMTEQECYYCWRNFVNYSPSNEAHWPRYPHLWVRMYVLELYCILLGLPPCLKILRRNQNQLTIFNLAFQHCHFQRLPYYIF

SEQ ID NO: 9 - protein, 222 aa, Meriones unguiculatus
MSSETGPAADPTLRRRIEPQEFGAFFDPQLLRAETCLLYEINWGGRHSVWRHTGQNTDRHAEINFIEKFTSERYFCPFTRCSITWFLSWSPCGECCRAIVEFLSRYPNVTLFIYVARLYHHTDERNRQGLRDLCRRGVTIRIMTEQECYYCWRNFVNYSPSNEAHWPRYPHLWVRMYVLELYCILLGLPPCLKILRRNQNQLTIFNLAFQHCHFQRLPYYIF

SEQ ID NO: 10 - protein, 222 aa, Meriones unguiculatus
MSSETGPAADPTLRRRIEPQEFGAFFDPQLLRKETCLLYEINWGGRHSVWRHTGQNTDRHAEINFIEKFTSERYFCPFTRCSITWFLSYSPCGECCRAIVEFLSRYPNVTLFIYVARLYHHTDERNRQGLRDLCRRGVTIRIMTEQECYYCWRNFVNYSPSNEAHWPRYPHLWVRMYVLELYCILLGLPPCLKILRRNQNQLTIFNLAFQHCHFQRLPYYIF

SEQ ID NO: 11 - protein, 222 aa, Meriones unguiculatus
MSSETGPAADPTLRRRIEPQEFGAFFDPQLLRKETCLLYEINWGGRHSVWRHTGQNTDRHAEINFIEKFTSERYFCPFTRCSITWFLSYSPCGECCRAIVEFLSRYPNVTLFIYVARLYHHTDEENRQGLRDLCRRGVTIRIMTEQECYYCWRNFVNYSPSNEAHWPRYPHLWVRMYVLELYCILLGLPPCLKILRRNQNQLTIFNLAFQHCHFQRLPYYIF

SEQ ID NO: 12 - protein, 222 aa, Meriones unguiculatus
MSSETGPAADPTLRRRIEPQEFGAFFDPQLLRKETCLLYEINWGGRHSVWRHTGQNTDRHAEINFIEKFTSERYFCPFTRCSITWFLSWSPCGECCRAIVEFLSRYPNVTLFIYVARLYHATDERNRQGLRDLCRRGVTIRIMTEQECYYCWRNFVNYSPSNEAHWPRYPHLWVRMYVLELYCILLGLPPCLKILRRNQNQLTIFNLAFQHCHFQRLPYYIF

SEQ ID NO: 13 - protein, 229 aa, Myotis lucifugus
MASDAGSSAGDPTLRRRIEPWDFEAIFDPRELRKEACLLYEIKWGPCHKIWRHSGKNTTRHVEVNFIEKITSERQFCSSTSCSIIWFLSWSPCWECSKAITEFLRQRPGVTLVIYVARLYHHMDEQNRQGLRDLIKSGVTIQIMTTPEYDYCWRNFVNYPPGKDTHCPMYPPLWMKLYALELHCIILSLPPCLMISRRCQKQLTWYRLNLQNCHYQQIPPHILLATAWI

SEQ ID NO: 14 - protein, 229 aa, Myotis lucifugus
MASDAGSSAGDPTLRRRIEPWDFEAIFDPRELAKEACLLYEIKWGPCHKIWRHSGKNTTRHVEVNFIEKITSERQFCSSTSCSIIWFLSWSPCWECSKAITEFLRQRPGVTLVIYVARLYHHMDEQNRQGLRDLIKSGVTIQIMTTPEYDYCWRNFVNYPPGKDTHCPMYPPLWMKLYALELHCIILSLPPCLMISRRCQKQLTWYRLNLQNCHYQQIPPHILLATAWI

SEQ ID NO: 15 - protein, 229 aa, Myotis lucifugus
MASDAGSSAGDPTLRRRIEPWDFEAIFDPRELRAEACLLYEIKWGPCHKIWRHSGKNTTRHVEVNFIEKITSERQFCSSTSCSIIWFLSWSPCWECSKAITEFLRQRPGVTLVIYVARLYHHMDEQNRQGLRDLIKSGVTIQIMTTPEYDYCWRNFVNYPPGKDTHCPMYPPLWMKLYALELHCIILSLPPCLMISRRCQKQLTWYRLNLQNCHYQQIPPHILLATAWI

SEQ ID NO: 16 - protein, 229 aa, Myotis lucifugus
MASDAGSSAGDPTLRRRIEPWDFEAIFDPRELRKEACLLYEIKWGPCHKIWRHSGKNTTRHVEVNFIEKITSERQFCSSTSCSIIWFLSYSPCWECSKAITEFLRQRPGVTLVIYVARLYHHMDEQNRQGLRDLIKSGVTIQIMTTPEYDYCWRNFVNYPPGKDTHCPMYPPLWMKLYALELHCIILSLPPCLMISRRCQKQLTWYRLNLQNCHYQQIPPHILLATAWI

SEQ ID NO: 17 - protein, 229 aa, Myotis lucifugus
MASDAGSSAGDPTLRRRIEPWDFEAIFDPRELRKEACLLYEIKWGPCHKIWRHSGKNTTRHVEVNFIEKITSERQFCSSTSCSIIWFLSYSPCWECSKAITEFLRQRPGVTLVIYVARLYHHMDEENRQGLRDLIKSGVTIQIMTTPEYDYCWRNFVNYPPGKDTHCPMYPPLWMKLYALELHCIILSLPPCLMISRRCQKQLTWYRLNLQNCHYQQIPPHILLATAWI

SEQ ID NO: 18 - protein, 229 aa, Myotis lucifugus
MASDAGSSAGDPTLRRRIEPWDFEAIFDPRELRKEACLLYEIKWGPCHKIWRHSGKNTTRHVEVNFIEKITSERQFCSSTSCSIIWFLSWSPCWECSKAITEFLRQRPGVTLVIYVARLYHAMDEQNRQGLRDLIKSGVTIQIMTTPEYDYCWRNFVNYPPGKDTHCPMYPPLWMKLYALELHCIILSLPPCLMISRRCQKQLTWYRLNLQNCHYQQIPPHILLATAWI

SEQ ID NO: 19 - DNA, 705 bp, Dasypus novemcinctus
  source 1..705: mol_type = genomic DNA
acaagcgaaaccggccctagcaccggcgacgccaccctgcgcagacggattcagccctgggagttcgaggtgttcttcgaccctagagagctgagaaaggaaacctgcctgctgtacgagatcaagtggggcagaagcagaaagatctggagaaacagcgggcagaacacaagcaagcacgtggaggtgaacttcatcgagaagctgatcagcgagagacacttctgccctagcgtgtcttgcagcatcacctggttcctgagctggagcccctgctgggagtgcagcaaggccatcagagagttcctgtctcagcaccccaacatcaccctggtgatctacgtggctagactgttccaccacatggatcagcacaacagacaaggcctgcgggacctgatcaacagcggcgtgaccattcagatcatgagagccgaggagtacgaccactgctggagaaacttcgtgaactacagccccggcgaggaggcccactggcctagatacccccccctgtggatgaccatgtacgccctggagctgcactgcatcatcctgagcctgcccccctgcctgaagatcacaagaagatgtcagcatcagcacaccttcttcagcctgaactttcagaactgccactatcagatgatcccccctcagaccctgtacgccgccggcatcattcagcctagcatgacctggaga

SEQ ID NO: 20 - DNA, 705 bp, Dasypus novemcinctus
  source 1..705: mol_type = genomic DNA
acaagcgaaaccggccctagcaccggcgacgccaccctgcgcagacggattcagccctgggagttcgaggtgttcttcgaccctagagagctggccaaggaaacctgcctgctgtacgagatcaagtggggcagaagcagaaagatctggagaaacagcgggcagaacacaagcaagcacgtggaggtgaacttcatcgagaagctgatcagcgagagacacttctgccctagcgtgtcttgcagcatcacctggttcctgagctggagcccctgctgggagtgcagcaaggccatcagagagttcctgtctcagcaccccaacatcaccctggtgatctacgtggctagactgttccaccacatggatcagcacaacagacaaggcctgcgggacctgatcaacagcggcgtgaccattcagatcatgagagccgaggagtacgaccactgctggagaaacttcgtgaactacagccccggcgaggaggcccactggcctagatacccccccctgtggatgaccatgtacgccctggagctgcactgcatcatcctgagcctgcccccctgcctgaagatcacaagaagatgtcagcatcagcacaccttcttcagcctgaactttcagaactgccactatcagatgatcccccctcagaccctgtacgccgccggcatcattcagcctagcatgacctggaga

SEQ ID NO: 21 - DNA, 705 bp, Dasypus novemcinctus
  source 1..705: mol_type = genomic DNA
acaagcgaaaccggccctagcaccggcgacgccaccctgcgcagacggattcagccctgggagttcgaggtgttcttcgaccctagagagctgagagccgaaacctgcctgctgtacgagatcaagtggggcagaagcagaaagatctggagaaacagcgggcagaacacaagcaagcacgtggaggtgaacttcatcgagaagctgatcagcgagagacacttctgccctagcgtgtcttgcagcatcacctggttcctgagctggagcccctgctgggagtgcagcaaggccatcagagagttcctgtctcagcaccccaacatcaccctggtgatctacgtggctagactgttccaccacatggatcagcacaacagacaaggcctgcgggacctgatcaacagcggcgtgaccattcagatcatgagagccgaggagtacgaccactgctggagaaacttcgtgaactacagccccggcgaggaggcccactggcctagatacccccccctgtggatgaccatgtacgccctggagctgcactgcatcatcctgagcctgcccccctgcctgaagatcacaagaagatgtcagcatcagcacaccttcttcagcctgaactttcagaactgccactatcagatgatcccccctcagaccctgtacgccgccggcatcattcagcctagcatgacctggaga

SEQ ID NO: 22 - DNA, 705 bp, Dasypus novemcinctus
  source 1..705: mol_type = genomic DNA
acaagcgaaaccggccctagcaccggcgacgccaccctgcgcagacggattcagccctgggagttcgaggtgttcttcgaccctagagagctgagaaaggaaacctgcctgctgtacgagatcaagtggggcagaagcagaaagatctggagaaacagcgggcagaacacaagcaagcacgtggaggtgaacttcatcgagaagctgatcagcgagagacacttctgccctagcgtgtcttgcagcatcacctggttcctgagctacagcccctgctgggagtgcagcaaggccatcagagagttcctgtctcagcaccccaacatcaccctggtgatctacgtggctagactgttccaccacatggatcagcacaacagacaaggcctgcgggacctgatcaacagcggcgtgaccattcagatcatgagagccgaggagtacgaccactgctggagaaacttcgtgaactacagccccggcgaggaggcccactggcctagatacccccccctgtggatgaccatgtacgccctggagctgcactgcatcatcctgagcctgcccccctgcctgaagatcacaagaagatgtcagcatcagcacaccttcttcagcctgaactttcagaactgccactatcagatgatcccccctcagaccctgtacgccgccggcatcattcagcctagcatgacctggaga

SEQ ID NO: 23 - DNA, 705 bp, Dasypus novemcinctus
  source 1..705: mol_type = genomic DNA
acaagcgaaaccggccctagcaccggcgacgccaccctgcgcagacggattcagccctgggagttcgaggtgttcttcgaccctagagagctgagaaaggaaacctgcctgctgtacgagatcaagtggggcagaagcagaaagatctggagaaacagcgggcagaacacaagcaagcacgtggaggtgaacttcatcgagaagctgatcagcgagagacacttctgccctagcgtgtcttgcagcatcacctggttcctgagctacagcccctgctgggagtgcagcaaggccatcagagagttcctgtctcagcaccccaacatcaccctggtgatctacgtggctagactgttccaccacatggatcaggagaacagacaaggcctgcgggacctgatcaacagcggcgtgaccattcagatcatgagagccgaggagtacgaccactgctggagaaacttcgtgaactacagccccggcgaggaggcccactggcctagatacccccccctgtggatgaccatgtacgccctggagctgcactgcatcatcctgagcctgcccccctgcctgaagatcacaagaagatgtcagcatcagcacaccttcttcagcctgaactttcagaactgccactatcagatgatcccccctcagaccctgtacgccgccggcatcattcagcctagcatgacctggaga

SEQ ID NO: 24 - DNA, 705 bp, Dasypus novemcinctus
  source 1..705: mol_type = genomic DNA
acaagcgaaaccggccctagcaccggcgacgccaccctgcgcagacggattcagccctgggagttcgaggtgttcttcgaccctagagagctgagaaaggaaacctgcctgctgtacgagatcaagtggggcagaagcagaaagatctggagaaacagcgggcagaacacaagcaagcacgtggaggtgaacttcatcgagaagctgatcagcgagagacacttctgccctagcgtgtcttgcagcatcacctggttcctgagctggagcccctgctgggagtgcagcaaggccatcagagagttcctgtctcagcaccccaacatcaccctggtgatctacgtggctagactgttccacgccatggatcagcacaacagacaaggcctgcgggacctgatcaacagcggcgtgaccattcagatcatgagagccgaggagtacgaccactgctggagaaacttcgtgaactacagccccggcgaggaggcccactggcctagatacccccccctgtggatgaccatgtacgccctggagctgcactgcatcatcctgagcctgcccccctgcctgaagatcacaagaagatgtcagcatcagcacaccttcttcagcctgaactttcagaactgccactatcagatgatcccccctcagaccctgtacgccgccggcatcattcagcctagcatgacctggaga

SEQ ID NO: 25 - DNA, 663 bp, Meriones unguiculatus
  source 1..663: mol_type = genomic DNA
agcagcgaaaccggccccgccgccgaccccaccctgagaagaagaatcgagccccaagagttcggcgccttcttcgaccctcagctgctgagaaaggaaacctgcctgctgtacgagatcaactggggcggcagacacagcgtgtggagacacaccgggcagaacaccgacagacacgccgagatcaacttcatcgagaagttcacaagcgagagatacttctgccccttcacaagatgcagcatcacctggttcctgagctggagcccctgcggcgagtgttgcagagccatcgtggagttcctgagcagataccccaacgtgaccctgttcatctacgtggctagactgtaccaccacaccgacgagagaaacagacaaggcctgcgggacctgtgcagaagaggcgtgaccatcagaatcatgaccgagcaagagtgctactactgctggagaaacttcgtgaactacagccctagcaacgaggcccactggcctagatacccccacctgtgggtgagaatgtacgtgctggagctgtactgcatcctgctgggcctgcccccctgcctgaagatcctgagaagaaatcagaatcagctgaccatcttcaacctggcctttcagcactgccactttcagagactgccctactacatcttc

SEQ ID NO: 26 - DNA, 663 bp, Meriones unguiculatus
  source 1..663: mol_type = genomic DNA
agcagcgaaaccggccccgccgccgaccccaccctgagaagaagaatcgagccccaagagttcggcgccttcttcgaccctcagctgctggccaaggaaacctgcctgctgtacgagatcaactggggcggcagacacagcgtgtggagacacaccgggcagaacaccgacagacacgccgagatcaacttcatcgagaagttcacaagcgagagatacttctgccccttcacaagatgcagcatcacctggttcctgagctggagcccctgcggcgagtgttgcagagccatcgtggagttcctgagcagataccccaacgtgaccctgttcatctacgtggctagactgtaccaccacaccgacgagagaaacagacaaggcctgcgggacctgtgcagaagaggcgtgaccatcagaatcatgaccgagcaagagtgctactactgctggagaaacttcgtgaactacagccctagcaacgaggcccactggcctagatacccccacctgtgggtgagaatgtacgtgctggagctgtactgcatcctgctgggcctgcccccctgcctgaagatcctgagaagaaatcagaatcagctgaccatcttcaacctggcctttcagcactgccactttcagagactgccctactacatcttc

SEQ ID NO: 27 - DNA, 663 bp, Meriones unguiculatus
  source 1..663: mol_type = genomic DNA
agcagcgaaaccggccccgccgccgaccccaccctgagaagaagaatcgagccccaagagttcggcgccttcttcgaccctcagctgctgagagccgaaacctgcctgctgtacgagatcaactggggcggcagacacagcgtgtggagacacaccgggcagaacaccgacagacacgccgagatcaacttcatcgagaagttcacaagcgagagatacttctgccccttcacaagatgcagcatcacctggttcctgagctggagcccctgcggcgagtgttgcagagccatcgtggagttcctgagcagataccccaacgtgaccctgttcatctacgtggctagactgtaccaccacaccgacgagagaaacagacaaggcctgcgggacctgtgcagaagaggcgtgaccatcagaatcatgaccgagcaagagtgctactactgctggagaaacttcgtgaactacagccctagcaacgaggcccactggcctagatacccccacctgtgggtgagaatgtacgtgctggagctgtactgcatcctgctgggcctgcccccctgcctgaagatcctgagaagaaatcagaatcagctgaccatcttcaacctggcctttcagcactgccactttcagagactgccctactacatcttc

SEQ ID NO: 28 - DNA, 663 bp, Meriones unguiculatus
  source 1..663: mol_type = genomic DNA
agcagcgaaaccggccccgccgccgaccccaccctgagaagaagaatcgagccccaagagttcggcgccttcttcgaccctcagctgctgagaaaggaaacctgcctgctgtacgagatcaactggggcggcagacacagcgtgtggagacacaccgggcagaacaccgacagacacgccgagatcaacttcatcgagaagttcacaagcgagagatacttctgccccttcacaagatgcagcatcacctggttcctgagctacagcccctgcggcgagtgttgcagagccatcgtggagttcctgagcagataccccaacgtgaccctgttcatctacgtggctagactgtaccaccacaccgacgagagaaacagacaaggcctgcgggacctgtgcagaagaggcgtgaccatcagaatcatgaccgagcaagagtgctactactgctggagaaacttcgtgaactacagccctagcaacgaggcccactggcctagatacccccacctgtgggtgagaatgtacgtgctggagctgtactgcatcctgctgggcctgcccccctgcctgaagatcctgagaagaaatcagaatcagctgaccatcttcaacctggcctttcagcactgccactttcagagactgccctactacatcttc

SEQ ID NO: 29 - DNA, 663 bp, Meriones unguiculatus
  source 1..663: mol_type = genomic DNA
agcagcgaaaccggccccgccgccgaccccaccctgagaagaagaatcgagccccaagagttcggcgccttcttcgaccctcagctgctgagaaaggaaacctgcctgctgtacgagatcaactggggcggcagacacagcgtgtggagacacaccgggcagaacaccgacagacacgccgagatcaacttcatcgagaagttcacaagcgagagatacttctgccccttcacaagatgcagcatcacctggttcctgagctacagcccctgcggcgagtgttgcagagccatcgtggagttcctgagcagataccccaacgtgaccctgttcatctacgtggctagactgtaccaccacaccgacgaggagaacagacaaggcctgcgggacctgtgcagaagaggcgtgaccatcagaatcatgaccgagcaagagtgctactactgctggagaaacttcgtgaactacagccctagcaacgaggcccactggcctagatacccccacctgtgggtgagaatgtacgtgctggagctgtactgcatcctgctgggcctgcccccctgcctgaagatcctgagaagaaatcagaatcagctgaccatcttcaacctggcctttcagcactgccactttcagagactgccctactacatcttc

SEQ ID NO: 30 - DNA, 663 bp, Meriones unguiculatus
  source 1..663: mol_type = genomic DNA
agcagcgaaaccggccccgccgccgaccccaccctgagaagaagaatcgagccccaagagttcggcgccttcttcgaccctcagctgctgagaaaggaaacctgcctgctgtacgagatcaactggggcggcagacacagcgtgtggagacacaccgggcagaacaccgacagacacgccgagatcaacttcatcgagaagttcacaagcgagagatacttctgccccttcacaagatgcagcatcacctggttcctgagctggagcccctgcggcgagtgttgcagagccatcgtggagttcctgagcagataccccaacgtgaccctgttcatctacgtggctagactgtaccacgccaccgacgagagaaacagacaaggcctgcgggacctgtgcagaagaggcgtgaccatcagaatcatgaccgagcaagagtgctactactgctggagaaacttcgtgaactacagccctagcaacgaggcccactggcctagatacccccacctgtgggtgagaatgtacgtgctggagctgtactgcatcctgctgggcctgcccccctgcctgaagatcctgagaagaaatcagaatcagctgaccatcttcaacctggcctttcagcactgccactttcagagactgccctactacatcttc

SEQ ID NO: 31 - DNA, 684 bp, Myotis lucifugus
  source 1..684: mol_type = genomic DNA
gcaagcgacgccggcagcagcgccggcgaccccaccctgagaagaagaatcgagccctgggacttcgaggccatcttcgaccctagagagctgagaaaggaggcctgcctgctgtacgagatcaagtggggcccctgccacaagatctggagacacagcggcaagaacaccacaagacacgtggaggtgaacttcatcgagaagatcacaagcgagagacagttttgcagcagcacatcttgcagcatcatctggttcctgagctggagcccctgctgggagtgcagcaaggccatcaccgagttcctgagacaaagacccggcgtgaccctggtgatctacgtggctagactgtaccaccacatggacgagcagaacagacaaggcctgcgggacctgatcaagagcggcgtgaccattcagatcatgaccacccccgagtacgactactgctggagaaacttcgtgaactacccccccggcaaggacacccactgccccatgtacccccccctgtggatgaagctgtacgccctggagctgcactgcatcatcctgagcctgcccccctgcctgatgatcagcagaagatgtcagaagcagctgacctggtacagactgaacttgcagaactgccactatcagcagatccccccccacatcctgctggccaccgcctggatc

SEQ ID NO: 32 - DNA, 684 bp, Myotis lucifugus
  source 1..684: mol_type = genomic DNA
gcaagcgacgccggcagcagcgccggcgaccccaccctgagaagaagaatcgagccctgggacttcgaggccatcttcgaccctagagagctggccaaggaggcctgcctgctgtacgagatcaagtggggcccctgccacaagatctggagacacagcggcaagaacaccacaagacacgtggaggtgaacttcatcgagaagatcacaagcgagagacagttttgcagcagcacatcttgcagcatcatctggttcctgagctggagcccctgctgggagtgcagcaaggccatcaccgagttcctgagacaaagacccggcgtgaccctggtgatctacgtggctagactgtaccaccacatggacgagcagaacagacaaggcctgcgggacctgatcaagagcggcgtgaccattcagatcatgaccacccccgagtacgactactgctggagaaacttcgtgaactacccccccggcaaggacacccactgccccatgtacccccccctgtggatgaagctgtacgccctggagctgcactgcatcatcctgagcctgcccccctgcctgatgatcagcagaagatgtcagaagcagctgacctggtacagactgaacttgcagaactgccactatcagcagatccccccccacatcctgctggccaccgcctggatc

SEQ ID NO: 33 - DNA, 684 bp, Myotis lucifugus
  source 1..684: mol_type = genomic DNA
gcaagcgacgccggcagcagcgccggcgaccccaccctgagaagaagaatcgagccctgggacttcgaggccatcttcgaccctagagagctgagagccgaggcctgcctgctgtacgagatcaagtggggcccctgccacaagatctggagacacagcggcaagaacaccacaagacacgtggaggtgaacttcatcgagaagatcacaagcgagagacagttttgcagcagcacatcttgcagcatcatctggttcctgagctggagcccctgctgggagtgcagcaaggccatcaccgagttcctgagacaaagacccggcgtgaccctggtgatctacgtggctagactgtaccaccacatggacgagcagaacagacaaggcctgcgggacctgatcaagagcggcgtgaccattcagatcatgaccacccccgagtacgactactgctggagaaacttcgtgaactacccccccggcaaggacacccactgccccatgtacccccccctgtggatgaagctgtacgccctggagctgcactgcatcatcctgagcctgcccccctgcctgatgatcagcagaagatgtcagaagcagctgacctggtacagactgaacttgcagaactgccactatcagcagatccccccccacatcctgctggccaccgcctggatc

SEQ ID NO: 34 - DNA, 684 bp, Myotis lucifugus
  source 1..684: mol_type = genomic DNA
gcaagcgacgccggcagcagcgccggcgaccccaccctgagaagaagaatcgagccctgggacttcgaggccatcttcgaccctagagagctgagaaaggaggcctgcctgctgtacgagatcaagtggggcccctgccacaagatctggagacacagcggcaagaacaccacaagacacgtggaggtgaacttcatcgagaagatcacaagcgagagacagttttgcagcagcacatcttgcagcatcatctggttcctgagctacagcccctgctgggagtgcagcaaggccatcaccgagttcctgagacaaagacccggcgtgaccctggtgatctacgtggctagactgtaccaccacatggacgagcagaacagacaaggcctgcgggacctgatcaagagcggcgtgaccattcagatcatgaccacccccgagtacgactactgctggagaaacttcgtgaactacccccccggcaaggacacccactgccccatgtacccccccctgtggatgaagctgtacgccctggagctgcactgcatcatcctgagcctgcccccctgcctgatgatcagcagaagatgtcagaagcagctgacctggtacagactgaacttgcagaactgccactatcagcagatccccccccacatcctgctggccaccgcctggatc

SEQ ID NO: 35 - DNA, 684 bp, Myotis lucifugus
  source 1..684: mol_type = genomic DNA
gcaagcgacgccggcagcagcgccggcgaccccaccctgagaagaagaatcgagccctgggacttcgaggccatcttcgaccctagagagctgagaaaggaggcctgcctgctgtacgagatcaagtggggcccctgccacaagatctggagacacagcggcaagaacaccacaagacacgtggaggtgaacttcatcgagaagatcacaagcgagagacagttttgcagcagcacatcttgcagcatcatctggttcctgagctacagcccctgctgggagtgcagcaaggccatcaccgagttcctgagacaaagacccggcgtgaccctggtgatctacgtggctagactgtaccaccacatggacgaggagaacagacaaggcctgcgggacctgatcaagagcggcgtgaccattcagatcatgaccacccccgagtacgactactgctggagaaacttcgtgaactacccccccggcaaggacacccactgccccatgtacccccccctgtggatgaagctgtacgccctggagctgcactgcatcatcctgagcctgcccccctgcctgatgatcagcagaagatgtcagaagcagctgacctggtacagactgaacttgcagaactgccactatcagcagatccccccccacatcctgctggccaccgcctggatc

SEQ ID NO: 36 - DNA, 684 bp, Myotis lucifugus
  source 1..684: mol_type = genomic DNA
gcaagcgacgccggcagcagcgccggcgaccccaccctgagaagaagaatcgagccctgggacttcgaggccatcttcgaccctagagagctgagaaaggaggcctgcctgctgtacgagatcaagtggggcccctgccacaagatctggagacacagcggcaagaacaccacaagacacgtggaggtgaacttcatcgagaagatcacaagcgagagacagttttgcagcagcacatcttgcagcatcatctggttcctgagctggagcccctgctgggagtgcagcaaggccatcaccgagttcctgagacaaagacccggcgtgaccctggtgatctacgtggctagactgtaccacgccatggacgagcagaacagacaaggcctgcgggacctgatcaagagcggcgtgaccattcagatcatgaccacccccgagtacgactactgctggagaaacttcgtgaactacccccccggcaaggacacccactgccccatgtacccccccctgtggatgaagctgtacgccctggagctgcactgcatcatcctgagcctgcccccctgcctgatgatcagcagaagatgtcagaagcagctgacctggtacagactgaacttgcagaactgccactatcagcagatccccccccacatcctgctggccaccgcctggatc

SEQ ID NO: 37 - DNA, 745 bp, synthetic construct
  misc_feature 1..745: note = Synthetic polynucleotide
  source 1..745: mol_type = other DNA
atcgctacggtctcggctccacaagcgaaaccggccctagcaccggcgacgccaccctgcgcagacggattcagccctgggagttcgaggtgttcttcgaccctagagagctgagaaaggaaacctgcctgctgtacgagatcaagtggggcagaagcagaaagatctggagaaacagcgggcagaacacaagcaagcacgtggaggtgaacttcatcgagaagctgatcagcgagagacacttctgccctagcgtgtcttgcagcatcacctggttcctgagctggagcccctgctgggagtgcagcaaggccatcagagagttcctgtctcagcaccccaacatcaccctggtgatctacgtggctagactgttccaccacatggatcagcacaacagacaaggcctgcgggacctgatcaacagcggcgtgaccattcagatcatgagagccgaggagtacgaccactgctggagaaacttcgtgaactacagccccggcgaggaggcccactggcctagatacccccccctgtggatgaccatgtacgccctggagctgcactgcatcatcctgagcctgcccccctgcctgaagatcacaagaagatgtcagcatcagcacaccttcttcagcctgaactttcagaactgccactatcagatgatcccccctcagaccctgtacgccgccggcatcattcagcctagcatgacctggagatctggagagaccgtagcgat

SEQ ID NO: 38 - DNA, 745 bp, synthetic construct
  misc_feature 1..745: note = Synthetic polynucleotide
  source 1..745: mol_type = other DNA
atcgctacggtctcggctccacaagcgaaaccggccctagcaccggcgacgccaccctgcgcagacggattcagccctgggagttcgaggtgttcttcgaccctagagagctggccaaggaaacctgcctgctgtacgagatcaagtggggcagaagcagaaagatctggagaaacagcgggcagaacacaagcaagcacgtggaggtgaacttcatcgagaagctgatcagcgagagacacttctgccctagcgtgtcttgcagcatcacctggttcctgagctggagcccctgctgggagtgcagcaaggccatcagagagttcctgtctcagcaccccaacatcaccctggtgatctacgtggctagactgttccaccacatggatcagcacaacagacaaggcctgcgggacctgatcaacagcggcgtgaccattcagatcatgagagccgaggagtacgaccactgctggagaaacttcgtgaactacagccccggcgaggaggcccactggcctagatacccccccctgtggatgaccatgtacgccctggagctgcactgcatcatcctgagcctgcccccctgcctgaagatcacaagaagatgtcagcatcagcacaccttcttcagcctgaactttcagaactgccactatcagatgatcccccctcagaccctgtacgccgccggcatcattcagcctagcatgacctggagatctggagagaccgtagcgat

SEQ ID NO: 39 - DNA, 745 bp, synthetic construct
  misc_feature 1..745: note = Synthetic polynucleotide
  source 1..745: mol_type = other DNA
atcgctacggtctcggctccacaagcgaaaccggccctagcaccggcgacgccaccctgcgcagacggattcagccctgggagttcgaggtgttcttcgaccctagagagctgagagccgaaacctgcctgctgtacgagatcaagtggggcagaagcagaaagatctggagaaacagcgggcagaacacaagcaagcacgtggaggtgaacttcatcgagaagctgatcagcgagagacacttctgccctagcgtgtcttgcagcatcacctggttcctgagctggagcccctgctgggagtgcagcaaggccatcagagagttcctgtctcagcaccccaacatcaccctggtgatctacgtggctagactgttccaccacatggatcagcacaacagacaaggcctgcgggacctgatcaacagcggcgtgaccattcagatcatgagagccgaggagtacgaccactgctggagaaacttcgtgaactacagccccggcgaggaggcccactggcctagatacccccccctgtggatgaccatgtacgccctggagctgcactgcatcatcctgagcctgcccccctgcctgaagatcacaagaagatgtcagcatcagcacaccttcttcagcctgaactttcagaactgccactatcagatgatcccccctcagaccctgtacgccgccggcatcattcagcctagcatgacctggagatctggagagaccgtagcgat

SEQ ID NO: 40 - DNA, 745 bp, synthetic construct
  misc_feature 1..745: note = Synthetic polynucleotide
  source 1..745: mol_type = other DNA
atcgctacggtctcggctccacaagcgaaaccggccctagcaccggcgacgccaccctgcgcagacggattcagccctgggagttcgaggtgttcttcgaccctagagagctgagaaaggaaacctgcctgctgtacgagatcaagtggggcagaagcagaaagatctggagaaacagcgggcagaacacaagcaagcacgtggaggtgaacttcatcgagaagctgatcagcgagagacacttctgccctagcgtgtcttgcagcatcacctggttcctgagctacagcccctgctgggagtgcagcaaggccatcagagagttcctgtctcagcaccccaacatcaccctggtgatctacgtggctagactgttccaccacatggatcagcacaacagacaaggcctgcgggacctgatcaacagcggcgtgaccattcagatcatgagagccgaggagtacgaccactgctggagaaacttcgtgaactacagccccggcgaggaggcccactggcctagatacccccccctgtggatgaccatgtacgccctggagctgcactgcatcatcctgagcctgcccccctgcctgaagatcacaagaagatgtcagcatcagcacaccttcttcagcctgaactttcagaactgccactatcagatgatcccccctcagaccctgtacgccgccggcatcattcagcctagcatgacctggagatctggagagaccgtagcgat

SEQ ID NO: 41 - DNA, 745 bp, synthetic construct
  misc_feature 1..745: note = Synthetic polynucleotide
  source 1..745: mol_type = other DNA
atcgctacggtctcggctccacaagcgaaaccggccctagcaccggcgacgccaccctgcgcagacggattcagccctgggagttcgaggtgttcttcgaccctagagagctgagaaaggaaacctgcctgctgtacgagatcaagtggggcagaagcagaaagatctggagaaacagcgggcagaacacaagcaagcacgtggaggtgaacttcatcgagaagctgatcagcgagagacacttctgccctagcgtgtcttgcagcatcacctggttcctgagctacagcccctgctgggagtgcagcaaggccatcagagagttcctgtctcagcaccccaacatcaccctggtgatctacgtggctagactgttccaccacatggatcaggagaacagacaaggcctgcgggacctgatcaacagcggcgtgaccattcagatcatgagagccgaggagtacgaccactgctggagaaacttcgtgaactacagccccggcgaggaggcccactggcctagatacccccccctgtggatgaccatgtacgccctggagctgcactgcatcatcctgagcctgcccccctgcctgaagatcacaagaagatgtcagcatcagcacaccttcttcagcctgaactttcagaactgccactatcagatgatcccccctcagaccctgtacgccgccggcatcattcagcctagcatgacctggagatctggagagaccgtagcgat

SEQ ID NO: 42 - DNA, 745 bp, synthetic construct
  misc_feature 1..745: note = Synthetic polynucleotide
  source 1..745: mol_type = other DNA
atcgctacggtctcggctccacaagcgaaaccggccctagcaccggcgacgccaccctgcgcagacggattcagccctgggagttcgaggtgttcttcgaccctagagagctgagaaaggaaacctgcctgctgtacgagatcaagtggggcagaagcagaaagatctggagaaacagcgggcagaacacaagcaagcacgtggaggtgaacttcatcgagaagctgatcagcgagagacacttctgccctagcgtgtcttgcagcatcacctggttcctgagctggagcccctgctgggagtgcagcaaggccatcagagagttcctgtctcagcaccccaacatcaccctggtgatctacgtggctagactgttccacgccatggatcagcacaacagacaaggcctgcgggacctgatcaacagcggcgtgaccattcagatcatgagagccgaggagtacgaccactgctggagaaacttcgtgaactacagccccggcgaggaggcccactggcctagatacccccccctgtggatgaccatgtacgccctggagctgcactgcatcatcctgagcctgcccccctgcctgaagatcacaagaagatgtcagcatcagcacaccttcttcagcctgaactttcagaactgccactatcagatgatcccccctcagaccctgtacgccgccggcatcattcagcctagcatgacctggagatctggagagaccgtagcgat

SEQ ID NO: 43 - DNA, 703 bp, synthetic construct
  misc_feature 1..703: note = Synthetic polynucleotide
  source 1..703: mol_type = other DNA
atcgctacggtctcggctccagcagcgaaaccggccccgccgccgaccccaccctgagaagaagaatcgagccccaagagttcggcgccttcttcgaccctcagctgctgagaaaggaaacctgcctgctgtacgagatcaactggggcggcagacacagcgtgtggagacacaccgggcagaacaccgacagacacgccgagatcaacttcatcgagaagttcacaagcgagagatacttctgccccttcacaagatgcagcatcacctggttcctgagctggagcccctgcggcgagtgttgcagagccatcgtggagttcctgagcagataccccaacgtgaccctgttcatctacgtggctagactgtaccaccacaccgacgagagaaacagacaaggcctgcgggacctgtgcagaagaggcgtgaccatcagaatcatgaccgagcaagagtgctactactgctggagaaacttcgtgaactacagccctagcaacgaggcccactggcctagatacccccacctgtgggtgagaatgtacgtgctggagctgtactgcatcctgctgggcctgcccccctgcctgaagatcctgagaagaaatcagaatcagctgaccatcttcaacctggcctttcagcactgccactttcagagactgccctactacatcttctctggagagaccgtagcgat

SEQ ID NO: 44 - DNA, 703 bp, synthetic construct
  misc_feature 1..703: note = Synthetic polynucleotide
  source 1..703: mol_type = other DNA
atcgctacggtctcggctccagcagcgaaaccggccccgccgccgaccccaccctgagaagaagaatcgagccccaagagttcggcgccttcttcgaccctcagctgctggccaaggaaacctgcctgctgtacgagatcaactggggcggcagacacagcgtgtggagacacaccgggcagaacaccgacagacacgccgagatcaacttcatcgagaagttcacaagcgagagatacttctgccccttcacaagatgcagcatcacctggttcctgagctggagcccctgcggcgagtgttgcagagccatcgtggagttcctgagcagataccccaacgtgaccctgttcatctacgtggctagactgtaccaccacaccgacgagagaaacagacaaggcctgcgggacctgtgcagaagaggcgtgaccatcagaatcatgaccgagcaagagtgctactactgctggagaaacttcgtgaactacagccctagcaacgaggcccactggcctagatacccccacctgtgggtgagaatgtacgtgctggagctgtactgcatcctgctgggcctgcccccctgcctgaagatcctgagaagaaatcagaatcagctgaccatcttcaacctggcctttcagcactgccactttcagagactgccctactacatcttctctggagagaccgtagcgat

SEQ ID NO: 45 - DNA, 703 bp, synthetic construct
  misc_feature 1..703: note = Synthetic polynucleotide
  source 1..703: mol_type = other DNA
atcgctacggtctcggctccagcagcgaaaccggccccgccgccgaccccaccctgagaagaagaatcgagccccaagagttcggcgccttcttcgaccctcagctgctgagagccgaaacctgcctgctgtacgagatcaactggggcggcagacacagcgtgtggagacacaccgggcagaacaccgacagacacgccgagatcaacttcatcgagaagttcacaagcgagagatacttctgccccttcacaagatgcagcatcacctggttcctgagctggagcccctgcggcgagtgttgcagagccatcgtggagttcctgagcagataccccaacgtgaccctgttcatctacgtggctagactgtaccaccacaccgacgagagaaacagacaaggcctgcgggacctgtgcagaagaggcgtgaccatcagaatcatgaccgagcaagagtgctactactgctggagaaacttcgtgaactacagccctagcaacgaggcccactggcctagatacccccacctgtgggtgagaatgtacgtgctggagctgtactgcatcctgctgggcctgcccccctgcctgaagatcctgagaagaaatcagaatcagctgaccatcttcaacctggcctttcagcactgccactttcagagactgccctactacatcttctctggagagaccgtagcgat

SEQ ID NO: 46 - DNA, 703 bp, synthetic construct
  misc_feature 1..703: note = Synthetic polynucleotide
  source 1..703: mol_type = other DNA
atcgctacggtctcggctccagcagcgaaaccggccccgccgccgaccccaccctgagaagaagaatcgagccccaagagttcggcgccttcttcgaccctcagctgctgagaaaggaaacctgcctgctgtacgagatcaactggggcggcagacacagcgtgtggagacacaccgggcagaacaccgacagacacgccgagatcaacttcatcgagaagttcacaagcgagagatacttctgccccttcacaagatgcagcatcacctggttcctgagctacagcccctgcggcgagtgttgcagagccatcgtggagttcctgagcagataccccaacgtgaccctgttcatctacgtggctagactgtaccaccacaccgacgagagaaacagacaaggcctgcgggacctgtgcagaagaggcgtgaccatcagaatcatgaccgagcaagagtgctactactgctggagaaacttcgtgaactacagccctagcaacgaggcccactggcctagatacccccacctgtgggtgagaatgtacgtgctggagctgtactgcatcctgctgggcctgcccccctgcctgaagatcctgagaagaaatcagaatcagctgaccatcttcaacctggcctttcagcactgccactttcagagactgccctactacatcttctctggagagaccgtagcgat

SEQ ID NO: 47 - DNA, 703 bp, synthetic construct
  misc_feature 1..703: note = Synthetic polynucleotide
  source 1..703: mol_type = other DNA
atcgctacggtctcggctccagcagcgaaaccggccccgccgccgaccccaccctgagaagaagaatcgagccccaagagttcggcgccttcttcgaccctcagctgctgagaaaggaaacctgcctgctgtacgagatcaactggggcggcagacacagcgtgtggagacacaccgggcagaacaccgacagacacgccgagatcaacttcatcgagaagttcacaagcgagagatacttctgccccttcacaagatgcagcatcacctggttcctgagctacagcccctgcggcgagtgttgcagagccatcgtggagttcctgagcagataccccaacgtgaccctgttcatctacgtggctagactgtaccaccacaccgacgaggagaacagacaaggcctgcgggacctgtgcagaagaggcgtgaccatcagaatcatgaccgagcaagagtgctactactgctggagaaacttcgtgaactacagccctagcaacgaggcccactggcctagatacccccacctgtgggtgagaatgtacgtgctggagctgtactgcatcctgctgggcctgcccccctgcctgaagatcctgagaagaaatcagaatcagctgaccatcttcaacctggcctttcagcactgccactttcagagactgccctactacatcttctctggagagaccgtagcgat

SEQ ID NO: 48 - DNA, 703 bp, synthetic construct
  misc_feature 1..703: note = Synthetic polynucleotide
  source 1..703: mol_type = other DNA
atcgctacggtctcggctccagcagcgaaaccggccccgccgccgaccccaccctgagaagaagaatcgagccccaagagttcggcgccttcttcgaccctcagctgctgagaaaggaaacctgcctgctgtacgagatcaactggggcggcagacacagcgtgtggagacacaccgggcagaacaccgacagacacgccgagatcaacttcatcgagaagttcacaagcgagagatacttctgccccttcacaagatgcagcatcacctggttcctgagctggagcccctgcggcgagtgttgcagagccatcgtggagttcctgagcagataccccaacgtgaccctgttcatctacgtggctagactgtaccacgccaccgacgagagaaacagacaaggcctgcgggacctgtgcagaagaggcgtgaccatcagaatcatgaccgagcaagagtgctactactgctggagaaacttcgtgaactacagccctagcaacgaggcccactggcctagatacccccacctgtgggtgagaatgtacgtgctggagctgtactgcatcctgctgggcctgcccccctgcctgaagatcctgagaagaaatcagaatcagctgaccatcttcaacctggcctttcagcactgccactttcagagactgccctactacatcttctctggagagaccgtagcgat

SEQ ID NO: 49 - DNA, 724 bp, synthetic construct
  misc_feature 1..724: note = Synthetic polynucleotide
  source 1..724: mol_type = other DNA
atcgctacggtctcggctccgcaagcgacgccggcagcagcgccggcgaccccaccctgagaagaagaatcgagccctgggacttcgaggccatcttcgaccctagagagctgagaaaggaggcctgcctgctgtacgagatcaagtggggcccctgccacaagatctggagacacagcggcaagaacaccacaagacacgtggaggtgaacttcatcgagaagatcacaagcgagagacagttttgcagcagcacatcttgcagcatcatctggttcctgagctggagcccctgctgggagtgcagcaaggccatcaccgagttcctgagacaaagacccggcgtgaccctggtgatctacgtggctagactgtaccaccacatggacgagcagaacagacaaggcctgcgggacctgatcaagagcggcgtgaccattcagatcatgaccacccccgagtacgactactgctggagaaacttcgtgaactacccccccggcaaggacacccactgccccatgtacccccccctgtggatgaagctgtacgccctggagctgcactgcatcatcctgagcctgcccccctgcctgatgatcagcagaagatgtcagaagcagctgacctggtacagactgaacttgcagaactgccactatcagcagatccccccccacatcctgctggccaccgcctggatctctggagagaccgtagcgat

SEQ ID NO: 50 - DNA, 724 bp, synthetic construct
  misc_feature 1..724: note = Synthetic polynucleotide
  source 1..724: mol_type = other DNA
atcgctacggtctcggctccgcaagcgacgccggcagcagcgccggcgaccccaccctgagaagaagaatcgagccctgggacttcgaggccatcttcgaccctagagagctggccaaggaggcctgcctgctgtacgagatcaagtggggcccctgccacaagatctggagacacagcggcaagaacaccacaagacacgtggaggtgaacttcatcgagaagatcacaagcgagagacagttttgcagcagcacatcttgcagcatcatctggttcctgagctggagcccctgctgggagtgcagcaaggccatcaccgagttcctgagacaaagacccggcgtgaccctggtgatctacgtggctagactgtaccaccacatggacgagcagaacagacaaggcctgcgggacctgatcaagagcggcgtgaccattcagatcatgaccacccccgagtacgactactgctggagaaacttcgtgaactacccccccggcaaggacacccactgccccatgtacccccccctgtggatgaagctgtacgccctggagctgcactgcatcatcctgagcctgcccccctgcctgatgatcagcagaagatgtcagaagcagctgacctggtacagactgaacttgcagaactgccactatcagcagatccccccccacatcctgctggccaccgcctggatctctggagagaccgtagcgat

SEQ ID NO: 51 - DNA, 724 bp, synthetic construct
  misc_feature 1..724: note = Synthetic polynucleotide
  source 1..724: mol_type = other DNA
atcgctacggtctcggctccgcaagcgacgccggcagcagcgccggcgaccccaccctgagaagaagaatcgagccctgggacttcgaggccatcttcgaccctagagagctgagagccgaggcctgcctgctgtacgagatcaagtggggcccctgccacaagatctggagacacagcggcaagaacaccacaagacacgtggaggtgaacttcatcgagaagatcacaagcgagagacagttttgcagcagcacatcttgcagcatcatctggttcctgagctggagcccctgctgggagtgcagcaaggccatcaccgagttcctgagacaaagacccggcgtgaccctggtgatctacgtggctagactgtaccaccacatggacgagcagaacagacaaggcctgcgggacctgatcaagagcggcgtgaccattcagatcatgaccacccccgagtacgactactgctggagaaacttcgtgaactacccccccggcaaggacacccactgccccatgtacccccccctgtggatgaagctgtacgccctggagctgcactgcatcatcctgagcctgcccccctgcctgatgatcagcagaagatgtcagaagcagctgacctggtacagactgaacttgcagaactgccactatcagcagatccccccccacatcctgctggccaccgcctggatctctggagagaccgtagcgat

SEQ ID NO: 52 - DNA, 724 bp, synthetic construct
  misc_feature 1..724: note = Synthetic polynucleotide
  source 1..724: mol_type = other DNA
atcgctacggtctcggctccgcaagcgacgccggcagcagcgccggcgaccccaccctgagaagaagaatcgagccctgggacttcgaggccatcttcgaccctagagagctgagaaaggaggcctgcctgctgtacgagatcaagtggggcccctgccacaagatctggagacacagcggcaagaacaccacaagacacgtggaggtgaacttcatcgagaagatcacaagcgagagacagttttgcagcagcacatcttgcagcatcatctggttcctgagctacagcccctgctgggagtgcagcaaggccatcaccgagttcctgagacaaagacccggcgtgaccctggtgatctacgtggctagactgtaccaccacatggacgagcagaacagacaaggcctgcgggacctgatcaagagcggcgtgaccattcagatcatgaccacccccgagtacgactactgctggagaaacttcgtgaactacccccccggcaaggacacccactgccccatgtacccccccctgtggatgaagctgtacgccctggagctgcactgcatcatcctgagcctgcccccctgcctgatgatcagcagaagatgtcagaagcagctgacctggtacagactgaacttgcagaactgccactatcagcagatccccccccacatcctgctggccaccgcctggatctctggagagaccgtagcgat

SEQ ID NO: 53 - DNA, 724 bp, synthetic construct
  misc_feature 1..724: note = Synthetic polynucleotide
  source 1..724: mol_type = other DNA
atcgctacggtctcggctccgcaagcgacgccggcagcagcgccggcgaccccaccctgagaagaagaatcgagccctgggacttcgaggccatcttcgaccctagagagctgagaaaggaggcctgcctgctgtacgagatcaagtggggcccctgccacaagatctggagacacagcggcaagaacaccacaagacacgtggaggtgaacttcatcgagaagatcacaagcgagagacagttttgcagcagcacatcttgcagcatcatctggttcctgagctacagcccctgctgggagtgcagcaaggccatcaccgagttcctgagacaaagacccggcgtgaccctggtgatctacgtggctagactgtaccaccacatggacgaggagaacagacaaggcctgcgggacctgatcaagagcggcgtgaccattcagatcatgaccacccccgagtacgactactgctggagaaacttcgtgaactacccccccggcaaggacacccactgccccatgtacccccccctgtggatgaagctgtacgccctggagctgcactgcatcatcctgagcctgcccccctgcctgatgatcagcagaagatgtcagaagcagctgacctggtacagactgaacttgcagaactgccactatcagcagatccccccccacatcctgctggccaccgcctggatctctggagagaccgtagcgat

SEQ ID NO: 54 - DNA, 724 bp, synthetic construct
  misc_feature 1..724: note = Synthetic polynucleotide
  source 1..724: mol_type = other DNA
atcgctacggtctcggctccgcaagcgacgccggcagcagcgccggcgaccccaccctgagaagaagaatcgagccctgggacttcgaggccatcttcgaccctagagagctgagaaaggaggcctgcctgctgtacgagatcaagtggggcccctgccacaagatctggagacacagcggcaagaacaccacaagacacgtggaggtgaacttcatcgagaagatcacaagcgagagacagttttgcagcagcacatcttgcagcatcatctggttcctgagctggagcccctgctgggagtgcagcaaggccatcaccgagttcctgagacaaagacccggcgtgaccctggtgatctacgtggctagactgtaccacgccatggacgagcagaacagacaaggcctgcgggacctgatcaagagcggcgtgaccattcagatcatgaccacccccgagtacgactactgctggagaaacttcgtgaactacccccccggcaaggacacccactgccccatgtacccccccctgtggatgaagctgtacgccctggagctgcactgcatcatcctgagcctgcccccctgcctgatgatcagcagaagatgtcagaagcagctgacctggtacagactgaacttgcagaactgccactatcagcagatccccccccacatcctgctggccaccgcctggatctctggagagaccgtagcgat

SEQ ID NO: 55 - skipped

SEQ ID NO: 56 - DNA, 20 bp, synthetic construct
  misc_feature 1..20: note = Synthetic nucleotide
  source 1..20: mol_type = other DNA
ttcacctgcccaaggaaaaa

SEQ ID NO: 57 - DNA, 20 bp, synthetic construct
  misc_feature 1..20: note = Synthetic nucleotide
  source 1..20: mol_type = other DNA
aagccaccccaagttagatc

SEQ ID NO: 58 - DNA, 20 bp, synthetic construct
  misc_feature 1..20: note = Synthetic nucleotide
  source 1..20: mol_type = other DNA
aacttaccccaaacaattag

SEQ ID NO: 59 - DNA, 20 bp, synthetic construct
  misc_feature 1..20: note = Synthetic nucleotide
  source 1..20: mol_type = other DNA
actcacgctggatagcctcc

SEQ ID NO: 60 - DNA, 20 bp, synthetic construct
  misc_feature 1..20: note = Synthetic nucleotide
  source 1..20: mol_type = other DNA
cttaccccacttaactatct

SEQ ID NO: 61 - DNA, 23 bp, synthetic construct
  misc_feature 1..23: note = Synthetic nucleotide
  source 1..23: mol_type = other DNA
actccagttcctgacggctgagg

SEQ ID NO: 62 - DNA, 20 bp, synthetic construct
  misc_feature 1..20: note = Synthetic nucleotide
  source 1..20: mol_type = other DNA
aattcccacagactcatggt

SEQ ID NO: 63 - DNA, 20 bp, synthetic construct
  misc_feature 1..20: note = Synthetic nucleotide
  source 1..20: mol_type = other DNA
gtgcatccagcgcttcgcac

SEQ ID NO: 64 - DNA, 20 bp, synthetic construct
  misc_feature 1..20: note = Synthetic nucleotide
  source 1..20: mol_type = other DNA
actcacgctggatagcctcc

SEQ ID NO: 65 - DNA, 20 bp, synthetic construct
  misc_feature 1..20: note = Synthetic nucleotide
  source 1..20: mol_type = other DNA
agagcaacagtgctgtggcc

SEQ ID NO: 66 - DNA, 20 bp, synthetic construct
  misc_feature 1..20: note = Synthetic nucleotide
  source 1..20: mol_type = other DNA
cccctaccatgactttattc

SEQ ID NO: 67 - DNA, 20 bp, synthetic construct
  misc_feature 1..20: note = Synthetic nucleotide
  source 1..20: mol_type = other DNA
acgacgcgtgggtggcaagc

SEQ ID NO: 68 - DNA, 20 bp, synthetic construct
  misc_feature 1..20: note = Synthetic nucleotide
  source 1..20: mol_type = other DNA
gctttggtcccattggtcgc

SEQ ID NO: 69 - DNA, 20 bp, synthetic construct
  misc_feature 1..20: note = Synthetic nucleotide
  source 1..20: mol_type = other DNA
gctactctctctttctggcc

SEQ ID NO: 70 - DNA, 20 bp, synthetic construct
  misc_feature 1..20: note = Synthetic nucleotide
  source 1..20: mol_type = other DNA
acgcaatgctcagacgcaga

SEQ ID NO: 71 - DNA, 20 bp, synthetic construct
  misc_feature 1..20: note = Synthetic nucleotide
  source 1..20: mol_type = other DNA
cgaacaaggtaaggagtctg

SEQ ID NO: 72 - DNA, 20 bp, synthetic construct
  misc_feature 1..20: note = Synthetic nucleotide
  source 1..20: mol_type = other DNA
tgagcaaaggcctggggtgc

SEQ ID NO: 73 - DNA, 20 bp, synthetic construct
  misc_feature 1..20: note = Synthetic nucleotide
  source 1..20: mol_type = other DNA
ggggtgcaggagtgctacca

SEQ ID NO: 74 - DNA, 20 bp, synthetic construct
  misc_feature 1..20: note = Synthetic nucleotide
  source 1..20: mol_type = other DNA
ggtaatggacagagttatcg

SEQ ID NO: 75 - DNA, 20 bp, synthetic construct
  misc_feature 1..20: note = Synthetic nucleotide
  source 1..20: mol_type = other DNA
gaatactacccaaatgcgta

SEQ ID NO: 76 - DNA, 20 bp, synthetic construct
  misc_feature 1..20: note = Synthetic nucleotide
  source 1..20: mol_type = other DNA
ggatccctcgtataacaata

SEQ ID NO: 77 - DNA, 20 bp, synthetic construct
  misc_feature 1..20: note = Synthetic nucleotide
  source 1..20: mol_type = other DNA
tggactacatagttgtgtga

SEQ ID NO: 78 - DNA, 20 bp, synthetic construct
  misc_feature 1..20: note = Synthetic nucleotide
  source 1..20: mol_type = other DNA
ttcaatcctcttgtcacctg

SEQ ID NO: 79 - DNA, 20 bp, synthetic construct
  misc_feature 1..20: note = Synthetic nucleotide
  source 1..20: mol_type = other DNA
cgaacaaggtaaggagcctg

SEQ ID NO: 80 - DNA, 20 bp, synthetic construct
  misc_feature 1..20: note = Synthetic nucleotide
  source 1..20: mol_type = other DNA
tcgcccttggtgttacacca

SEQ ID NO: 81 - DNA, 20 bp, synthetic construct
  misc_feature 1..20: note = Synthetic nucleotide
  source 1..20: mol_type = other DNA
tcgcccttggtgttatacca

SEQ ID NO: 82 - DNA, 20 bp, synthetic construct
  misc_feature 1..20: note = Synthetic nucleotide
  source 1..20: mol_type = other DNA
tggtagcaatcctggaccac

SEQ ID NO: 83 - DNA, 20 bp, synthetic construct
  misc_feature 1..20: note = Synthetic nucleotide
  source 1..20: mol_type = other DNA
ctgccaagcttggtcatcta

SEQ ID NO: 84 - DNA, 22 bp, synthetic construct
  misc_feature 1..22: note = Synthetic nucleotide
  source 1..22: mol_type = other DNA
gtgtcaggtaatgtgctaaaca

SEQ ID NO: 85 - DNA, 20 bp, synthetic construct
  misc_feature 1..20: note = Synthetic nucleotide
  source 1..20: mol_type = other DNA
tctgcttctccagccctggc

SEQ ID NO: 86 - DNA, 20 bp, synthetic construct
  misc_feature 1..20: note = Synthetic nucleotide
  source 1..20: mol_type = other DNA
gatgttccaatcagtacgca

SEQ ID NO: 87 - protein, 229 aa, Rattus norvegicus [sequence omitted]

SEQ ID NO: 88 - DNA, 684 bp, synthetic construct
  source 1..684: mol_type = other DNA
tcctcagagactgggcctgtcgccgtcgatccaaccctgcgccgccggattgaacctcacgagtttgaagtgttctttgaccctcgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcccaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaag

SEQ ID NO: 89 - protein, 236 aa, Sus scrofa
MASDRGPSAGDATSRRRIEPWEFEVFFDPRELRKETCLLYELQWGRSRDTWRHTGKNTTNHVERNFLAKITSERHFHPSVHCSIVWFLSWSPCWECSEAIREFLDQHPSVTLVIYVARLFQHMDPQNRQGLRDLVNHGVTIQIMGAPEYDYCWRNFVNYPPGKEAHWPRFPPVWMTLYALELHCIILGLPPCLKISRRCQNQLTFFRLTLQNCHYQTIPPHILLATGLIQLPVIYR

SEQ ID NO: 90 - DNA, 705 bp, synthetic construct
  source 1..705: mol_type = other DNA
gcaagcgacagaggccctagcgccggcgacgccacaagcagaagaagaatcgagccctgggagttcgaggtgttcttcgaccctagagagctgagaaaggaaacctgcctgctgtacgagttgcagtggggcagaagcagagacacctggagacacaccggcaagaacaccaccaaccacgtggagagaaacttcctggccaagatcacaagcgagagacacttccatccgagcgtgcattgcagcatcgtgtggttcctgagctggagcccctgctgggagtgcagcgaggccatcagagagttcctggatcagcatcctagcgtgaccctggtgatctacgtggctagactgtttcagcacatggaccctcagaacagacaaggcctgcgggacctggtgaaccacggcgtgaccattcagatcatgggcgcccccgagtacgactactgctggagaaacttcgtgaactacccccccggcaaggaggcccactggcctagattcccccccgtgtggatgaccctgtacgccctggagctgcactgcatcatcctgggcctgcccccctgcctgaagatcagcagaagatgtcagaatcagctgaccttcttcagactgaccttgcagaactgccactatcagaccatccccccccacatcctgctggccaccggcctgattcagctgcccgtgatctacaga

(19) United States
(12) Patent Application Publication
(10) Pub. No.: US 2026/0250650 A1
(43) Pub. Date: Aug. 27, 2026

(54) BASE EDITING PROTEINS AND USES THEREOF

(71) Applicant: CRISPR THERAPEUTICS AG, Zug (CH)

(72) Inventors: John KULMAN, South Boston, MA (US); Daniel FERULLO, South Boston, MA (US); Brian FOCHTMAN, South Boston, MA (US); Aishwarya VISHWANATHAN, South Boston, MA (US); Vasudevan ACHUTHAN, South Boston, MA (US)

(21) Appl. No.: 18/877,212

(22) PCT Filed: Jun. 20, 2023
(86) PCT No.: PCT/IB2023/056337

§ 371 Date: Dec. 19, 2024

Related U.S. Application Data
(60) Provisional application No. 63/353,805, filed on Jun. 20, 2022.
(60) Provisional application No. 63/371,487, filed on Aug. 15, 2022.

(51) Int. Cl. C12N 9/22; A61K 31/7105; A61K 38/46; A61K 38/50; A61K 40/11; A61K 40/31; A61K 40/42; A61P 35/00; C07K 16/28; C12N 5/0783; C12N 9/78; C12N 15/11; C12N 15/90

(52) U.S. Cl. CPC C12N 9/226; A61K 31/7105; A61K 38/465; A61K 38/50; A61K 40/11; A61K 40/31; A61K 40/4224; A61P 35/00; C07K 16/2875; C12N 5/0636; C12N 9/78; C12N 15/11; C12N 15/907; C12Y 305/04005; C07K 2319/09; C07K 2319/70; C12N 2310/20; C12N 2510/00

(57) ABSTRACT

The present disclosure provides base editing fusion proteins, protein complexes, compositions, cells and methods of using the same for targeted nucleic acid editing. The base editing fusion protein can comprise a Cas9 domain and a deaminase domain capable of deaminating a nucleotide base in a target nucleic acid sequence.

### CROSS-REFERENCE TO RELATED APPLICATIONS

This application is a U.S. national phase application under 35 U.S.C. § 371 of International Application No. PCT/IB2023/056337, filed on Jun. 20, 2023 and published as WO 2023/248110 A2 on Dec. 28, 2023, which claims the benefit under 35 U.S.C. § 119 (e) to U.S. Provisional Patent Application No. 63/353,805, filed on Jun. 20, 2022, and U.S. Provisional Patent Application No. 63/371,487, filed on Aug. 15, 2022; the content of each of these related applications is incorporated herein by reference in its entirety for all purposes.

### REFERENCE TO SEQUENCE LISTING

The present application is being filed along with a Sequence Listing in electronic format. The Sequence Listing is provided as a file entitled 80EM-341720-US_SequenceListing, created Jun. 19, 2023, which is 126 kilobytes in size. The information in the electronic format of the Sequence Listing is incorporated herein by reference in its entirety.

### BACKGROUND

### Field

The present disclosure generally relates to the field of molecular biology and biotechnology, including gene editing.

### Description of the Related Art

CRISPR (Clustered Regularly Interspaced Short Palindromic Repeats)-Cas (CRISPR associated) systems have been used for genome editing, and require a Cas polypeptide or variant thereof guided by a customizable guide RNA (gRNA) for programmable DNA targeting. Genome editing technologies involving non-homologous end-joining (NHEJ) and homology-directed repair (HDR) can lead to gene disruption through the introduction of insertions, deletions, translocations or other DNA rearrangements at the site of a double-stranded DNA break (DSB).

There is a need for a specific and precise genome editing strategy without generating undesired byproducts.

### SUMMARY

Disclosed herein include a fusion protein, in some embodiments, comprises: a Cas9 domain, wherein the Cas9 domain when associated with a guide RNA (gRNA) specifically binds to a target nucleic acid sequence; and a cytidine deaminase domain capable of deaminating a cytosine base in a single-stranded portion of the target nucleic acid sequence.

The cytidine deaminase domain can comprise, for example, a deaminase from an apolipoprotein B mRNA-editing complex (APOBEC) family deaminase. The APOBEC family deaminase can be, for example, an APOBEC1 deaminase. In some embodiments, the cytidine deaminase domain comprises a mammalian deaminase or a variant thereof. The cytidine deaminase domain can comprise a rat deaminase, an armadillo deaminase, a bat deaminase, or a variant thereof. In some embodiments, the cytidine deaminase domain comprises a deaminase from Dasypus novemcinctus, Meriones unguiculatus, Myotis lucifugus, or a variant thereof. In some embodiments, the cytidine deaminase domain comprises (1) an amino acid sequence having at least 85%, 90%, 95%, 98%, or 99% identity to any one of SEQ ID NOs: 1-18, or (2) an amino acid sequence having one, two, three, four, five, six, seven, eight, nine, or ten mismatches relative to any one of SEQ ID NOs: 1-18. In some embodiments, the cytidine deaminase domain comprises amino acid mutation(s) at one or more positions functionally equivalent to R30, E31, L32, R33, K34, E35, T36, R52, Q56, N57, N59, K60, H61, V62, L88, S89, W90, R118, Y120, H121, H122, R126, R128, R169, 1195, R197, R198, K199, Q200, P201, Q202, and L203 in the deaminase of SEQ ID NO: 1. The amino acid mutation(s) can, for example, comprise one or more amino acid substitutions of R33A, K34A, W90Y, H126E and H122A. In some embodiments, the cytidine deaminase domain does not comprise amino acid mutation(s) at one or more positions functionally equivalent to R33, K34, T36, R52, H53, Q56, K60, V62, R118, R126, R128, R169, and R198 in the deaminase of SEQ ID NO: 1. In some embodiments, the cytidine deaminase comprises an amino acid sequence selected from the group consisting of SEQ ID NOs: 1-18. In some embodiments, the cytidine deaminase is encoded by (1) a nucleic acid sequence having at least 85%, 90%, 95%, 98%, or 99% identity to any one of SEQ ID NOs: 19-36; or (2) a nucleic acid sequence having one, two, three, four, five, six, seven, eight, nine, or ten mismatches relative to any one of SEQ ID NOs: 19-36. In some embodiments, the cytidine deaminase is encoded by a nucleic acid sequence selected from the group consisting of SEQ ID NOs: 19-36.

The cytidine deaminase domain can be fused to the N-terminus or C-terminus of the Cas9 domain. In some embodiments, the Cas9 domain is fused to the cytidine deaminase domain directly or via a linker. In some embodiments, the Cas9 domain of is a nuclease-inactive Cas9, a dead Cas9, a Cas9 nickase, or a fragment or a variant thereof. The Cas9 domain can comprise or can be, for example, Streptococcus pyogenes (SpyCas9), Staphylococcus lugdunensis (SluCas9), or derived from Staphylococcus aureus (SaCas9), Neisseria meningitides Cas9, Streptococcus thermophilus Cas9, Treponema denticola Cas9, Campylobacter jejuni Cas9, S. schleiferi Cas9, or a variant thereof.

In some embodiments, the fusion protein comprises an uracil glycosylase inhibitor (UGI) domain, wherein the UGI domain inhibits a uracil-DNA glycosylase. In some embodiments, the UGI domain is fused to the N-terminus or C-terminus of the Cas9 domain. In some embodiments, the UGI domain is fused to the N-terminus or C-terminus of the deaminase domain. In some embodiments, the UGI domain is fused to the deaminase domain and/or the Cas9 domain directly or via a linker. The fusion protein can, for example, comprise two UGI domains. The fusion protein, in some embodiments, further comprises a nuclear localization sequence.

Disclosed herein includes a nucleic acid sequence comprising a sequence encoding any fusion protein disclosed herein. In some embodiments, the nucleic acid sequence comprises a sequence encoding the cytidine deaminase domain having at least 85%, 90%, 95%, 98%, or 99% identity to any one of SEQ ID NOs: 19-36.

Disclosed herein includes a complex comprising any fusion protein disclosed herein and a guide RNA bound to the Cas9 domain of the fusion protein. In some embodiments, the gRNA is a single-guide RNA (sgRNA). In some embodiments, gRNA comprises a space sequence of any one of SEQ ID NOs: 61-83.

Disclosed herein includes a composition, comprising: (a) any of the fusion protein disclosed herein or a nucleic acid sequence encoding the fusion protein disclosed herein; and (b) one or more guide RNAs targeting one or more target genes.

Disclosed herein includes a pharmaceutical composition, comprising any of the compositions disclosed herein, and one or more pharmaceutical acceptable carriers or excipients.

Disclosed herein includes a method for producing an engineered cell, comprising providing a plurality of cells; delivering to the plurality of cells (a) a fusion protein disclosed herein or a nucleic acid encoding the fusion protein and (b) at least one guide RNA targeting at least one target gene; genetically editing the at least one target gene; and producing one or more genetically engineered cells having at least one gene edit in the at least one target gene. In some embodiments, the plurality of cells are T cells or precursor cells thereof. In some embodiments, the method comprises delivering to the plurality of cells a nucleic acid encoding a chimeric antigen receptor (CAR). In some embodiments, the at least one guide RNA comprises two or more guide RNAs targeting two or more target genes and wherein the produced one or more genetically engineered cells have two or more gene edits in the two or more target genes. In some embodiments, the at least one target gene is selected from the Regnase-1 (Reg1) gene, the Transforming Growth Factor Beta Receptor II (TGFBRII) gene, the TRAC gene, the beta-2-microglobulin (62M) gene, the CD70 gene, T cell receptor alpha chain constant region (TRAC) gene, or a combination thereof.

Disclosed herein includes a population of genetically engineered cells, which is prepared by a method disclosed herein. In some embodiments, the population of genetically engineered cells comprise at least two gene edits. In some embodiments, at least 50% of the cells in the population of genetically engineered cells comprise at least two genome edits and wherein fewer than 1%, 0.5%, 0.2% or 0.1% of the cells in the population of genetically engineered cells have an insertion, deletion, translocation or other DNA rearrangement. In some embodiments, the cells are T cells.

Disclosed herein includes a method, comprising administering to a subject a population of genetically engineered cells disclosed herein. In some embodiments, the subject is a human subject. In some embodiments, the subject has a disease or disorder.

Disclosed herein includes a method of DNA editing, comprising contacting a DNA molecule with a fusion protein disclosed herein; and a guide RNA (gRNA) targeting a target nucleotide sequence of the DNA molecule, thereby editing the DNA molecule by deaminating a nucleotide base within the target nucleotide sequence of the DNA molecule. In some embodiments, the target nucleotide sequence of the DNA molecule is associated with the disease or disorder. In some embodiments, the deamination corrects a point mutation in the target nucleotide sequence associated with the disease or disorder. In some embodiments, the target nucleotide sequence encodes a protein and wherein the deamination results in a reduction or inhibition of the expression level of the encoded protein. In some embodiments, the target nucleotide sequence comprises a T to C point mutation associated with a disease or disorder and wherein the deamination of the mutant C base results in a sequence that is not associated with the disease or disorder. In some embodiments, the gRNA targets a target nucleotide sequence of a LPA gene. In some embodiments, the contacting is in vivo in a subject suspected to have, having or diagnosed with the disease or disorder.

Disclosed herein includes a method of preventing or treating a disease or disorder in a subject in need thereof, comprising: administering to the subject a therapeutically effective amount of a composition disclosed herein or the pharmaceutical composition disclosed herein, wherein the one or more target gene is associated with the disease or disorder. The disease or disorder can be cancer. The cancer can be, for example, pancreatic cancer, gastric cancer, ovarian cancer, uterine cancer, breast cancer, prostate cancer, testicular cancer, thyroid cancer, nasopharyngeal cancer, non-small cell lung (NSCLC), glioblastoma, neuronal, soft tissue sarcomas, leukemia, lymphoma, melanoma, colon cancer, colon adenocarcinoma, brain glioblastoma, hepatocellular carcinoma, liver hepatocholangiocarcinoma, osteosarcoma, gastric cancer, esophagus squamous cell carcinoma, advanced stage pancreas cancer, lung adenocarcinoma, lung squamous cell carcinoma, lung small cell cancer, renal carcinoma, intrahepatic biliary cancer, or a combination thereof. In some embodiments, the disease or disorder is calcific aortic valve disease, myocardial infarctions, coronary heart disease, atherosclerosis, thrombosis, stroke, coronary artery disease, familial hyperlipidemia, myocardial infarction, peripheral arterial disease, calcific aortic valve stenosis, or a combination thereof. In some embodiments, the disease or disorder is a metabolic disease, a lipid metabolism disease, obesity, atherosclerosis, hyperfattyacidemia, metabolic syndrome, dyslipidemia, hypobetalipoproteinemia, familial hypercholesterolemia (including homozygous familial hypercholesterolemia (HoFH) and heterozygous familial hypercholesterolemia (HeFH)), hypertriglyceridemia, familial combined hyperlipidemia, familial chylomicronemia syndrome, multifactorial chylomicronemia syndrome, familial combined hyperlipidemia (FCHL), metabolic syndrome (MetS), nonalcoholic fatty liver disease (NAFLD), elevated lipoprotein (a), cholesterol, and/or triglyceride in the blood, or a combination thereof.

In some embodiments, the one or more target gene comprises a LPA gene. In some embodiments, the one or more target gene comprises an ANGPTL3 gene. In some embodiments, the one or more target gene comprise the Reg1 gene, the TGFBRII gene, the TRAC gene, the B2M gene, the TRAC gene, or a combination thereof.

### BRIEF DESCRIPTION OF THE DRAWINGS

FIG. 1 shows the degree of B2M and FAS knockdown by sgRNA using seven BE4 cytosine base editor (CBE) mRNAs in Jurkat cells (determined by flow cytometry).

FIGS. 2A-2B show on-target activity based on Amp-Seq. FIG. 2A is a diagram showing the percentage of knockdown from flow cytometry by deaminase enzymes from various species. FIG. 2B is a diagram showing the percentage of cytidine (C) to thymine (T) conversion rate by deaminases from various species. The C to T conversion rates correlate well with the percentage of protein loss.

FIGS. 3A-3B depict diagrams showing the off-target activity based on Amp-Seq results from the R-loop assay. FIGS. 3A-3B show C to T conversion rate by deaminases from various species. Higher on-target C→T rates with Rat & Orangutan APOBEC1 (~70%) were observed. Also observed were: off-target C→T rates with Rat APOBEC1 and Doman 5 at about 15-20%; Rat APOBEC1 and Doman 6 at about 10-15%.

FIG. 4 depicts a diagram showing the sum of C to T conversion rates across the on-target spacer regions and sum of C to T off-target activity across the R-loop amplicons by deaminases from various species. Deaminase from armadillo (e.g., nine-banded armadillo), orangutan, rat, bat (e.g., little brown rat), and Mongolian gerbil exhibit high on-target editing efficiency and low off-target editing efficiency.

FIG. 5 depicts a plot showing that RNP & BE CTX131 CAR-T Cells achieved comparable editing efficiencies. About 70% CAR insertion, greater than 98% TCR & CD70 KO, about 70% B2M1 RNP editing and showing superior B2M1 editing by base editor (BE).

FIG. 6 depicts a plot showing absence of indels in base edited CTX131 CAR T cells.

FIGS. 7A-7B and FIGS. 8A-8B show base edited CTX131 CAR T cell health. Equivalent proliferative capacity of BE & RNP CTX131 were observed. CAR T Cell viability was improved with Modified CBE mRNA. Similar CD4/CD8 ratios for BE & RNP CTX131, and there was no change cryopreserve d10 post HDR.

FIG. 9 depicts a graph showing RNP and BE anti-CD70 CAR-Ts are comparably efficacious in vitro.

FIGS. 10A-10B depict plots showing that base edited CAR-T cells are high in vivo efficacy. Group 2: CTX131-RNP, and Group 4: CTX131-BE.

FIG. 11 depicts targeting of regions in the Kringle IV-2 repeats of the LPA gene by sgRNAs to introduce stop codons.

FIGS. 12A-12D depict graphs showing base editing in human liver cell lines using CBE mRNAs comprising rat APOBEC1 deaminase or orangutan deaminase. FIG. 11B shows base editing in iPSC-derived hepatoblasts. pmSTOP-Premature Stop Codon, sa/sd=Splice Acceptor/Donor Defect.

FIGS. 13A-13B depict graphs showing base editing efficiency in NHP primary liver cells using CBE mRNAs comprising rat APOBEC1 deaminase or orangutan deaminase.

FIG. 14A shows % base editing in human T cells using CBEs comprising deaminases from rat orangutan, and armadillo, respectively. FIG. 14B shows on-target vs. off-target spurious deamination by the same three CBEs.

FIG. 15 shows a sequence alignment of rat APOBEC1 (rAPOBEC1), wildtype deaminase from Nine-banded armadillo (SEQ ID NO: 1), wildtype deaminase from Mongolian gerbil (SEQ ID NO: 7), and wildtype deaminase from Little brown bat (SEQ ID NO: 13). Row 5 of the sequence alignment shows a sequence of an exemplary homology model of rAPOBEC1. Amino acids at various positions were selected based on proximity to modeled ssDNA strand for making mutant deaminases (including the residues colored in purple, e.g., one or more amino acids of RELRKET (positions 30-36), R at position 52, QN (positions 56 and 57), NKHV (positions 59-62), LSW (positions 88-90), R at position 118, YHH at positions 120-122, R at position 126, R at position 128, R at position 169, I at position 195, R at position 197, R at position 198, and KQPQL at positions 199-203). Residues colored in other colors can also be mutated (e.g., substitution, deletion, insertion) individually or in combination of any other mutations.

FIG. 16 illustrates a modified R-loop assay used for evaluating spurious off-target deamination. The modified R-loop assay eliminates the need for using nSaCas9-2XUGI mRNA. Top panel illustrates the process used for generating the cell lines stably expressing the nSaCas9-2XUGI protein using lentiviruses encoding nSaCas9-2XUGI-P2A-Hygromycin resistance. Cells expressing nSaCas9-2XUGI were selected using Hygromycin post lentivirus transduction. The modified R-loop assay can avoid variability between R-loop assay experiments, as pan-cellular expression of nSaCas9-2XUGI was observed in the selected population.

FIG. 17A is a table showing C to T base editing efficiencies for Group A knockout (FAS, TRAC, B2M, CD70, and optionally REG1) and Group B knockout (FAS, TRAC, B2M, CD70, and optionally RFX5). FIG. 17B-C are graphically representation of the five-plex editing data of Group A (FIG. 17B) and Group B (FIG. 17C).

FIG. 18 illustrates an exemplary experiment layout used in multiplex editing optimization disclosed herein.

FIG. 19A depicts a schematic representation of a deaminase homology model constructed from homology modeling. FIG. 19B shows an alignment of potential sites for mutations identified by different molecular modeling programs.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include base editing fusion proteins, nuclei acid sequences, compositions, engineered cells, kits and method of using the same for target nucleic acid editing. In some embodiments, a base editing fusion protein comprises a Cas9 domain, wherein the Cas9 domain when associated with a guide RNA (gRNA) specifically binds to a target nucleic acid sequence, and a deaminase domain (e.g., a cytidine deaminase domain) capable of deaminating a nucleotide base (e.g., from C to U) in a target nucleic acid sequence.

### Definition

As used herein, the term “about” means plus or minus 5% of the provided value.

As used herein, the term “gene editing” (including genomic editing) is a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When an sequence of an endogenous gene is edited, for example by deletion, insertion or substitution of nucleotide(s)/nucleic acid(s), the endogenous gene comprising the affected sequence can be knocked-out or knocked-down due to the sequence alteration. Therefore, targeted editing can be used to disrupt endogenous gene expression.

As used herein, a “CRISPR-Cas9” system is a naturally-occurring defense mechanism in prokaryotes that has been repurposed as a RNA-guided DNA-targeting platform used for gene editing. It relies on the DNA nuclease Cas9, and two noncoding RNAs-crisprRNA (crRNA) and trans-activating RNA (tracrRNA) to target the cleavage of DNA. crRNA drives sequence recognition and specificity of the CRISPR-Cas9 complex through Watson-Crick base pairing typically with a 20 nucleotide (nt) sequence in the target DNA. The CRISPR-Cas9 complex only binds DNA sequences that contain a sequence match to the first 20 nt of the crRNA, single-guide RNA (sgRNA), if the target sequence is followed by a specific short DNA motif (with the sequence NGG) referred to as a protospacer adjacent motif (PAM). TracrRNA hybridizes with the 3′ end of crRNA to form an RNA-duplex structure that is bound by the Cas9 endonuclease to form the catalytically active CRISPR-Cas9 complex, which can then cleave the target DNA. Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end). After binding of CRISPR-Cas9 complex to DNA at a specific target site and formation of the site-specific DSB, the next key step is repair of the DSB. Cells use two main DNA repair pathways to repair the DSB: non-homologous end-joining (NHEJ) and homology-directed repair (HDR). In some embodiments, CRISPR-Cas9 gene editing system comprises an RNA-guided nuclease and one or more guide RNAs targeting one or more target genes.

As used herein, the term “RNA-guided endonuclease” refers to a polypeptide capable of binding a RNA (e.g., a gRNA) to form a complex targeted to a specific DNA sequence (e.g., in a target DNA). A non-limiting example of RNA-guided endonuclease is a Cas polypeptide (e.g., a Cas endonuclease, such as a Cas9 endonuclease). In some embodiments, the RNA-guided endonuclease as described herein is targeted to a specific DNA sequence in a target DNA by an RNA molecule to which it is bound. The RNA molecule can include a sequence that is complementary to and capable of hybridizing with a target sequence within the target DNA, thus allowing for targeting of the bound polypeptide to a specific location within the target DNA.

As used herein, the term “guide RNA” or “gRNA” refers to a site-specific targeting RNA that can bind an RNA-guided endonuclease to form a complex, and direct the activities of the bound RNA-guided endonuclease (such as a Cas endonuclease) to a specific target sequence within a target nucleic acid. The guide RNA can include one or more RNA molecules.

Unless otherwise indicated “nuclease” and “endonuclease” are used interchangeably herein to refer to an enzyme which possesses endonucleolytic catalytic activity for polynucleotide cleavage.

As used herein, the term “Cas endonuclease” or “Cas nuclease” refers to an RNA-guided DNA endonuclease associated with the CRISPR adaptive immunity system.

The term “deaminase” refers to an enzyme that catalyzes a deamination reaction. In some embodiments, the deaminase is a cytidine deaminase, catalyzing the hydrolytic deamination of cytidine or deoxycytidine to uracil or deoxyuracil, respectively.

As used herein, the term “invariable region” of a gRNA refers to the nucleotide sequence of the gRNA that associates with the RNA-guided endonuclease. In some embodiments, the gRNA comprises a crRNA and a transactivating crRNA (tracrRNA), wherein the crRNA and tracrRNA hybridize to each other to form a duplex. In some embodiments, the crRNA comprises 5′ to 3′: a spacer sequence and minimum CRISPR repeat sequence (also referred to as a “crRNA repeat sequence” herein); and the tracrRNA comprises a minimum tracrRNA sequence complementary to the minimum CRISPR repeat sequence (also referred to as a “tracrRNA anti-repeat sequence” herein) and a 3′ tracrRNA sequence. In some embodiments, the invariable region of the gRNA refers to the portion of the crRNA that is the minimum CRISPR repeat sequence and the tracrRNA.

As used herein, the term “target DNA” refers to a DNA that includes a “target site” or “target sequence.” The term “target sequence” is used herein to refer to a nucleic acid sequence present in a target DNA to which a DNA-targeting sequence or segment (also referred to herein as a “spacer”) of a gRNA can hybridize, provided sufficient conditions for hybridization exist. For example, the target sequence 5′-GAGCATATC-3′ within a target DNA is targeted by (or is capable of hybridizing with, or is complementary to) the RNA sequence 5′-GAUAUGCUC-3′. Hybridization between the DNA-targeting sequence or segment of a gRNA and the target sequence can, for example, be based on Watson-Crick base pairing rules, which enables programmability in the DNA-targeting sequence or segment. The DNA-targeting sequence or segment of a gRNA can be designed, for instance, to hybridize with any target sequence.

As used herein, the terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases (e.g., the five biologically occurring bases adenine, guanine, thymine, cytosine and uracil) or other natural, chemically or biochemically modified, non-natural, or derivatized nucleotide bases. In some embodiments, a nucleic acid or polynucleotide can refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages.

As used herein, a “secondary structure” of a nucleic acid molecule (e.g., an RNA fragment, or a gRNA) refers to the base pairing interactions within the nucleic acid molecule.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

As used herein, “sequence identity” or “identity” in the context of two nucleic acid or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

As used herein, the term “fusion protein” refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a recombinase. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein can be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

As used herein, the term “binding” refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions can be characterized by a dissociation constant (Kd), for example a Kd of, or a Kd less than, 10⁻⁶ M, 10⁻⁷ M, 10⁻⁸ M, 10⁻⁹M, 10⁻¹⁰ M, 10⁻¹¹ M, 10⁻¹² M, 10⁻¹³ M, 10⁻¹⁴ M, 10⁻¹⁵ M, or a number or a range between any two of these values. Kd can be dependent on environmental conditions, e.g., pH and temperature. “Affinity” refers to the strength of binding, and increased binding affinity is correlated with a lower Kd.

As used herein, the term “hybridizing” or “hybridize” refers to the pairing of substantially complementary or complementary nucleic acid sequences within two different molecules. Pairing can be achieved by any process in which a nucleic acid sequence joins with a substantially or fully complementary sequence through base pairing to form a hybridization complex. “Hybridizing” or “hybridize” can comprise denaturing the molecules to disrupt the intramolecular structure(s) (e.g., secondary structure(s)) in the molecule. In some embodiments, denaturing the molecules comprises heating a solution comprising the molecules to a temperature sufficient to disrupt the intramolecular structures of the molecules. In some instances, denaturing the molecules comprises adjusting the pH of a solution comprising the molecules to a pH sufficient to disrupt the intramolecular structures of the molecules. For purposes of hybridization, two nucleic acid sequences or segments of sequences are “substantially complementary” if at least 80% of their individual bases are complementary to one another. In some embodiments, a splint oligonucleotide sequence is not more than about 50% identical to one of the two polynucleotides (e.g., RNA fragments) to which it is designed to be complementary. The complementary portion of each sequence can be referred to herein as a ‘segment’, and the segments are substantially complementary if they have 80% or greater identity.

The terms “complementarity” and “complementary” mean that a nucleic acid can form hydrogen bond(s) with another nucleic acid based on traditional Watson-Crick base paring rule, that is, adenine (A) pairs with thymine (U) and guanine (G) pairs with cytosine (C). Complementarity can be perfect (e.g. complete complementarity) or imperfect (e.g. partial complementarity). Perfect or complete complementarity indicates that each and every nucleic acid base of one strand is capable of forming hydrogen bonds according to Watson-Crick canonical base pairing with a corresponding base in another, antiparallel nucleic acid sequence. Partial complementarity indicates that only a percentage of the contiguous residues of a nucleic acid sequence can form Watson-Crick base pairing with the same number of contiguous residues in another, antiparallel nucleic acid sequence. In some embodiments, the complementarity can be at least 70%, 80%, 90%, 100% or a number or a range between any two of these values. In some embodiments, the complementarity is perfect, i.e. 100%. For example, the complementary candidate sequence segment is perfectly complementary to the candidate sequence segment, whose sequence can be deducted from the candidate sequence segment using the Watson-Crick base pairing rules.

As used herein, the term “vector” refers to a polynucleotide construct, typically a plasmid or a virus, used to transmit genetic material to a host cell. Vectors can be, for example, viruses, plasmids, cosmids, or phage. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the terms “transfection” or “infection” refer to the introduction of a nucleic acid into a host cell, such as by contacting the cell with a recombinant MVA virus or a gutless picornaviral particle as described herein.

As used herein, the term “transgene” refers to any nucleotide or DNA sequence that is integrated into one or more chromosomes of a target cell by human intervention. In some embodiment, the transgene comprises a polynucleotide that encodes a protein of interest. The protein-encoding polynucleotide is generally operatively linked to other sequences that are useful for obtaining the desired expression of the gene of interest, such as transcriptional regulatory sequences. In some embodiments, the transgene can additionally comprise a nucleic acid or other molecule(s) that is used to mark the chromosome where it has integrated.

As used herein, the term “prophylaxis,” “prevent,” “preventing,” “prevention,” and grammatical variations thereof as used herein refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented. A treatment is considered “effective treatment,” if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

As used herein, the terms “effective amount” or “pharmaceutically effective amount” or “therapeutically effective amount” refer to an amount sufficient to effect beneficial or desirable biological and/or clinical results. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The term “pharmaceutically acceptable excipient” as used herein refers to any suitable substance that provides a pharmaceutically acceptable carrier, additive or diluent for administration of a compound(s) of interest to a subject. Pharmaceutically acceptable excipient can encompass substances referred to as pharmaceutically acceptable diluents, pharmaceutically acceptable additives, and pharmaceutically acceptable carriers.

As used herein, a “subject” refers to an animal for whom a diagnosis, treatment, or therapy is desired. I some embodiments, the subject is a mammal. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a primate. In some embodiments, the mammal is a human. In some embodiments, the mammal is not a human.

The wording “associated with” as used herein with reference to two items indicates a relation between the two items such that the occurrence of a first item is accompanied by the occurrence of the second item, which includes but is not limited to a cause-effect relation and sign/symptoms-disease relation.

### Base Editing

RNA-guided nucleases from CRISPR systems generate precise breaks in DNA or RNA at specified positions. In cells, this activity can lead to changes in DNA sequence or RNA transcript abundance. Base editing is a genome editing approach that uses components from CRISPR systems together with DNA editing enzymes, such as deaminase enzymes, to directly install point mutations into cellular DNA or RNA by converting one base or base pair into another, while minimizing the formation of double-stranded DNA breaks (DSBs).

Provided herein include a base editing fusion protein, a protein complex comprising the fusion protein and a bound guide RNA, and methods of using the fusion protein and protein complex for targeted base editing of nucleic acids. The fusion protein described herein can comprise a Cas9 domain capable of binding to a guide RNA (gRNA) which in turn binds a target nucleic acid sequence of a nucleic acid via hybridization; and a deaminase domain that can deaminate a nucleobase, such as, cytidine. The deamination of a nucleobase by a deaminase can lead to a point mutation at the respective residue (e.g., from cytosine to uracil), which is referred to herein as base editing or nucleic acid editing. Fusion proteins comprising a Cas9 variant or domain and a deaminase domain can thus be used for the targeted editing of nucleic acid sequences. Such fusion proteins are useful for targeted editing of DNA in vitro, e.g., for the generation of mutant cells or animals; for the introduction of targeted mutations, e.g., for the correction of genetic defects in cells ex vivo, e.g., in cells obtained from a subject that are subsequently re-introduced into the same or another subject; and for the introduction of targeted mutations in vivo, e.g., the correction of genetic defects or the introduction of deactivating mutations in disease-associated genes in a subject.

The nucleobase editors or base editors (BEs) described herein comprise fusions between a catalytically impaired Cas nuclease and a base-modification enzyme that operates on single-stranded DNA (ssDNA). Upon binding to its target locus in a DNA molecule, base pairing between the gRNA and target DNA strand leads to displacement of a small segment of single-stranded DNA in an “R-loop”. DNA bases within this single-stranded DNA bubble are modified by the deaminase enzyme. In some embodiments, the fusion protein comprises a nuclease-inactive Cas9 (dCas9) fused to a deaminase. In some embodiments, the fusion protein comprises a Cas9 nickase fused to a deaminase. In some embodiments, the fusion protein comprises a Cas9 nickase fused to a deaminase and further fused to a uracil glycosylase inhibitor (UGI) domain. In some embodiments, the base editing fusion proteins described herein exhibit enhanced on-target editing efficiency and reduced off-target editing efficiency. In some embodiments, the on-target editing efficiency is at least 50%, 60%, 70%, 80%, 90%, 95% or greater. In some embodiments, the off-target editing is reduced by about, at least or at least about 80%, 85%, 90%, 95%, 98%, 99% or 100%.

### Deaminase Domain

The base editing fusion protein described herein can comprise a nucleic acid editing domain such as a deaminase or deaminase domain. The term “deaminase” refers to an enzyme that catalyzes a deamination reaction. In some embodiments, the deaminase or deaminase domain belongs to the deaminase superfamily. The deaminase superfamily encompasses zinc-dependent enzymes catalyzing the deamination of bases in free nucleotides and nucleic acids. The deaminase superfamily displays a conserved β-sheet with five β-strands arranged in 2-1-3-4-5 order interleaved with three α-helices forming an α/β-fold. The active site comprises two zinc-chelating motifs, respectively represented by a motif of H×E/C×E/D×E at the end of helix 2 and CxₙC (where x is any amino acid and n is >2) located in loop 5 and the beginning of helix 3. In some embodiments, the zinc ion is coordinated by the side chains of residues His and Cys, such as His257, Cys291 and Cys288 in APOBEC3G.

In some embodiments, the deaminase is a cytidine deaminase, catalyzing the hydrolytic deamination of cytidine or deoxycytidine to uracil or deoxyuracil, respectively. In some embodiments, the deaminase is an apolipoprotein B mRNA-editing complex (APOBEC) family deaminase. In some embodiments, an APOBEC family deaminase contains a conserved α/β-fold core domain at the N-terminal and a C-terminal domain. The APOBEC deaminase core domain comprises three active loops (e.g., loops 1, 3 and 7 in APOBEC1 and loops 1, 5 and 7 in APOBEC3) containing amino acid residues known to form interactions with a nucleic acid upon its binding to the deaminase. In some embodiments, the C-terminal domain of an APOBEC family deaminase comprises a β-hairpin and three small helical domains. In some embodiments, the deaminase herein described is a dimer formed by two APOBEC deaminase monomers mediated through interactions between the C-terminal domains. In some embodiments, amino acid residues in the active loops of the deaminase core domain and/or residues in the C-terminal domain can be mutated to generate deaminase variants with desired properties such as low spurious off-target editing activity and improved on-target editing precision (e.g., by narrowing editing window). The APOBEC family of cytosine deaminase enzymes encompasses eleven proteins that serve to initiate mutagenesis in a controlled and beneficial manner as will be understood by a person skilled in the art.

In some embodiments, the deaminase is an APOBEC1 family deaminase. In some embodiments, the deaminase is an APOBEC3 family deaminase. In some embodiments, the deaminase is an activation-induced cytidine deaminase (AID), which is responsible for the maturation of antibodies by converting cytosines in ssDNA to uracils in a transcription-dependent, strand-biased fashion. In some embodiments, the deaminase is an ACF1/ASE deaminase. Additional suitable nucleic acid-editing enzymes or domains will be apparent to the skilled artisan based on this disclosure.

The deaminase or deaminase domain can comprise, or can be, a naturally-occurring deaminase from an organism, mammals, fungus, reptiles, amphibians and birds. In some embodiments, the deaminase or deaminase domain is a variant of a naturally-occurring deaminase from an organism, that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism. In some embodiments, the deaminase or deaminase domain is a variant of a naturally-occurring deaminase comprising one or more mutations or a deletion or insertion of one or more residues within a sequence. In some embodiments, the one or more mutations/deletions/insertions result in altered catalytic deaminase activity. For example, the one or more mutations/deletions/insertions result in reduced catalytic deaminase activity such that the deaminase or deaminase domain is less likely to catalyze the deamination of a residue adjacent to a target residue, thereby narrowing the deamination window. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)).

The deaminase or deaminase domain can be from a mammal, for example, rat, bat, armadillo, or orangutan; or be a variant thereof. In some embodiments, the deaminase or deaminase domain is a rat deaminase. In some embodiments, the deaminase or deaminase domain is a bat deaminase. In some embodiments, the deaminase or deaminase domain is an armadillo deaminase. In some embodiments, the deaminase or deaminase domain is an orangutan deaminase. In some embodiments, the deaminase or deaminase domain is from Dasypus novemcinctus (nine-banded armadillo), Meriones unguiculatus (Mongolian gerbil) or Myotis lucifugus (little brown bat). In some embodiments, the deaminase or deaminase domain is from Dasypus novemcinctus.

The deaminase can be, for example, a deaminase (SEQ ID NO: 1) from Nine-banded Armadillo (Dasypus novemcinctus) encoded by a nucleic acid sequence of SEQ ID NO: 19. The residues in bold and underlined in SEQ ID NO: 1 shown below are non-limiting exemplary residues that can be mutated (e.g., by substitution, deletion or insertion) to generate variants with one or more desirable properties.

(SEQ ID NO: 1)
MTSETGPSTGDATLRRRIQPWEFEVFFDPRELRKETCLLYEIKWGRSRKIWRNSGQNTSKHVEVNFIEKL
ISERHFCPSVSCSITWELSWSPCWECSKAIREFLSQHPNITLVIYVARLFHHMDQHNRQGLRDLINSGVT
IQIMRAEEYDHCWRNFVNYSPGEEAHWPRYPPLWMTMYALELHCIILSLPPCLKITRRCQHQHTFFSLNF
QNCHYQMIPPQTLYAAGIIQPSMTWR
(SEQ ID NO: 19)
ACAAGCGAAACCGGCCCTAGCACCGGCGACGCCACCCTGCGCAGACGGATTCAGCCCTGGGAGTTCGAGG
TGTTCTTCGACCCTAGAGAGCTGAGAAAGGAAACCTGCCTGCTGTACGAGATCAAGTGGGGCAGAAGCAG
AAAGATCTGGAGAAACAGCGGGCAGAACACAAGCAAGCACGTGGAGGTGAACTTCATCGAGAAGCTGATC
AGCGAGAGACACTTCTGCCCTAGCGTGTCTTGCAGCATCACCTGGTTCCTGAGCTGGAGCCCCTGCTGGG
AGTGCAGCAAGGCCATCAGAGAGTTCCTGTCTCAGCACCCCAACATCACCCTGGTGATCTACGTGGCTAG
ACTGTTCCACCACATGGATCAGCACAACAGACAAGGCCTGCGGGACCTGATCAACAGCGGCGTGACCATT
CAGATCATGAGAGCCGAGGAGTACGACCACTGCTGGAGAAACTTCGTGAACTACAGCCCCGGCGAGGAGG
CCCACTGGCCTAGATACCCCCCCCTGTGGATGACCATGTACGCCCTGGAGCTGCACTGCATCATCCTGAG
CCTGCCCCCCTGCCTGAAGATCACAAGAAGATGTCAGCATCAGCACACCTTCTTCAGCCTGAACTTTCAG
AACTGCCACTATCAGATGATCCCCCCTCAGACCCTGTACGCCGCCGGCATCATTCAGCCTAGCATGACCT
GGAGA

The deaminase can be, for example, a deaminase (SEQ ID NO: 7) from Mongolian gerbil (Meriones unguiculatus) encoded by a nucleic acid sequence of SEQ ID NO: 25. The residues in bold and underlined in SEQ ID NO: 7 shown below are non-limiting exemplary residues that can be mutated (e.g., by substitution, deletion or insertion) to generate variants with one or more desirable properties.

(SEQ ID NO: 7)
MSSETGPAADPTLRRRIEPQEFGAFFDPQLLRKETCLLYEINWGGRHSVWRHTGQNTDRHAEINFIEKFT
SERYFCPFTRCSITWFLSWSPCGECCRAIVEFLSRYPNVTLFIYVARLYHHTDERNRQGLRDLCRRGVTI
RIMTEQECYYCWRNFVNYSPSNEAHWPRYPHLWVRMYVLELYCILLGLPPCLKILRRNONQLTIFNLAFQ
HCHFQRLPYYIF
(SEQ ID NO: 25)
AGCAGCGAAACCGGCCCCGCCGCCGACCCCACCCTGAGAAGAAGAATCGAGCCCCAAGAGTTCGGCGCCT
TCTTCGACCCTCAGCTGCTGAGAAAGGAAACCTGCCTGCTGTACGAGATCAACTGGGGCGGCAGACACAG
CGTGTGGAGACACACCGGGCAGAACACCGACAGACACGCCGAGATCAACTTCATCGAGAAGTTCACAAGC
GAGAGATACTTCTGCCCCTTCACAAGATGCAGCATCACCTGGTTCCTGAGCTGGAGCCCCTGCGGCGAGT
GTTGCAGAGCCATCGTGGAGTTCCTGAGCAGATACCCCAACGTGACCCTGTTCATCTACGTGGCTAGACT
GTACCACCACACCGACGAGAGAAACAGACAAGGCCTGCGGGACCTGTGCAGAAGAGGCGTGACCATCAGA
ATCATGACCGAGCAAGAGTGCTACTACTGCTGGAGAAACTTCGTGAACTACAGCCCTAGCAACGAGGCCC
ACTGGCCTAGATACCCCCACCTGTGGGTGAGAATGTACGTGCTGGAGCTGTACTGCATCCTGCTGGGCCT
GCCCCCCTGCCTGAAGATCCTGAGAAGAAATCAGAATCAGCTGACCATCTTCAACCTGGCCTTTCAGCAC
TGCCACTTTCAGAGACTGCCCTACTACATCTTC

The deaminase can be, for example, a deaminase (SEQ ID NO: 13) from Little brown bat (Myotis lucifugus) encoded by a nucleic acid sequence of SEQ ID NO: 31. The residues in bold and underlined in SEQ ID NO: 13 shown below are non-limiting exemplary residues that can be mutated (e.g., by substitution, deletion or insertion) to generate variants with one or more desirable properties.

(SEQ ID NO: 13)
MASDAGSSAGDPTLRRRIEPWDFEAIFDPRELRKEACLLYEIKWGPCHKIWRHSGKNTTRHVEVNFIEKI
TSERQFCSSTSCSIIWFLSWSPCWECSKAITEFLRQRPGVTLVIYVARLYHHMDEQNRQGLRDLIKSGVT
IQIMTTPEYDYCWRNFVNYPPGKDTHCPMYPPLWMKLYALELHCIILSLPPCLMISRRCQKQLTWYRLNL
QNCHYQQIPPHILLATAWI
(SEQ ID NO: 31)
GCAAGCGACGCCGGCAGCAGCGCCGGCGACCCCACCCTGAGAAGAAGAATCGAGCCCTGGGACTTCGAGG
CCATCTTCGACCCTAGAGAGCTGAGAAAGGAGGCCTGCCTGCTGTACGAGATCAAGTGGGGCCCCTGCCA
CAAGATCTGGAGACACAGCGGCAAGAACACCACAAGACACGTGGAGGTGAACTTCATCGAGAAGATCACA
AGCGAGAGACAGTTTTGCAGCAGCACATCTTGCAGCATCATCTGGTTCCTGAGCTGGAGCCCCTGCTGGG
AGTGCAGCAAGGCCATCACCGAGTTCCTGAGACAAAGACCCGGCGTGACCCTGGTGATCTACGTGGCTAG
ACTGTACCACCACATGGACGAGCAGAACAGACAAGGCCTGCGGGACCTGATCAAGAGCGGCGTGACCATT
CAGATCATGACCACCCCCGAGTACGACTACTGCTGGAGAAACTTCGTGAACTACCCCCCCGGCAAGGACA
CCCACTGCCCCATGTACCCCCCCCTGTGGATGAAGCTGTACGCCCTGGAGCTGCACTGCATCATCCTGAG
CCTGCCCCCCTGCCTGATGATCAGCAGAAGATGTCAGAAGCAGCTGACCTGGTACAGACTGAACTTGCAG
AACTGCCACTATCAGCAGATCCCCCCCCACATCCTGCTGGCCACCGCCTGGATC

In some embodiments, the deaminase or deaminase domain comprises an amino acid sequence that is about, at least, or at least about 60%, 65%, 70%, 75%, 80%, 85%, 90%, 95%, 96%, 97%, 98%, 99% or 99.5% identical to any one of amino acid sequences set forth in SEQ ID NOs: 1-18. In some embodiments, the deaminase or deaminase domain comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations compared to any one of the amino acid sequences set forth in SEQ ID NOs: 1-18. In some embodiments, the deaminase or deaminase domain comprises an amino acid sequence having one, two, three, four, five, six, seven, eight, nine, ten, eleven, twelve, thirteen, fourteen, fifteen, sixteen, seventeen, eighteen, nineteen, or twenty mismatches relative to any one of SEQ ID NOs: 1-18. In some embodiments, the deaminase or deaminase domain comprises an amino acid sequence of any one of SEQ ID NOs: 1-18.

In some embodiments, the deaminase or deaminase domain described herein comprises one or more mutations as compared to a parent deaminase or deaminase domain (e.g., a wild type deaminase). The deaminase or deaminase domain can comprise one or more mutations with respect to any sequence of SEQ ID NOs: 1, 7, and 13. For example, the deaminase or deaminase domain can comprise one or more mutations of R33A, K34A, W90Y, H126E and H122A with respect to wildtype deaminase (SEQ ID NO: 1). For example, SEQ ID NO: 2 is the R33A variant of SEQ ID NO: 1, SEQ ID NO: 3 is the K34A variant of SEQ ID NO: 1, SEQ ID NO: 4 is the W90Y variant of SEQ ID NO: 1, SEQ ID NO: 5 is the W90Y/H126E variant of SEQ ID NO: 1, and SEQ ID NO: 6 is the H122A variant of SEQ ID NO: 1. The deaminase or deaminase domain can comprise one or more mutations of R32A, K33A, W89Y, R125E and H121A with respect to wildtype deaminase (SEQ ID NO: 7). For example, SEQ ID NO: 8 is the R32A variant of SEQ ID NO: 7, SEQ ID NO: 9 is the K33A variant of SEQ ID NO: 7, SEQ ID NO: 10 is the W89Y variant of SEQ ID NO: 7, SEQ ID NO: 11 is the W89Y/R125E variant of SEQ ID NO: 7, and SEQ ID NO: 12 is the H121A variant of SEQ ID NO: 7. The deaminase or deaminase domain can comprise one or more mutations of R33A, K34A, W90Y, Q126E and H122A with respect to wildtype deaminase (SEQ ID NO: 13). For example, SEQ ID NO: 14 is the R33A variant of SEQ ID NO: 13, SEQ ID NO: 15 is the K34A variant of SEQ ID NO: 13, SEQ ID NO: 16 is the W90Y variant of SEQ ID NO: 13, SEQ ID NO: 17 is the W90Y/Q126E variant of SEQ ID NO: 13, and SEQ ID NO: 18 is the H122A variant of SEQ ID NO: 13.

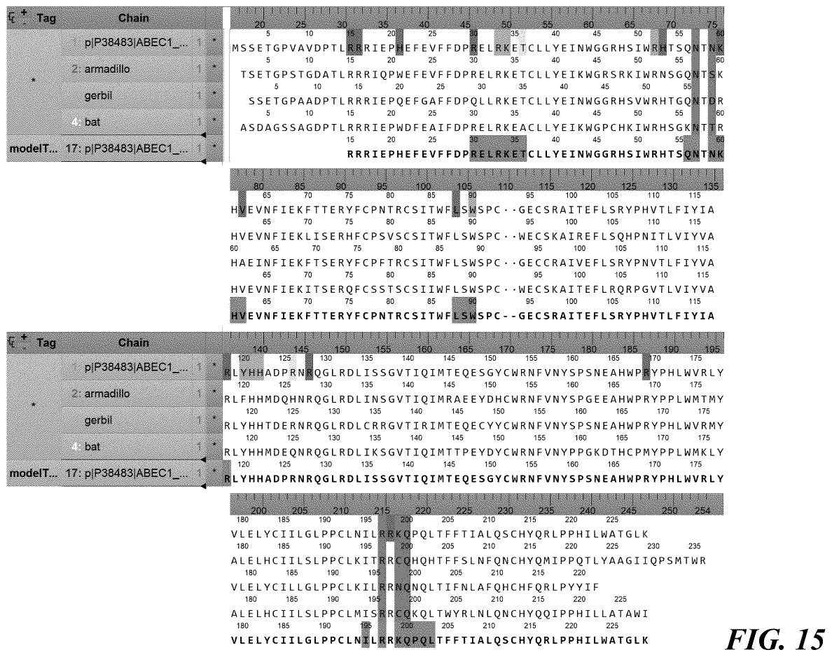

One or more amino acid residues of any one of the deaminases disclosed herein can be mutated (e.g., substituted, deleted, or with insertion) to generate variants of the deaminases. For example, one or more of the residues highlighted in the sequence alignment of FIG. 15 can be mutated to generate variants. These non-limiting exemplary residues include any one of the amino acid residues of RELRKET (positions 30-36), R at position 52, QN (positions 56 and 57), NKHV (positions 59-62), LSW (positions 88-90), R at position 118, YHH at positions 120-122, R at position 126, R at position 128, R at position 169, I at position 195, R at position 197, R at position 198, and KQPQL at positions 199-203) of rAPOBEC1 or of armadillo deaminase (SEQ ID NO: 1); any combination thereof, or functional equivalent(s) thereof. The deaminases or deaminase domains disclosed herein can comprise, for example, amino acid mutation(s) (e.g., substitution(s), deletion(s), or insertion(s)) at one or more positions functionally equivalent to F23, P29, R30, E31, L32, R33, K34, E35, T36, C37, L38, L39, R52, S55, Q56, N57, N59, K60, H61, V62, N65, F66, I67, E68, K69, Y75, N79, L88, S89, W90, S91, R106, Y107, P108, H109, T111, I114, R118, Y120, H121, H122, R126, R128, S137, G138, V139, T140, R169, I195, R197, R198, K199, Q200, P201, Q202, and L203 in the deaminase of SEQ ID NO: 1. In some embodiments, the deaminases or deaminase domains disclosed herein can one or more amino acid substitutions of R33A, K34A, W90Y, H126E and H122A. The amino acid substitution can be, for example, a mutation to nonpolar amino acid, a polar amino acid, a positively charged amino acid, a negatively charged amino acid, a hydrophobic amino acid, an aromatic amino acid, an aliphatic amino acid, a small amino acid, and/or a hydrophilic amino acid.

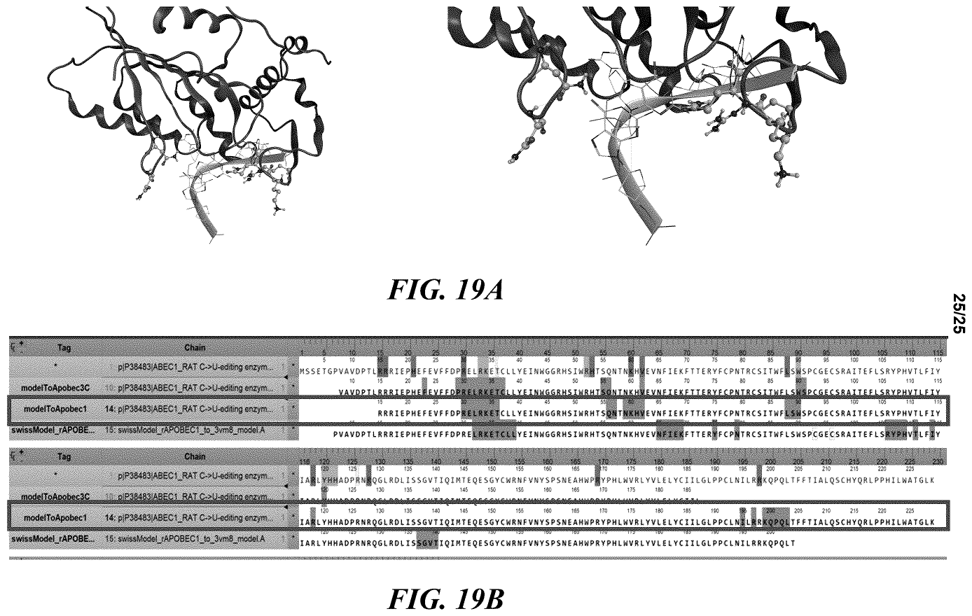

As understood by those skilled in the art, identification of potential amino acid residues for mutation in a deaminase can be accomplished using molecular modeling such as homology modeling or ab initio modeling to construct a three-dimensional structure of the deaminase. Amino acid residues in a deaminase involved in binding with ssDNA or in close proximity to the ssDNA binding site can be identified as potential sites for mutations. The term “homology model” refers to an structural model derived from known three-dimensional structure(s). Known three-dimensional structures can be any deaminase structure (e.g., an APOBEC3 or APOBEC1 family deaminase) derived from experimental data such as crystallographic and NMR structure determination. Generation of the homology model, termed “homology modeling”, can include sequence alignment, structural alignment, residue replacement, residue conformation adjustment through energy minimization, or a combination thereof. FIG. 19A depicts a schematic representation of a deaminase homology model constructed from homology modeling. Three-dimensional structures can also be obtained from ab initio modeling using empirical or semi-empirical techniques. Molecular modeling can be performed using, for example, computer programs such as SWISS-MODEL, MOE, Rosetta, Modeller and others commercial or publicly available computer software identifiably by those skilled in the art. FIG. 19B shows an alignment of potential sites for mutations identified by different molecular modeling programs.

In some embodiments, the deaminase or deaminase domain is encoded by a nucleic acid sequence that is about, at least, or at least about 60%, 65%, 70%, 75%, 80%, 85%, 90%, 95%, 96%, 97%, 98%, 99% or 99.5% identical to any one of amino acid sequences set forth in SEQ ID NOs: 19-36. In some embodiments, the deaminase or deaminase domain is encoded by a nucleic acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations compared to any one of the nucleic acid sequences set forth in SEQ ID NOs: 19-36. In some embodiments, the deaminase or deaminase domain is encoded by a nucleic acid sequence having one, two, three, four, five, six, seven, eight, nine, ten, eleven, twelve, thirteen, fourteen, fifteen, sixteen, seventeen, eighteen, nineteen, or twenty mismatches relative to any one of SEQ ID NOs: 19-36. In some embodiments, the deaminase or deaminase domain is encoded by a nucleic acid sequence of any one of SEQ ID NOs: 19-36.

### Cas9 Domain

The fusion proteins described herein also comprise a Cas9 domain, when in conjunction with a bound guide RNA (gRNA), capable of specifically binding to a target nucleic acid sequence. The term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. In some embodiments, a Cas9 refers to Cas9 from Corynebacterium ulcerans, Corynebacterium diphtheria, Spiroplasma syrphidicola, Prevotella intermedia, Spiroplasma taiwanense, Streptococcus iniae, Belliella baltica, Psychroflexus torquisI, Streptococcus thermophilus, Listeria innocua, Campylobacter jejuni, Neisseria. Meningitidis, Streptococcus pyogenes, Staphylococcus aureus, or S. schleiferi.

The Cas9 domain of a fusion protein can comprise a full-length amino acid of a Cas9 protein or a fragment thereof. For example, the Cas9 domain can comprise a truncated version of a nuclease domain or no nuclease domain at all.

The Cas9 domain can be a nuclease active Cas9 domain, a nuclease inactive Cas9 domain, a dead Cas9, a Cas9 fragment, or a Cas9 nickase. In some embodiments, the Cas9 domain is a nuclease active domain. For example, the Cas9 domain may be a Cas9 domain that cuts both strands of a duplexed nucleic acid (e.g., both strands of a duplexed DNA molecule).

In some embodiments, the Cas9 domain is a nuclease-inactive Cas9 domain or dead Cas9 (dCas9). For example, the dCas9 domain can bind to a duplexed nucleic acid molecule (e.g., via a gRNA molecule) without cleaving either strand of the duplexed nucleic acid molecule. Additional suitable Cas9 domains will be apparent to those of skill in the art based on this disclosure and knowledge in the field, and are within the scope of this disclosure.

Methods for generating a Cas9 protein (or a fragment thereof) having an inactive DNA cleavage domain are known in the art (e.g., Jinek et al., Science. 337:816-821 (2012); Qi et al., “Repurposing CRISPR as an RNA-Guided Platform for Sequence-Specific Control of Gene Expression” (2013) Cell. 28; 152 (5): 1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H841A completely inactivate the nuclease activity of Streptococcus pyogenes Cas9 (Jinek et al., Science. 337:816-821 (2012); Qi et al., Cell. 28; 152 (5): 1173-83 (2013). Additional exemplary suitable nuclease-inactive Cas9 domains include, but are not limited to, D10A, D10A/D839A/H840A, and D10A/D839A/H840A/N863A mutant domains with respect to wild type Cas9 (e.g., SpCas9) (e.g., Mali et al., CAS9 transcriptional activators for target specificity screening and paired nickases for cooperative genome engineering. Nature Biotechnology. 2013; 31 (9): 833-838, the entire contents of which are incorporated herein by reference). Mutations can be introduced to any suitable Cas9 known in the art, including but not limited to Cas9 from Corynebacterium ulcerans, Corynebacterium diphtheria, Spiroplasma syrphidicola, Prevotella intermedia, Spiroplasma taiwanense, Streptococcus iniae, Belliella baltica, Psychroflexus torquisI, Streptococcus thermophilus, Listeria innocua, Campylobacter jejuni, Neisseria. Meningitidis, Streptococcus pyogenes, Staphylococcus aureus, or S. schleiferi. Additional suitable nuclease-inactive Cas9 domains will be apparent to those of skill in the art based on this disclosure.

In some embodiments, the Cas9 domain is a Cas9 nickase. The Cas9 nickase can be a Cas9 protein that is capable of cleaving only one strand of a duplexed nucleic acid molecule (e.g., a duplexed DNA molecule). In some embodiments, a Cas9 nickase can have an active HNH nuclease domain and is able to cleave the non-targeted strand of DNA, i.e., the strand bound by the gRNA. The Cas9 nickase can have an inactive RuvC nuclease domain and is not able to cleave the targeted strand of the RNA, i.e., the strand where base editing is desired.

In some embodiments, the Cas9 domain is a variant sharing homology to Cas9 or a fragment thereof. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to wild type Cas9. In some embodiments, the Cas9 domain comprises one or more point mutations with respect to the corresponding wild type Cas9. Mutations may comprise substituting one or more charged and/or polar residues (e.g., aspartic acid, histidine, asparagine) to alanine.

In some embodiments, the Cas9 domains described herein can have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editing fusion proteins provided herein may need to be placed at a precise location, for example where a target base is placed within a 4 base region (e.g., a “deamination window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the fusion proteins provided herein can contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

### Uracil Glycosylase Inhibitor (UGI)

In some embodiments, the fusion protein comprises one or more uracil glycosylase inhibitor (UGI) domain. The term “uracil glycosylase inhibitor” or “UGI,” as used herein, refers to a protein that is capable of inhibiting a uracil-DNA glycosylase base-excision repair enzyme. Without wishing to be bound by any particular theory, cellular DNA-repair response to the presence of U:G heteroduplex DNA may be responsible for the decrease in nucleobase editing efficiency in cells. For example, uracil DNA glycosylase (UDG) catalyzes removal of U from DNA in cells, which may initiate base excision repair, with reversion of the U:G pair to a C:G pair as the most common outcome. In some embodiments, fusion proteins comprising a UGI domain can inhibit human UDG activity, enabling increased deamination efficiency. A UGI domain can comprise a wild-type UGI or a UGI variant including a fragment of UGI or a protein homologous to a UGI or UGI fragment.

In some embodiments, the present disclosure includes a fusion protein comprising a deaminase and Cas9 nickase domain further fused to at least one UGI domain. The UGI domain can be fused to the Cas9 domain and/or the deaminase domain either directly or via an optional linker. In some embodiments, the fusion protein comprises two UGI domains. The one or more UGI domains, the deaminase domain, and the Cas9 domain can be connected to one another in any suitable configuration. For example, in some embodiments, the fusion protein comprises the structure of [deaminase domain]-[Cas9 domain]-[UGI]-[UGI] connected either directly or via an optional linker.

A UGI domain can be any protein or fragment thereof capable of inhibiting (e.g., sterically blocking) a uracil-DNA glycosylase base-excision repair enzyme. Additionally, any proteins that block or inhibit base-excision repair are also within the scope of this disclosure. In some embodiments, a UGI is a protein that binds uracil. In some embodiments, a UGI is a protein that binds uracil in DNA. In some embodiments, a UGI is a catalytically inactive uracil DNA-glycosylase protein. In some embodiments, a UGI is a catalytically inactive uracil DNA-glycosylase protein that does not excise uracil from the DNA. Suitable UGI protein and nucleotide sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264:1163-1171 (1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419 (1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887 (1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346 (1999), the entire contents of which are incorporated herein by reference.

In some embodiments, the fusion proteins comprising a UGI further comprise a nuclear targeting sequence, for example a nuclear localization sequence. In some embodiments, fusion proteins provided herein further comprise a nuclear localization sequence (NLS). The NLS can be fused to the N-terminus or C-terminus of the fusion protein. In some embodiments, the NLS can be fused to the N-terminus or C-terminus of the UGI, the N-terminus or C-terminus of the Cas9 domain, or the N-terminus or C-terminus of the deaminase domain. The fusion can be either direct or via a linker.

The Cas9 domain, the deaminase domain, and optionally the UGI and NLS can be fused to one another via a linker. The term “linker,” as used herein, refers to a chemical group or a molecule linking two molecules or moieties. In some embodiments, a linker joins a Cas9 domain (e.g., a Cas9 nickase) and a deaminase domain. In some embodiments, a linker joins a Cas9 domain with a UGI domain. In some embodiments, a linker join the deaminase domain with a UGI domain. In some embodiments, a linker joins one UGI domain with another UGI domain. A linker can be an amino acid, a peptide or protein, an organic molecule, a polymer or chemical moiety. The sequence, length and flexibility of the linker can vary in different embodiments. In some embodiments, the linker can have about 3-100 amino acids in length. Suitable linker motifs and configurations are described in published literatures (see, e.g., Chen et al., Fusion protein linkers: property, design and functionality. Adv Drug Deliv Rev. 2013; 65 (10): 1357-69 and Guilinger J P, Thompson D B, Liu D R. Fusion of catalytically inactive Cas9 to FokI nuclease improves the specificity of genome modification. Nat. Biotechnol. 2014; 32 (6): 577-82, the contents of which are incorporated herein by reference) and will be apparent to those of skill in the art. In some embodiments, the linker comprises a (GGS)ₙ, (G)ₙ, ((GGGGS)ₙ motif, wherein n is 1, 2, 3, 4, 5, 6, 7, 8, 9, 19, 11, 12, 13, 14, 15, 16, 17, 18, 19, or 20. In some embodiments, the optional linker comprises a (GGS) n motif, wherein n is 1, 3, or 7.

The deaminase domain, the Cas9 domain and optionally one or more UGI and NSL can be connected to one another in a variety of suitable configurations. In some embodiments, the deaminase domain is fused to the N-terminus of the Cas9 domain. In some embodiments, the deaminase domain is fused to the C-terminus of the Cas9 domain. In some embodiments, a base editing fusion protein described herein comprises the structure from the N-terminus to the C-terminus: [deaminase domain]-[Cas9 domain], in which the deaminase domain and the Cas9 domain are connected directly or via a linker. In some embodiments, a base editing fusion protein described herein comprises a structure from the N-terminus to the C-terminus selected from the following: [deaminase domain]-[Cas9 domain]-[UGI], [UGI]-[deaminase domain]-[Cas9 domain] or [deaminase domain]-[UGI]-[Cas9 domain]. In some embodiments, more than one UGI domain can be fused to the deaminase and/or Cas9 domain. For example, a base editing fusion protein described herein can have a structure of [deaminase domain]-[Cas9 domain]-[UGI]-[UGI]. In some embodiments, the deaminase domain is a mammalian deaminase (e.g., any one of SEQ ID NOs: 1-18 or a sequence having at least 85%, 90%, 95%, 96%, 97%, 98%, 99% or greater identify to any one of SEQ ID NOs: 1-18) or a fragment thereof and the Cas9 domain is a Cas9 nickase or a fragment thereof.

The base editing fusion protein used herein can be a portion or variant of BE1 base editor, BE2 base editor, BE3 base editor, HF-BE3, BE4, BE4-GAM, YE1-BE3, EE-BE3, YE2-BE3, YEE-BE3, VQR-BE3, VRER-BE3, VRER-BE3, Sa-BE3, Sa-BE4, SaBE4-Gam, SaKKH-BE3, Cas12a-BE, xBE3 described in Rees and Liu, Nat Rev Genet. 2018 December; 19 (12): 770-788, the contents of which are incorporated herein by reference.

The base editing fusion protein described herein can efficiently generate an intended mutation, such as a point mutation from C to T, in a nucleic acid without generating an unintended mutation such as an unintended point mutation. In some embodiments, the base editing fusion protein described herein can modify a specific nucleotide base without generating undesired byproducts including indels, translocations and other DNA rearrangements. The term “indel”, as used herein, refers to the insertion or deletion of one or more nucleotide base within a nucleic acid. In some embodiments, the fusion protein described herein can generate fewer than 10%, 9%, 8%, 7%, 6%, 5%, 4%, 3%, 2%, 1%, 0.5%, 0.2%, 0.1%, 0.01%, 0.001%, or less indels. In some embodiments, the fusion protein described herein can generate a ratio of intended point mutations to indels that is at least 5:1, 10:1, 20:1, 30:1, 40:1, 50:1, 100:1, 200:1, 400:1, 600:1, 800:1, 1000:1 or higher. In some embodiments, the fusion protein described herein can generate fewer than 10%, 9%, 8%, 7%, 6%, 5%, 4%, 3%, 2%, 1%, 0.5%, 0.2%, 0.1%, 0.01%, 0.001%, or less translocations or other DNA rearrangement. In some embodiments, the fusion protein described herein does not generate any indel, DNA translocation or other DNA rearrangement.

Guide RNAs (gRNAs)

The base editing fusion protein described herein can, for example, be in complex with or associated with a guide RNA (gRNA). Accordingly, disclosed herein also includes a fusion protein and a guide RNA bound to the Cas9 domain of the fusion protein. Disclosed herein can also comprises a composition comprising a fusion protein described herein or a nucleic acid sequence encoding the fusion protein and one or more guide RNAs targeting one or more target genes.

In some embodiments, the gRNA comprise 5′ to 3′: a crRNA and a tracrRNA, wherein the crRNA and tracrRNA hybridize to form a duplex. In some embodiments, the crRNA comprises a spacer sequence capable of targeting a target sequence in a target nucleic acid (e.g., genomic DNA molecule) and a crRNA repeat sequence. In some embodiments, the tracrRNA comprises a tracrRNA anti-repeat sequence and a 3′ tracrRNA sequence. In some embodiments, the 3′ end of the crRNA repeat sequence is linked to the 5′ end of the tracrRNA anti-repeat sequence, e.g., by a tetraloop, wherein the crRNA repeat sequence and the tracrRNA anti-repeat sequence hybridize to form the sgRNA. In some embodiments, the sgRNA comprises 5′ to 3′: a spacer sequence, a crRNA repeat sequence, a tetraloop, a tracrRNA anti-repeat sequence, and a 3′ tracrRNA sequence. In some embodiments, the sgRNA comprise a 5′ spacer extension sequence. In some embodiments, the sgRNA comprise a 3′ tracrRNA extension sequence. The 3′ tracrRNA can comprise, or consist of, one or more stem loops, for example one, two, three, or more stem loops.

In some embodiments, the guide RNA is from 15-100 nucleotides long and comprises a sequence of at least 10 contiguous nucleotides that is complementary to a target sequence. In some embodiments, the guide RNA is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA is a single-guide RNA (sgRNA).

The guide RNA disclosed herein can target any sequence of interest via the spacer sequence in the crRNA. A spacer sequence in a gRNA is a sequence (e.g., a 20 nucleotide sequence) that defines the target sequence (e.g., a DNA target sequences, such as a genomic target sequence) of a target gene of interest. In some embodiments, the spacer sequence range from 15 to 30 nucleotides. For example, the spacer sequence can be, can be about, can be at least, or can be at most 10, 15, 16, 17, 18, 19, 29, 21, 22, 23, 24, 25, 26, 27, 28, 29, 30, 40, 50, or a number or a range between any of these values, of nucleotides in length. In some embodiments, a spacer sequence contains 20 nucleotides.

The “target sequence” is in a target gene that is adjacent to a PAM sequence and is the sequence to be modified by an RNA-guided nuclease (e.g., Cas9 nickase). The “target sequence” is on the so-called PAM-strand in a “target nucleic acid,” which is a double-stranded molecule containing the PAM-strand and a complementary non-PAM strand. One of skill in the art recognizes that the gRNA spacer sequence can hybridize to the complementary sequence located in the non-PAM strand of the target nucleic acid of interest. Thus, the gRNA spacer sequence is the RNA equivalent of the target sequence. The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In the base editing described herein, the spacer sequence is designed to hybridize to a region of the target nucleic acid that is located 5′ of a PAM recognizable by a Cas9 domain used in the system. The spacer can perfectly match the target sequence or can have mismatches. Each Cas9 domain has a particular PAM sequence that it recognizes in a target DNA. For example, S. pyogenes recognizes in a target nucleic acid a PAM that comprises the sequence 5′-NRG-3′, where R comprises either A or G, where Nis any nucleotide and N is immediately 3′ of the target nucleic acid sequence targeted by the spacer sequence.

In some embodiments, the target sequence is a DNA sequence. In some embodiments, the target sequence is a sequence in the genome of a mammal. In some embodiments, the target sequence is a sequence in the genome of a human. In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the 3′ end of the target sequence is immediately adjacent to a canonical PAM sequence (NGG). For example, in a sequence comprising 5′-NNNNNNNNNNNNNNNNNNNNNRG-3′ (SEQ ID NO: 55), the target nucleic acid can be the sequence that corresponds to the Ns, wherein N can be any nucleotide, and the underlined NRG sequence (R is G or A) is the S. pyogenes PAM. In some embodiments, the 3′ end of the target sequence is not immediately adjacent to a canonical PAM sequence. In some embodiments, the PAM sequence used in the compositions and methods of the present disclosure as a sequence recognized by SpCas9 is NGG.

In some embodiments, the percent complementarity between the spacer sequence and the target nucleic acid is about, at least, at least about, at most or at most about 30%, 40%, 50%, 60%, 65%, 70%, 75%, 80%, 85%, 90%, 95%, 97%, 98%, 99%, or 100%. In some embodiments, the spacer sequence of the guide RNA and the target nucleic acid in the target gene is 100% complementary. In some embodiments, the percent complementarity between the spacer sequence and the target nucleic acid is 100% over the six contiguous 5′-most nucleotides of the target sequence of the complementary strand of the target nucleic acid. In some embodiments, the percent complementarity between the spacer sequence and the target nucleic acid is at least 60% over about 20 contiguous nucleotides. In other embodiments, the spacer sequence of the guide RNA and the target sequence in the target gene can contain up to 10 mismatches, e.g., up to 9, up to 8, up to 7, up to 6, up to 5, up to 4, up to 3, up to 2, or up to 1 mismatch.

The guide RNA can be complementary to a sequence associated with a disease or disorder. In some embodiments, the guide RNA is complementary to a sequence associated with a disease or disorder having a mutation in a gene.

In some embodiments, the gRNA is a chemically modified gRNA. Various types of RNA modifications can be introduced to the gRNAs to enhance stability, reduce the likelihood or degree of innate immune response, and/or enhance other attributes as described in the art. The gRNAs described herein can comprise one or more modifications including internucleoside linkages, purine or pyrimidine bases, or sugar. In some embodiments, a modification is introduced at the terminal of a gRNA with chemical synthesis or with a polymerase enzyme. Examples of modified nucleic acids and their synthesis are disclosed in WO2013/052523. Synthesis of modified polynucleotides is also described in Verma and Eckstein, Annual Review of Biochemistry, vol. 76, 99-134 (1998).

The chemically-modified gRNA can comprise phosphorothioated 2′-O-methyl nucleotides at the 3′ end and the 5′ end of the gRNA. In some embodiments, the chemically-modified gRNA comprises phosphorothioated 2′-O-methyl nucleotides at the 3′ end of the gRNA. In some embodiments, the chemically-modified gRNA comprises phosphorothioated 2′-O-methyl nucleotides at the 5′end of the gRNA. In some embodiments, the chemically-modified gRNA comprises three or four phosphorothioated 2′-O-methyl nucleotides at the 3′ end and/or three or four at the 5′ end of the gRNA.

In some embodiments, more than one guide RNA can be used with a fusion protein described herein. For example, two, three, four, five, six or more guide RNA can be used with a fusion protein described herein. Each guide RNA can contain a different targeting sequence, such that the deaminase cleaves more than one target nucleic acid, thus generating more than one gene edits to the target nucleic acid sequences.

In some embodiments, the gRNAs described herein can be produced in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in WO2013/151666. Polynucleotides constructs and vectors can be used to in vitro transcribe a gRNA described herein.

### Methods for Editing Nucleic Acids

Provided herein also includes methods of using the base editing fusion proteins, complexes and compositions described herein for editing nucleic acids. In some embodiments, a method comprises contacting a DNA molecule with a fusion protein of herein described and a guide RNA (gRNA) targeting a target nucleotide sequence of the DNA molecule, thereby editing the DNA molecule by deaminating a nucleotide base within the target nucleotide sequence of the DNA molecule. The DNA molecule can be in contact with the fusion protein and gRNA in an effective amount and under conditions suitable for the deamination of a nucleotide base. In some embodiments, a method comprises contacting a composition comprising a fusion protein described herein or a nucleic acid sequence encoding the fusion protein and one or more guide RNAs targeting one or more target genes in an effective amount and under conditions suitable for the deamination of a nucleotide base.

In some embodiments, the contacting is performed in vivo in a subject susceptible to having, having, or diagnosed with the disease or disorder. In some embodiments, the disease or disorder is a disease associated with a point mutation, or a single-base mutation, in the genome. In some embodiments, the disease is a genetic disease, a cancer, a metabolic disease, or a lysosomal storage disease.

In some embodiments, the target DNA sequence and/or the target gene comprises a sequence associated with a disease or disorder and wherein the deamination of a nucleotide base results in a sequence that is not associated with a disease or disorder. In some embodiments, the target DNA sequence comprises a T to C point mutation associated with a disease or disorder and wherein the deamination of the mutant C base results in a sequence that is not associated with a disease or disorder.

In some embodiments, the deamination of the target nucleobase results in the correction of a genetic defect, e.g., in the correction of a point mutation that leads to a loss of function in a gene product. The genetic defect can be associated with a disease or disorder. In some embodiments, the methods provided herein are used to introduce a deactivating point mutation into a gene or allele that encodes a gene product that is associated with a disease or disorder (e.g., an oncogene). In some embodiments, the sequence associated with a disease or disorder is a gene encoding a protein. The deamination results in a disrupted or deactivated gene such that expression of a functional protein from the gene is reduced or inhibited. For example the deamination can generate a premature stop codon in a coding sequence, which results in the expression of a truncated gene product, e.g., a truncated protein lacking the function of the full-length protein.

In some embodiments, the methods described herein can restore the function of dysfunctional gene via base editing. Generally, any single nucleotide polymorphisms (SNPs) involving a T to C point mutation while having a nearby Cas recognizable PAM sequence can be corrected using the method herein described. Deamination of the mutant C back to U corrects the mutation. Examples of genes comprising a T to C point mutation associated with a disease or disorder include, but are not limited to, CBS, DPYS, AGA, ALDOB, RFX6, TMEM67, ERCC6, GJC2, PC, ADSL, TPP1, BEST1, ACAT1, SMPD1, LDLR, TLL1, SLC26A2, GBA, EIF2B3, GAN, ANKH, FBLN5, ROBO2, KLF6, DDX11, FOXF1, NOTCH3, MMP13, ITGB2, ABCA1, MT-TL2, MT-TI, MT-ND1, PRPS1, F8, F9, TAZ, BTK, FOXP3, CASK, MECP2, MVK, TEAD1, SOS1, FGFR2, GP9, GPI, PTH, KIT, MAPT, LMNA, KRT5, HBG2, GH1, GRN, GPD2, NR3C1, SMC1A, SGSH, MRPS22, CLN6, MFN2, RHBDF2, UVSSA, POC1A, HINT1, COL4A5, SMN1, HCFC1, MVK, SLC29A3, DLD, BRAF, RYR1, MYBPC3, MYH7, USH2A, BRCA2, BRCA1, KARS, KAT6A, KCNQ1, SCN5A, SCN1A, SCN10A, MSH2, GBA, DYSF, ATP6V50A2, FOLR1, STX11, IFT140, OTC, PRPH2, ITGB2, SAMHD1, ACTB, RRM2B, PGM3, HNRNPA1, DNMT3A, CDKN1C, MTCO3, GATA1, DNAL4, SCN11A, TRNT1, DCX, LMNA, MYBPC3, MTHFR, PTCH1, AGXT, SGCA, ACTN2, SCN1A, LAMA2, EDA, COL6A1, KCNH2, SCN2A, CLCN1, NDUFS2, and TP53. Detailed information about the above gene symbols, including genotypes, sequences, and associated genetic diseases, can be found in public domains such as NCBI ClinVar database as will be understood to a person skilled in the art.

In some embodiments, the target gene sequence does not comprise a T to C point mutation. The deaminase can introduce a deactivating mutation or a premature stop codon in a coding sequence in the target gene sequence, resulting in the expression of a non-functional and/or truncated gene product. In some embodiments, the target sequence comprises a LPA gene or a variant thereof. In some embodiments, the target sequence comprises an ANGPTL3 gene or a variant thereof.

In some embodiments, the guide RNA used herein can comprise two or more guide RNAs targeting two or more target genes and the methods provided here can edit two more target genes. In some embodiments, editing of the target genes results a significant reduction in the gene products of the two or more target genes (e.g., mRNA or protein level). In some embodiments, the gene products of the two or mor target genes are reduced by at least 40%, 45%, 50%, 55%, 60%, 65%, 70%, 75%, 80%, 85%, 90%, 95%, 98%, 99%, 100% or a number or a range between any two of these values.

### Methods for Analyzing Off-Target Deamination

Provided herein also includes methods of using the base editing fusion proteins and guide RNAs described herein to evaluate the ability of base editing proteins to deaminate cytosines in DNA regions unrelated to their on-target loci (i.e., spurious off-target deamination activity). In some embodiments, a method can comprise transfecting a cell with a first Cas9 domain or a nucleic acid encoding the first Cas9 domain to produce a cell expressing the first Cas9 domain and transfecting the cell expressing the first Cas9 domain with a first guide RNA and a fusion protein comprising: 1) a second Cas9 domain, wherein the second Cas9 domain when associated with a second guide RNA (gRNA) specifically binds to a target nucleic acid sequence and 2) a cytidine deaminase domain capable of deaminating a cytosine base in a single-stranded portion of the target nucleic acid sequence, or a nucleic acid sequence encoding the fusion protein. The first guide RNA when associated with the first Cas9 domain binds to a non-target nucleic acid sequence (e.g., a nucleic acid sequence different from the target nucleic acid sequence).

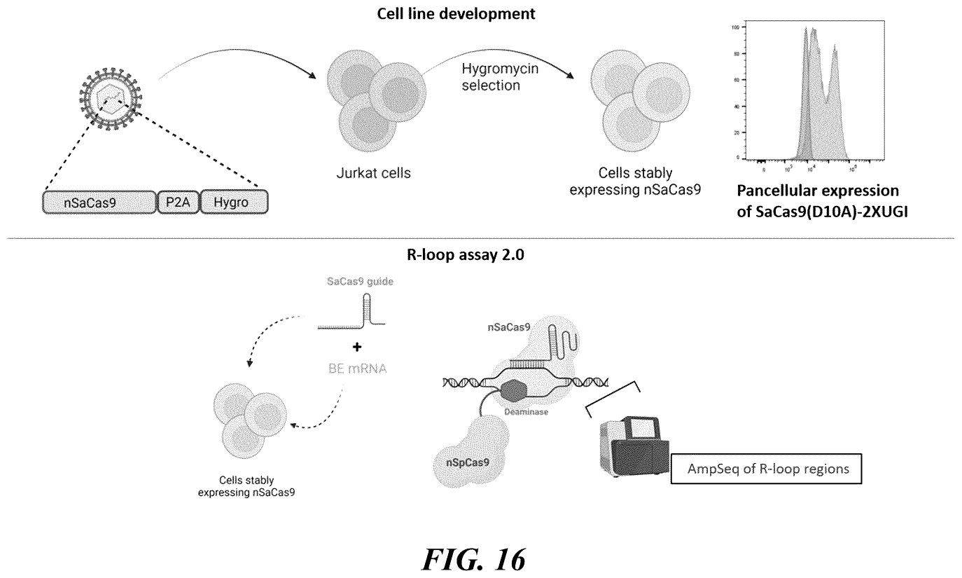

Upon binding to its target locus (e.g., the non-target nucleic acid sequence) in a DNA molecule, base pairing between the first gRNA and target DNA strand leads to displacement of a small segment of single-stranded DNA in an “R-loop”. DNA bases within the R-loop formed by the first Cas9 domain and the first gRNA may be modified by the deaminase domain of the fusion protein (see, for example, FIG. 16, the bottom panel).

The first Cas9 domain, the fusion protein and the guide RNAs can be introduced to the cell via any suitable transfection approach described herein and known in the art. In some embodiments, the first Cas9 domain, the first guide RNA, and the fusion protein can be co-transfected (e.g., transfected simultaneously) or transfected sequentially (e.g., one after another). In some embodiments, the method comprises contacting the cell with a plasmid or viral vector encoding a first Cas9 domain to produce a cell expressing the first Cas9 domain. In some embodiments, the cell is in vitro. The cell expressing the first Cas9 domain can be further transfected (e.g., via nucleofection) with a nucleic acid encoding a fusion protein disclosed herein and a first guide RNA capable of binding to the first Cas9.

In some embodiments, the first Cas9 domain can comprise a catalytically impaired Cas9. For example, the first Cas9 domain can comprise a truncated version of a nuclease domain or no nuclease domain at all. In some embodiments, the first Cas9 domain can comprise a Cas9 nickase, such as a SaCas9 nickase. In some embodiments, the first Cas9 domain can further comprise at least one UGI domain. For example, the first Cas9 domain can be fused to two UGI domains. The second Cas9 domain of the fusion protein can be any Cas9 domain described herein in the context of a base editing fusion protein. In some embodiments, the first Cas9 domain and the second Cas9 domain are different. For example, the first Cas9 domain and the second Cas9 domain can be derived from different organisms. The first Cas9 domain can comprise a SaCas9 domain while the second Cas9 domain can comprise a SpCas9 domain. In some embodiments, the first Cas9 domain and the second Cas9 can have different PAM specificities.

Following the transfection, genomic DNA of the cell can be extracted and analyzed using any suitable sequencing analysis tool known in the art. For example, amplicon sequencing of the R-loop regions induced by the first Cas9 domain can be performed to analyze the spurious off-target deamination activity.

In some embodiments, the base editing fusion proteins described herein have a spurious off-target deamination rate less than 5%, 4%, 3%, 2%, 1%, 0.5%, 0.4%, 0.3%, 0.2%, 0.1% or lower.

### Engineered Cells

Provided herein includes a method for producing an engineered cells and a population of genetically engineered cells prepared by the method.

The method can, for example, comprise providing a plurality of cells, delivering to the plurality of cells (a) a fusion protein described herein or a nucleic acid encoding the fusion protein and (b) at least one guide RNA targeting at least one target gene, genetically editing the at least one target gene, and producing one or more genetically engineered cells having at least one gene edit in the at least one target gene.

In some embodiments, the plurality of cells are immune cells. In some embodiments, the plurality of cells are T cells. The plurality of cells (e.g., T cells) can be derived from parent T cells (e.g., non-edited wild-type T cells) obtained from a suitable source, for example, one or more mammal donors. In some examples, the parent T cells are primary T cells (e.g., non-transformed and terminally differentiated T cells) obtained from one or more human donors. Alternatively, the parent T cells can be differentiated from precursor T cells obtained from one or more suitable donor or stem cells such as hematopoietic stem cells or inducible pluripotent stem cells (iPSC), which may be cultured in vitro.

T cells can be obtained from a number of sources, including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells are obtained from a unit of blood collected from a subject using any number of techniques known to the skilled person, such as sedimentation, e.g., FICOLL™ separation.

In some embodiments, T cells can be isolated from a mixture of immune cells (e.g., those described herein) to produce an isolated T cell population. For example, after isolation of peripheral blood mononuclear cells (PBMC), both cytotoxic and helper T lymphocytes can be sorted into naive, memory, and effector T cell subpopulations either before or after activation, expansion, and/or genetic modification.

A specific subpopulation of T cells, expressing one or more of the following cell surface markers: TCRab, CD3, CD4, CD8, CD27 CD28, CD38 CD45RA, CD45RO, CD62L, CD127, CD122, CD95, CD197, CCR7, KLRG1, MCH-I proteins and/or MCH-II proteins, can be further isolated by positive or negative selection techniques. In some embodiments, a specific subpopulation of T cells, expressing one or more of the markers selected from the group consisting of TCRab, CD4 and/or CD8, is further isolated by positive or negative selection techniques. In some embodiments, the engineered T cell populations do not express or do not substantially express one or more of the following markers: CD70, CD57, CD244, CD160, PD-1, CTLA4, HM3, and LAG3. In some embodiments, subpopulations of T cells can be isolated by positive or negative selection prior to genetic engineering and/or post genetic engineering.

In some embodiments, an isolated population of T cells can express one or more of the T cell markers, including, but not limited to a CD3+, CD4+, CD8+, or a combination thereof. In some embodiments, the T cells are isolated from a donor, or subject, and first activated and stimulated to proliferate in vitro prior to undergoing gene editing.

In some embodiments, the T cell population comprises primary T cells isolated from one or more human donors. Such T cells are terminally differentiated, not transformed, depend on cytokines and/or growth factors for growth, and/or have stable genomes. Alternatively, the T cells can be derived from stem cells (e.g., HSCs or iPSCs) via in vitro differentiation.

T cells from a suitable source can be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells can be activated and expanded for about, at least, at least about, at most, or at most about 4 hours, 6 hours, 12 hours, 24 hours, 1 day to 4 days, 1 day to 3 days, 1 day to 2 days, 2 days to 3 days, 2 days to 4 days, 3 days to 4 days, or 2 days, 3 days, or 4 days prior to introduction of the genome editing compositions into the T cells. In some embodiments, T cells are activated and expanded for about 4 hours, about 6 hours, about 12 hours, about 18 hours, about 24 hours, about 36 hours, about 48 hours, about 60 hours, or about 72 hours prior to introduction of the gene editing compositions into the T cells. In some embodiments, T cells are activated at the same time that genome editing compositions are introduced into the T cells. In some embodiments, the T cell population can be expanded and/or activated after the genetic editing as disclosed herein. T cell populations or isolated T cells generated by any of the gene editing methods described herein are also within the scope of the present disclosure.

The method herein described can further comprise delivering to the plurality of cells (e.g., T cells or precursor cells thereof described above) a nucleic acid encoding a chimeric antigen receptor (CAR). For example, the CAR can comprise an extracellular antigen binding domain specific to a tumor antigen, a co-stimulatory signaling domain of 4-1BB or CD28, and a cytoplasmic signaling domain of CD35. The tumor antigen can be CD19, BCMA, CD70, CD33, or PTK7. For example, the CAR can binds CD19 (anti-CD19 CAR) and the extracellular antigen binding domain in the anti-CD19 CAR is a single chain variable fragment (scFv) that binds CD19 (anti-CD19 scFv). In another example, the nucleic acid encoding a CAR can comprise an ectodomain that binds specifically to LIV1. In some embodiments, the ectodomain that binds specifically to LIV1 comprises an anti-LIV1 antigen-binding fragment, and optionally the anti-LIV1 antigen-binding fragment comprises an anti-LIV1 antibody.

The nucleic acid encoding a CAR can be delivered to the cells via conventional viral and non-viral based gene transfer methods known to a skilled person. In some embodiments, a nucleic acid encoding a CAR construct can be delivered to a cell using an adeno-associated virus (AAV) such as AAV6. In some embodiments, a nucleic acid encoding a CAR can be designed to insert into a genomic site of interest in the host T cells via a donor template. In some embodiments, a nucleic acid encoding a CAR (e.g., via a donor template, which can be carried by a viral vector such as an AAV vector) can be designed such that it can insert into a location within a TRAC gene to disrupt the TRAC gene in the engineered T cells and express the CAR polypeptide.

The method can comprise genetically editing one or more target genes, including the target genes described herein and known in the art, using the nucleic acid editing methods described. In some embodiments, the gRNA comprises two or more guide RNAs targeting two or more target genes and wherein the produced one or more genetically engineered cells have two or more gene edits in the two or more target genes.

The engineered cells can be produced by sequential targeting of the genes of interest. For example, in some embodiments, a first target gene can be edited first, followed by editing of a second, third, fourth and more target genes. In other embodiments, two or more target genes can be edited simultaneously. Accordingly, in some embodiments, the genetically engineered cells disclosed herein can be produced by multiple, sequential electroporation events with guide RNAs and the base editing fusion protein or complexes thereof targeting the genes of interest. In other embodiments, the engineered CAR cells disclosed herein can be produced by a single electroporation event with a complex comprising a base editing fusion protein and multiple gRNAs targeting the genes of interest.

In some embodiments, the at least one target gene is selected from the group consisting of the Regnase-1 (Reg1) gene, the Transforming Growth Factor Beta Receptor II (TGFBRII) gene, the TRAC gene, the beta-2-microglobulin (62M) gene, the CD70 gene, T cell receptor alpha chain constant region (TRAC) gene, or a combination thereof.

Provided herein also includes a population of engineered cells (e.g., immune cells such as T cells) prepared by the method described above. The population of engineered cells comprise one or more gene edits. In some embodiments, the engineered cells comprise two or more gene edits.

In some embodiments, at least 50%, 55%, 60%, 65%, 70%, 75%, 80%, 85%, 90%, 95%, 96%, 97%, 98%, 99%, 99.5% or more of the cells in the population of genetically engineered cells comprise at least two genome edits (e.g., two, three, four, five, six or more genome edits).

In some embodiments, at least 50% of a population of engineered cells may not express a detectable level of a target protein. For example, at least 55%, at least 60%, at least 70%, at least 75%, at least 80%, at least 85%, at least 90%, at least 95%, at least 98%, at least 99%, or at least 99.5% or more of the engineered cells may not express a detectable level of a target protein (e.g., β2M, TRAC, CD70, Reg1, and/or TGFBRII protein). In some embodiments, at least 55%, 60%, 65%, 70%, 75%, 80%, 85%, 90%, 95%, 96%, 97%, 98%, 99%, 99.5% or more of the cells in the population of genetically engineered cells may not express a detectable level of two or more target proteins (e.g., two, three, four, five, six or more target proteins).

In some embodiments, engineered cells (e.g., T cells) of the present disclosure exhibit at least 20% greater cellular proliferative capacity, relative to control cells. For example, engineered cells can exhibit about, at least, or at least about 25%, 30%, 35%, 40%, 45%, 50%, 55%, 60%, 65%, 70%, 75%, 80%, or 90% greater cellular proliferative capacity, relative to control cells. In some embodiments, engineered cells of the present disclosure exhibit 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% greater cellular proliferative capacity, relative to control cells.

In some embodiments, engineered cells of the present disclosure exhibit an at least 20% increase in cellular viability, relative to control cells. For example, engineered cells of the present disclosure can exhibit about, at least, or at least about 25%, 30%, 35%, 40%, 45%, 50%, 55%, 60%, 65%, 70%, 75%, 80%, 90% or a number between any two of the values, increase in cellular viability, relative to control cells. In some embodiments, engineered cells of the present disclosure exhibit a 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% increase in cellular viability, relative to control cells. The control cells can be engineered cells or unedited cells. In some embodiments, the control cells are cells edited using a gene editing strategy different from the ones described herein. The control cells can comprise one or more gene edits. In some embodiments, the control cells comprise one gene edit.

In some embodiments, fewer than 10%, 9%, 8%, 7%, 6%, 5%, 4%, 3%, 2%, 1%, 0.5%, 0.2% or 0.1% of the cells in the population of genetically engineered cells have an insertion, deletion, translocation or other DNA rearrangement.

### Pharmaceutical Compositions and Therapeutic Applications

Provided herein also include a pharmaceutical composition for carrying out the methods disclosed herein and related methods of using the base editing fusion protein, pharmaceutical compositions and cells described herein to prevent or treat a disease or disorder.

The composition can, for example, comprise (a) a fusion protein described herein or a nucleic acid sequence encoding the fusion protein and (b) one or more guide RNAs targeting one or more target genes. In some embodiments, a pharmaceutical composition is provided, the pharmaceutical composition comprising the composition and a pharmaceutical acceptable carrier or excipient.

In some embodiments, a composition described above can further have one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. In some embodiments, a composition can also include one or more components that can be used to facilitate or enhance the on-target binding or the cleavage of DNA by the endonuclease, or improve the specificity of targeting.

In some embodiments, any components of a composition are formulated with pharmaceutically acceptable excipients such as carriers, solvents, stabilizers, adjuvants, diluents, etc., depending upon the particular mode of administration and dosage form. In embodiments, guide RNA compositions are generally formulated to achieve a physiologically compatible pH, and range from a pH of about 3 to a pH of about 11, about pH 3 to about pH 7, depending on the formulation and route of administration. In some embodiments, the pH is adjusted to a range from about pH 5.0 to about pH 8.

Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

Physiologically tolerable carriers are well known in the art. Exemplary liquid carriers are sterile aqueous solutions that contain no materials in addition to the active ingredients and water or contain a buffer such as sodium phosphate at physiological pH value, physiological saline or both, such as phosphate-buffered saline. Aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition will depend on the nature of the disorder or condition and can be determined by standard clinical techniques.

In some embodiments, the compounds herein described (e.g., a base editing fusion protein or a nucleic acid encoding the base editing fusion protein and/or gRNAs) of a composition can be delivered via transfection such as calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof. In some embodiments, the composition is introduced to the cells via lipid-mediated transfection using a lipid nanoparticle.

In some embodiments, the compounds of the composition disclosed herein (e.g., the gRNA and the nucleic acid encoding the fusion protein) can be formulated in a liposome or lipid nanoparticle. In some embodiments, the compounds of the composition are formulated in a lipid nanoparticle (LNP). LNP is a non-viral delivery system that safely and effectively deliver nucleic acids to target organs (e.g., liver). The term “lipid nanoparticle” refers to a nanoscopic particle composed of lipids having a size measured in nanometers (e.g., 1-5,000 nm). In some embodiments, the lipids comprised in the lipid nanoparticles comprise cationic lipids and/or ionizable lipids. Any suitable cationic lipids and/or ionizable lipids known in the art can be used to formulate LNPs for delivery of gRNA and Cas endonuclease to the cells. Exemplary cationic lipids include one or more amine group(s) bearing positive charge. In some embodiments, the cationic lipids are ionizable such that they can exist in a positively charged or neutral from depending on pH. In some embodiments, the cationic lipid of the lipid nanoparticle comprises a protonatable tertiary amine head group that shows positive charge at low pH. The lipid nanoparticles can comprise further one or more neutral lipids (e.g., Distearoylphosphatidylcholine (DSPC), 1,2-Dioleoyl-sn-glycero-3-phosphocholine (DOPC), 1,2-Dimyristoyl-sn-glycero-3-phosphoethanolamine (DMPE), 1,2-Dipalmitoyl-sn-glycero-3-phosphorylethanolamine (DPPE) etc. as a helper lipid), charged lipids, steroids, and polymers conjugated lipids.

The lipid nanoparticles can have a mean diameter of about, at least, at least about, at most or at most about 30 nm, 40 nm, 50 nm, 60 nm, 70 nm, 80 nm, 90 nm, 100 nm, 110 nm, 120 nm, 130 nm, 140 nm, 150 nm, or a number or a range between any of these values. In some embodiments, the lipid nanoparticle particle size is about 50 to about 100 nm in diameter, or about 70 to about 90 nm in diameter, or about 55 to about 95 nm in diameter.

the compounds of the composition described herein are encapsulated in the lipid portion of the lipid nanoparticle or an aqueous space enveloped by some or all of the lipid portion of the lipid nanoparticle. The encapsulation can be full encapsulation, partial encapsulation, or both. In some embodiments, the nucleic acid and/or polypeptides are fully or substantially encapsulated (e.g., greater than 90% of the RNA) in the lipid nanoparticle.

In some embodiments, one or more compounds herein described are associated with a liposome or lipid nanoparticle via a covalent bond or non-covalent bond. In some embodiments, any of the compounds in the composition can be separately or together contained in a liposome or lipid nanoparticle.

The composition and/or pharmaceutical composition herein described can be administered to a subject in need thereof to prevent or treat a disease or disorder. Accordingly, the present disclosure also provides a method of preventing or treating a disease or disorder in a subject in need thereof. The method can comprise administering to the subject a therapeutically effective amount of the composition or pharmaceutical composition described herein, wherein the one or more target gene is associated with the disease or disorder.

A subject can be any subject for whom diagnosis, treatment, or therapy is desired. In some embodiments, the subject is a mammal. In some embodiments, the subject is a human. In some embodiments, the subject is suspected to have, having or diagnosed to have a disease or disorder.

Any suitable administration route capable of delivering the composition can be used herein. In some embodiments, the pharmaceutical composition thereof can be administered by aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intracardiac injection, intraperitoneal injection, intrathecal injection, intraventricular injection, intracerebroventricular injection, intradermal injection, or any combination thereof. The administration can be local or systemic. Systemic administration refers to the administration of a population of cells other than directly into a target site, tissue, or organ, such that it enters the subject's circulatory system and, thus, is subject to metabolism and other like processes. Systemic administration can include enteral and parenteral administration. In some embodiments, more than one administration can be employed to achieve the desired level of gene expression over a period of various intervals, e.g., daily, weekly, monthly, or yearly. In some embodiments, the route is intravenous.

The pharmaceutical composition thereof can be administered to a subject in need thereof at a pharmaceutically effective amount. The amount of the pharmaceutical composition can result in a desired reduction or loss of function in one or more gene products.

The disease or disorder can be, for example, a disease associated with a gene having a point mutation (e.g., a T to C point mutation) and the methods and compositions described herein can correct the point mutation therefore preventing or treating the disease or disorder. Examples of genes comprising a pathogenic T to C point mutation associated with a disease or disorder are disclosed herein. Additional suitable gene sequences that can be corrected with the methods and compositions herein described will be apparent to those of skill in the art based on this disclosure.

In some embodiments, the disease or disorder is cancer. Non-limiting examples of cancers that can be treated as provided herein include: breast cancer, e.g., estrogen receptor-positive breast cancer, prostate cancer, squamous tumors, e.g., of the skin, bladder, lung, cervix, endometrium, head neck, and biliary tract, and neuronal tumors. In some embodiments, the methods comprise delivering the CAR T cells of the present disclosure to a subject having cancer, including, breast cancer, e.g., estrogen receptor-positive breast cancer, prostate cancer, squamous tumors, e.g., of the skin, bladder, lung, cervix, endometrium, head neck, and biliary tract, and/or neuronal tumors.

The base editing fusion proteins, compositions, engineered T cells, methods and kits disclosed herein can be used to treat various types of cancer, including but are not limited to, melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer (NSCLC) and small-cell lung cancer (SCLC)), esophageal cancer, squamous cell carcinoma of the head and neck, liver cancer, ovarian cancer, cervical cancer, thyroid cancer, glioblastoma, glioma, leukemia, lymphoma, and other neoplastic malignancies. Additionally, the disease or condition provided herein includes refractory or recurrent malignancies whose growth may be inhibited using the methods and compositions disclosed herein. In some embodiments, the cancer is carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, or a combination thereof. In some embodiments, the cancer is carcinoma, squamous carcinoma (e.g., cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, the cancer is sarcomata (e.g., myogenic sarcoma), leukosis, neuroma, melanoma, and lymphoma.

The cancer can include pancreatic cancer, gastric cancer, ovarian cancer, uterine cancer, breast cancer, prostate cancer, testicular cancer, thyroid cancer, nasopharyngeal cancer, non-small cell lung (NSCLC), glioblastoma, neuronal, soft tissue sarcomas, leukemia, lymphoma, melanoma, colon cancer, colon adenocarcinoma, brain glioblastoma, hepatocellular carcinoma, liver hepatocholangiocarcinoma, osteosarcoma, gastric cancer, esophagus squamous cell carcinoma, advanced stage pancreas cancer, lung adenocarcinoma, lung squamous cell carcinoma, lung small cell cancer, renal carcinoma, intrahepatic biliary cancer, and a combination thereof. In some embodiments, the cancer is breast cancer, prostate cancer, squamous tumor cancer, neuronal tumor cancer, or a combination thereof. In some embodiments, the cancer comprises cancer cells expressing LIV1.

The cancer can be a solid tumor, a liquid tumor, or a combination thereof. In some embodiments, the cancer is a solid tumor, including but are not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, Merkel cell carcinoma, brain and central nervous system cancers, and any combination thereof. In some embodiments, the cancer is a liquid tumor. In some embodiments, the cancer is a hematological cancer. Non-limiting examples of hematological cancer include diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), and multiple myeloma (“MM”).

In some embodiments, the base editing fusion proteins, compositions, engineered T cells, methods and kits disclosed herein can be used to treat arteriosclerosis, atherosclerosis, cardiovascular diseases, coronary heart disease, diabetes, diabetes mellitus, non-insulin-dependent diabetes mellitus, fatty liver, hyperinsulinism, hyperlipidemia, hypertriglyceridemia, hypobetalipoproteinemias, inflammation, insulin resistance, metabolic diseases, obesity, malignant neoplasm of mouth, lipid metabolism disorders, lip and oral cavity carcinoma, dyslipidemias, metabolic syndrome x, hypotriglyceridemia, opitz trigonocephaly syndrome, ischemic stroke, hypertriglyceridemia result, hypobetalipoproteinemia familial 2, familial hypobetalipoproteinemia, and ischemic cerebrovascular accident.

In some embodiments, the base editing fusion proteins, compositions, engineered T cells, methods and kits disclosed herein can be used to treat a metabolic disease, a lipid metabolism disease, obesity, atherosclerosis, hyperfattyacidemia, metabolic syndrome, dyslipidemia, hypobetalipoproteinemia, familial hypercholesterolemia (including homozygous familial hypercholesterolemia (HoFH) and heterozygous familial hypercholesterolemia (HeFH)), hypertriglyceridemia, familial combined hyperlipidemia, familial chylomicronemia syndrome, multifactorial chylomicronemia syndrome, familial combined hyperlipidemia (FCHL), metabolic syndrome (MetS), nonalcoholic fatty liver disease (NAFLD), elevated lipoprotein (a), elevated lipids such as total cholesterol, triglycerides, LDLs, HDLs, and/or other non-HDLs in the blood, or a combination thereof. NAFLD can be hepatic steatosis or steatohepatitis. The diabetes can be type 2 diabetes or type 2 diabetes with dyslipidemia. Dyslipidemia can be hyperlipidemia, for example hypercholesterolemia, hypertriglyceridemia, or both.

In some embodiments, the methods and compositions provided herein can comprise a guide RNA targeting a LPA gene (including, e.g., LPA gene variants associated with increased cardiovascular disease risk and/or increased Lp(a) expression). The LPA gene encodes the apolipoprotein (a) protein of lipoprotein (a) (Lp(a)) in a cell genome to modulate (e.g., decrease) the expression, function, or activity of the Lp(a) in the cell. The term “LPA gene” as used herein includes the genomic region encompassing the LPA regulatory promoters and enhancer sequences as well as the coding sequence.

Accordingly, in some embodiments, the base editing fusion proteins, compositions, engineered T cells, methods and kits disclosed herein can be used to treat a cardiovascular disease or disorder. As used herein, the term “cardiovascular disease” refers to a disorder of the heart and blood vessels, and includes disorders of the arteries, veins, arterioles, venules, and capillaries. In some embodiments, the cardiovascular disease is stroke, myocardial infarction, atherosclerosis, familial hypercholesterolemia, atherosclerosis, thrombosis, calcific aortic valve disease, coronary artery disease, peripheral arterial disease, cerebrovascular disease, renal artery stenosis, aortic aneurysm, cardiomyopathy, hypertensive heart disease, heart failure, pulmonary heart disease, congenital heart disease, or rheumatic heart disease. In some embodiments, the base editing fusion proteins, compositions, engineered T cells, methods and kits disclosed herein can be used to treat calcific aortic valve disease, myocardial infarctions, coronary heart disease, atherosclerosis, thrombosis, stroke or a combination thereof.

The methods and compositions herein described can reduce the plasma low-density lipoprotein (LDL) levels such as the plasms Lp(a) levels, therefore reducing the risk of cardiovascular disease, such as the risk of heart attack, stroke, blood clots, fatty build-up in veins and other coronary artery disease, the likelihood of mortality related to cardiovascular events, or a combination thereof. In some embodiments, the methods and compositions herein described can reduce or relieve one or more symptoms of the cardiovascular disease. In some embodiments, the plasma Lp(a) level in the subject following carrying out the method can be reduced by about, at least, or at least about 50%, 60%, 70%, 80%, 90%, 95%, 98%, 99%, or a number or a range between any of these values.

In some embodiments, the method can comprise administering to a subject an engineered cell herein described or a population of the engineered cells.

The step of administering can include introducing (e.g., transplantation) the cells, e.g., an engineered T cell or a population thereof described herein, into a subject, by a method or route that results in at least partial localization of the introduced cells at a desired site, such as tumor, such that a desired effect(s) is produced. Engineered T cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. For example, in some aspects described herein, an effective amount of engineered T cells is administered via a systemic route of administration, such as an intraperitoneal or intravenous route.

In some embodiments, an engineered T cell population being administered according to the methods described herein comprises allogeneic T cells obtained from one or more donors. Allogeneic refers to a cell, cell population, or biological samples comprising cells, obtained from one or more different donors of the same species, where the genes at one or more loci are not identical to the recipient. For example, an engineered T cell population, being administered to a subject can be derived from one or more unrelated donors, or from one or more non-identical siblings. In some embodiments, syngeneic cell populations can used, such as those obtained from genetically identical donors, (e.g., identical twins). In some embodiments, the cells are autologous cells; that is, the engineered T cells are obtained or isolated from a subject and administered to the same subject, i.e., the donor and recipient are the same. A donor as used herein is an individual who is not the subject being treated. A donor is an individual who is not the patient. In some embodiments, a donor is an individual who does not have or is not suspected of having the cancer being treated. In some embodiments, multiple donors, e.g., two or more donors, are used.

In some embodiments, an engineered T cell population being administered according to the methods described herein does not induce toxicity in the subject, e.g., the engineered T cells do not induce toxicity in non-cancer cells. In some embodiments, an engineered T cell population being administered does not trigger complement mediated lysis, or does not stimulate antibody-dependent cell mediated cytotoxicity (ADCC).

An effective amount refers to the amount of a population of engineered T cells needed to prevent or alleviate at least one or more signs or symptoms of a medical condition, and relates to a sufficient amount of a composition to provide the desired effect, e.g., to treat a subject having a medical condition.

In some embodiments, an effective amount of cells (e.g., engineered T cells) comprises about, at least or at least about 10² cells, 5×10² cells, 10³ cells, 5×10³ cells, 10⁴ cells, 5×10⁴ cells, 10⁵ cells, 2×10⁵ cells, 3×10⁵ cells, 4×10⁵ cells, 5×10⁵ cells, 6×10⁵ cells, 7×10⁵ cells, 8×10⁵ cells, 9×10⁵ cells, 1×10⁶ cells, 2×10⁶ cells, 3×10⁶ cells, 4×10⁶ cells, 5×10⁶ cells, 6×10⁶ cells, 7×10⁶ cells, 8×10⁶ cells, 9×10⁶ cells, 10×10⁶ cells, 12×10⁶ cells, 14×10⁶ cells, 16×10⁶ cells, 18×10⁶ cells, 20×10⁶ cells, 25×10⁶ cells, 30×10⁶ cells, or a number between any two of the values. The cells are derived from one or more donors, or are obtained from an autologous source. In some embodiments described herein, the cells are expanded in culture prior to administration to a subject in need thereof.

### Combinational Cancer Therapy

The fusion proteins, complexes, compositions, engineered cells, methods, and kits disclosed herein can be used with additional cancer therapeutics or therapy to treat cancer. In some embodiments, the treatment can comprise administration of at least one additional cancer therapeutics or cancer therapy. The treatment can comprise administration a therapeutically effective amount of at least one additional cancer therapeutics or cancer therapy. The compositions, complexes, and engineered cells herein described and the cancer therapeutics or cancer therapy can, for example, co-administered simultaneously or sequentially. Examples of the cancer therapies include, but are not limited to, surgery, chemotherapy, radiation therapy, hormonal therapy, immunotherapy, complementary or alternative therapy, and any combination thereof.

The additional therapeutic agents can comprises one or more chemotherapeutics, including but are not limited to, mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples of the additional therapeutic agents include chemotherapeutic agents, cytotoxic agents, and non-peptide small molecules such as Gleevec® (Imatinib Mesylate), Kyprolis® (carfilzomib), Velcade® (bortezomib), Casodex (bicalutamide), Iressa® (gefitinib), venetoclax, and Adriamycin. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; retinoic acid; esperamicins; capecitabine; and pharmaceutically acceptable salts, acids or derivatives of any of the above.

In some embodiments, the one or more additional agents comprise anti-hormonal agents capable of regulating or inhibiting hormone action on tumors such as anti-estrogens including for example tamoxifen, (Nolvadex™), raloxifene, aromatase inhibiting 4 (5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO).

In some embodiment, the one or more additional therapeutic agents that can be administered to the subject receiving, has received, or will receive, the administration of the engineered T cells disclosed herein comprise currently prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126, Zosuquidar or a combination thereof.

The methods, compositions and kits disclosed herein can be, in some embodiments, used in combination with radiation therapy for inhibiting abnormal cell growth or treating a cancer. Non-limiting examples of radiation therapy include, but are not limited to, external-beam therapy, internal radiation therapy, implant radiation, stereotactic radiosurgery, systemic radiation therapy, radiotherapy and permanent or temporary interstitial brachytherapy.

In some embodiments, the base editing protein complexes, compositions, engineered cells, methods or kits disclosed herein is used in combination with one or more of anti-angiogenesis agents, chemotherapeutic agents, anti-neoplastic agents, steroids, signal transduction inhibitors, antiproliferative agents, glycolysis inhibitors, and autophagy inhibitors. The anti-angiogenesis agents can be MMP-2 (matrix-metalloproteinase 2) inhibitors, MMP-9 (matrix-metalloprotienase 9) inhibitors, and COX-11 (cyclooxygenase 11) inhibitors. Anti-angiogenesis agents include, for example, rapamycin, temsirolimus (CCI-779), everolimus (RAD001), sorafenib, sunitinib, and bevacizumab. Non-limiting examples of COX-II inhibitors include alecoxib, valdecoxib, and rofecoxib. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tegafur, tazarotene, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), or valspodar.

Examples of anti-angiogenic agent include, but are not limited to, ERBITUX™ (IMC-C225), KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as AVASTIN™ or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix (panitumumab), IRESSA™ (gefitinib), TARCEVA™ (erlotinib), anti-Ang1 and anti-Ang2 agents (e.g., antibodies or antigen binding regions specifically binding thereto or to their receptors, e.g., Tie2/Tek), anti-Tie2 kinase inhibitory agents (e.g., antibodies or antigen binding regions that specifically bind thereto), Campath, IL-8, B-FGF, Tek antagonists, anti-TWEAK agents (e.g., specifically binding antibodies or antigen binding regions, or soluble TWEAK receptor antagonists), ADAM distintegrin domain to antagonize the binding of integrin to its ligands, specifically binding anti-eph receptor and/or anti-ephrin antibodies or antigen binding regions, anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Autophagy inhibitors include, but are not limited to, chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine.

Non-limiting chemotherapeutic agents include, natural products such as vinca alkaloids (e.g., vinblastine, vincristine, and vinorelbine), paclitaxel, epidipodophyllotoxins (e.g., etoposide and teniposide), antibiotics (e.g., dactinomycin (actinomycin D), daunorubicin, doxorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTor inhibitors (e.g., temsirolimus, everolimus, ridaforolimus, and sirolimus), KSP (Eg5) inhibitors (e.g., Array 520), DNA binding agents (e.g., Zalypsis), PI3K delta inhibitor (e.g., GS-1101 and TGR-1202), PI3K delta and gamma inhibitor (e.g., CAL-130), multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNTO328), telomerase inhibitors (e.g., GRN 163L), aurora kinase inhibitors (e.g., MLN8237), cell surface monoclonal antibodies (e.g., anti-CD38 (HUMAX-CD38), anti-CS1 (e.g., elotuzumab), HSP90 inhibitors (e.g., 17 AAG and KOS 953), PI3K/Akt inhibitors (e.g., perifosine), Akt inhibitor (e.g., GSK-2141795), PKC inhibitors (e.g., enzastaurin), FTIs (e.g., Zarnestra™), anti-CD138 (e.g., BT062), Torc1/2 specific kinase inhibitor (e.g., INK128), kinase inhibitor (e.g., GS-1101), ER/UPR targeting agent (e.g., MKC-3946), cFMS inhibitor (e.g., ARRY-382), JAK1/2 inhibitor (e.g., CYT387), PARP inhibitor (e.g., olaparib and veliparib (ABT-888)), BCL-2 antagonist. Other chemotherapeutic agents may include mechlorethamine, camptothecin, ifosfamide, tamoxifen, raloxifene, gemcitabine, navelbine, sorafenib, or any analog or derivative variant of the foregoing.

Non-limiting examples of steroids include 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts and/or derivatives thereof. In a particular embodiment, the compounds of the present invention can also be used in combination with additional pharmaceutically active agents that treat nausea. Examples of agents that can be used to treat nausea include: dronabinol; granisetron; metoclopramide; ondansetron; and prochlorperazine; or a pharmaceutically acceptable salt thereof.

In some embodiments, the one or more additional therapeutic agents that are administered to the subject comprises one or more PD-1 antagonists, PD-L1 antagonists, EGFR inhibitors, MEK inhibitors, PI3K inhibitors, AKT inhibitors, TOR inhibitors, Mcl-1 inhibitors, BCL-2 inhibitors, SHP2 inhibitors, proteasome inhibitors, and immune therapies, including monoclonal antibodies, immunomodulatory imides (IMiDs), anti-PD-1, anti-PDL-1, anti-CTLA4, anti-LAG1, and anti-OX40 agents, GITR agonists, CAR-T cells, and BiTEs. Proteasome inhibitors include, but are not limited to, Kyprolis® (carfilzomib), Velcade® (bortezomib), and oprozomib. Monoclonal antibodies include, but are not limited to, Darzalex® (daratumumab), Herceptin® (trastuzumab), Avastin® (bevacizumab), Rituxan® (rituximab), Lucentis® (ranibizumab), and Eylea® (aflibercept).

In some embodiments, the cancer is breast cancer and the additional cancer therapies include surgery (e.g., breast conserving surgery or mastectomy), chemotherapy, radiation, hormonal therapy, HER2 targeted therapies and other target therapies (e.g., monoclonal antibodies, TKIs, cyclin-dependent kinase inhibitors, mTOR inhibitors, PARP inhibitors, PD-L1 inhibitor). In some embodiments, the additional therapeutic agents can include Trastuzumab, Pembrolizumab, Capecitabine, Atezolizumab, Ipatasertib, Bevacizumab, Cobimetinib, Gemcitabine, Carboplatin, Eribulin, or a combination thereof.

### Kits

Provided herein also includes kits for use in producing the base editing fusion proteins alone or in complex with a gRNA, the compositions, and the engineered cells and carrying out the methods described herein for therapeutic uses.

In some embodiments, a kit provide herein comprises components for performing base editing of one or more target sequences. A kit can comprise a base editing fusion protein herein described or a nucleic acid sequence encoding the base editing fusion protein, and one or more guide RNAs targeting the one or more target sequences. A kit can also comprises a nucleic acid construct comprising (a) a nucleotide sequence encoding a base editing fusion protein as provided herein; and (b) a heterologous promoter that drives expression of the sequence of (a). A kit can comprise a complex of the base editing fusion protein bound with a gRNA. A kit can also comprise cells comprising a deaminase protein, a fusion protein, a nucleic acid molecule encoding the fusion protein, a complex comprising the fusion protein bound with a gRNA, and/or a vector as provided herein. Components of a kit can be in separate containers, or combined in a single container.

Any kit described above can further comprise one or more additional reagents selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. A kit can also comprise one or more components that can be used to facilitate or enhance the on-target binding or the cleavage of DNA by the endonuclease, or improve the specificity of targeting.

In some embodiments, a kit can further include instructions for using the components of the kit to practice the methods described herein. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g., CD-ROM, diskette, flash drive, etc. In some instances, the actual instructions are not present in the kit, but means for obtaining the instructions from a remote source (e.g., via the Internet), can be provided. An example of this embodiment is a kit that includes a web address where the instructions can be viewed and/or from which the instructions can be downloaded. As with the instructions, this means for obtaining the instructions can be recorded on a suitable substrate.

### EXAMPLES

Some aspects of the embodiments discussed above are disclosed in further detail in the following examples, which are not in any way intended to limit the scope of the present disclosure.

### Example 1

### Analyses of Cytosine Base Editors Comprising Deaminases from Different Species

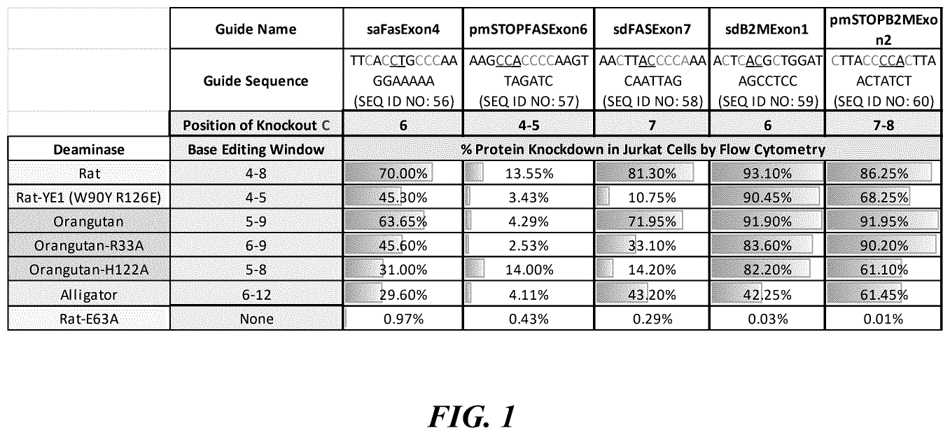

Five additional previously described APOBEC1 genes were chosen to evaluate in a base editor model (Table 1). Seven cytosine base editor mRNAs in total (including Rat APOBEC1 (protein sequence: SEQ ID NO: 87; nucleic acid sequence: SEQ ID NO: 88) and Rat-E63A APOBEC1) were produced by in vitro transcription (Table 1). These base editor mRNAs were introduced into Jurkat cells in conjunction with sgRNAs designed to create functional knockouts at the FAS and β2M loci, and the degree of β2M and FAS knockdown was determined by flow cytometry (FIG. 1). The CBE harboring the wild type Rat APOBEC1 gene showed the highest degree of protein knockdown for each locus. The protein knockdown capability of the CBE with wild type Orangutan APOBEC1 gene, though slightly lower, was comparable to what is observed in the wild type Rat APOBEC1 CBE. APOBEC1s harboring point mutations intended to enhance CBE fidelity by reducing spurious deamination, whether in the Rat (YE1) or the Orangutan APOBEC1 gene (R33A, H122A), demonstrated a reduced proficiency for on-target editing. The wild type APOBEC1 gene from the American Alligator also demonstrated lower knockdown capability compared to Orangutan and Rat, with the catalytically inactive Rat APOBEC1-E63A deaminase demonstrating no protein knockdown. C→T conversion rates were analyzed by Amplicon-Seq to show that C→T conversion rates were in concordance with the degree of protein knockdown for each respective CBE (Table 2).

To evaluate spurious off-target deamination (in trans activity), the R-loop assay that is described in Doman et al. 2020; Yu et al. 2020 was modified (see, for example, FIG. 16). Briefly, a catalytically impaired SaCas9 bound to an SaCas9 guide creates an R-loop at targeted loci, exposing ssDNA for the potential of guide-independent CBE deamination which is then quantitated by amplicon-seq. While previously described R-loop assays involved transfecting plasmids encoding catalytically impaired SaCas9, the assay used herein includes transfecting mRNAs encoding SaCas9 nickase fused to two UGI domains (e.g., SaCas9(D10A)-2XUGI). In some instances, SaCas9(D10A)-2XUGI construct was subcloned into an IVT vector and SaCas9(D10A)-2XUGI mRNA was produced. SpCas9 CBE mRNA was co-nucleofected with SaCas9(D10A)-2XUGI mRNA and SaCas9 sgRNAs into Jurkat cells. After >48 hours, cells were harvested, genomic DNA was isolated, and levels of C→T conversion were examined by amplicon seq. It was observed that wild type Rat APOBEC1 demonstrates the highest degree of spurious off-target deamination, as previously reported (Table 3), while wild type Orangutan APOBEC1 displays a significantly lower level by comparison. CBEs with APOBEC1 genes from wild type American Alligator or engineered variants of Rat and Orangutan displayed the lowest degree of spurious deamination amongst catalytically active constructs. The results indicated that the CBE comprised of the wild-type Orangutan APOBEC1 is better suited than the rat APOBEC1 for efficient on-target cytosine base editing while mitigating the degree of spurious deamination events and is used herein as a benchmark.

TABLE 1
Select APOBEC1 deaminases for analyses

| Common Name | Species | Mutant | Reference |
|---|---|---|---|
| Rat | Rattus norvegicus | Wild Type | (Komor et al. 2016) |
| Rat | Rattus norvegicus | E63A (catalytically | (Chieca et al. 2018) |
| | | inactive) | |
| Rat | Rattus norvegicus | YE1 (W90Y/R126E) | (Kim et al. 2017) |
| Orangutan | Pongo pygmaeus | Wild Type | (Yu et al. 2020) |
| Orangutan | Pongo pygmaeus | R33A | (Grunewald et al. 2019) |
| Orangutan | Pongo pygmaeus | H122A | (Yu et al. 2020) |
| American Alligator | Alligator mississippiens | Wild Type | (Yu et al. 2020) |

TABLE 2
C to T conversion rates by Amplicon-Seq at on-target loci

| Guide | B2M SD Ex.1 | B2MEx2pmSTOP | pmSTOPFASExon6 | saFASExon4 | sdFASExon7 |
|---|---|---|---|---|---|
| Rat | 63.9% | 81.1% | 36.6% | 65.3% | 74.6% |
| Rat- | 76.9% | 80.5% | 18.6% | 51.6% | 20.8% |
| YE1(W90Y | | | | | |
| R126E) | | | | | |
| Orangutan | 71.1% | 82.4% | 29.1% | 59.1% | 66.4% |
| Orangutan- | 77.3% | 87.5% | 21.2% | 51.0% | 45.2% |
| R33A | | | | | |
| Orangutan- | 78.1% | 78.7% | 24.3% | 42.4% | 24.8% |
| H122A | | | | | |
| Alligator | 58.7% | 71.0% | 12.1% | 44.4% | 45.4% |
| Rat-E63A | 0.1% | 0.2% | 0.0% | 0.0% | 0.0% |

TABLE 3
Spurious off-target deamination by R-loop Assay for Constructs

| Guide | Doman 3 | Doman 5 | Doman 6 | |
|---|---|---|---|---|
| Guide | GTGTCAGGTAATGTG | TCTGCTTCTCCAGC | GATGTTCCAATCA | Total |
| Sequence | CTAAACA | CCTGGC | GTACGCA | C→T |
| | (SEQ ID NO: 84) | (SEQ ID NO: 85) | (SEQ ID NO: 86) | |
| Rat | 0.22% | 19.13% | 12.83% | 32.18% |
| Rat- | 0.08% | 0.36% | 0.86% | 1.30% |
| YE1(W90Y | | | | |
| R126E) | | | | |
| Orangutan | 0.20% | 1.67% | 3.28% | 5.15% |
| Orangutan- | 0.07% | 0.61% | 0.74% | 1.42% |
| R33A | | | | |
| Orangutan- | 1.43% | 0.64% | 0.31% | 2.38% |
| H122A | | | | |
| Alligator | 0.12% | 0.20% | 0.23% | 0.55% |
| Rat-E63A | 0.08% | 0.30% | 0.23% | 0.61% |

Evaluation of APOBEC1 Deaminases from Select Species

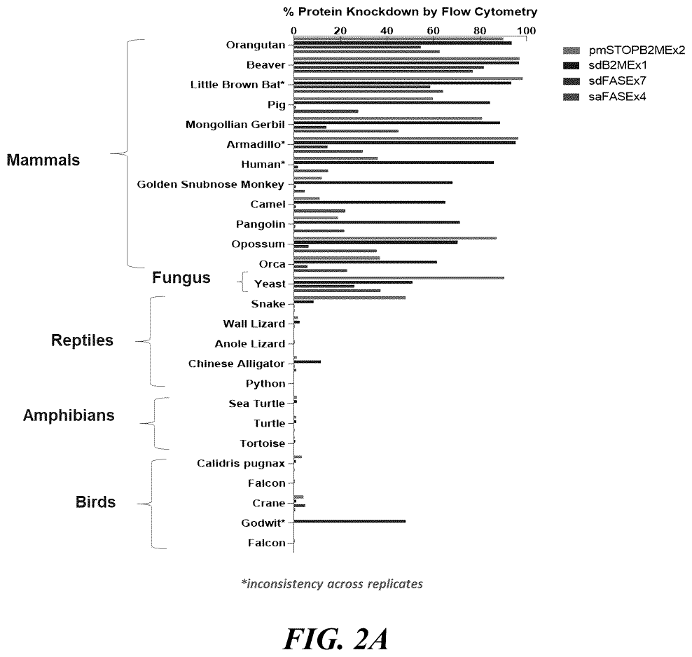

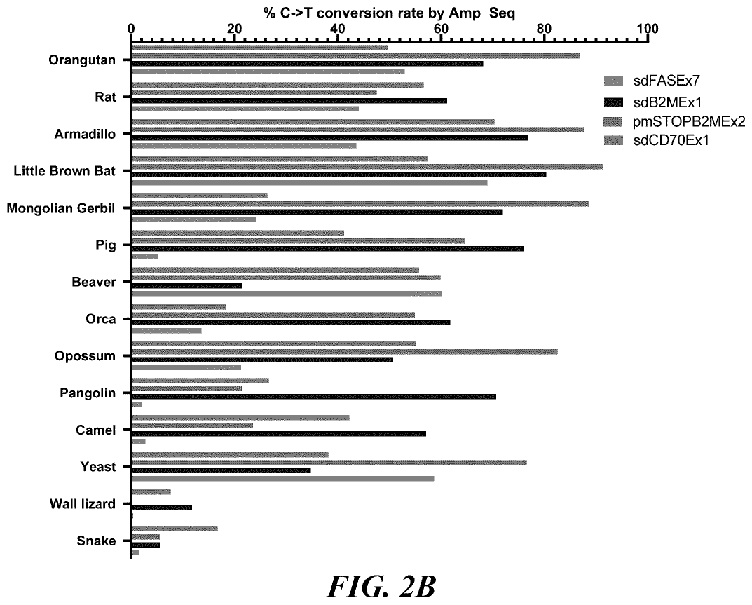

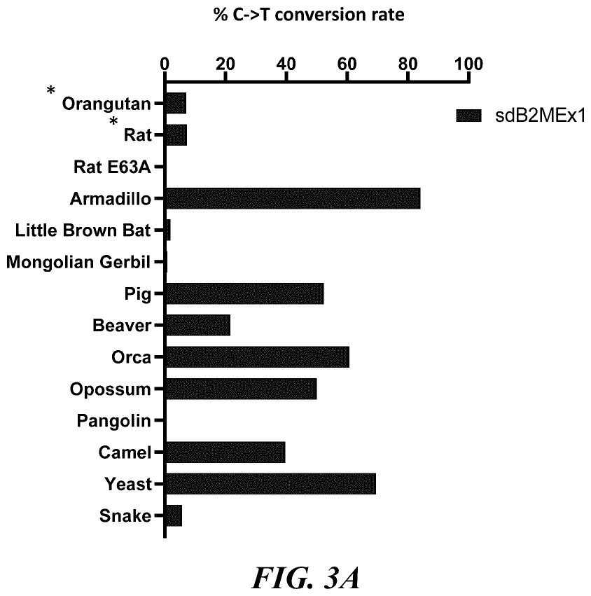

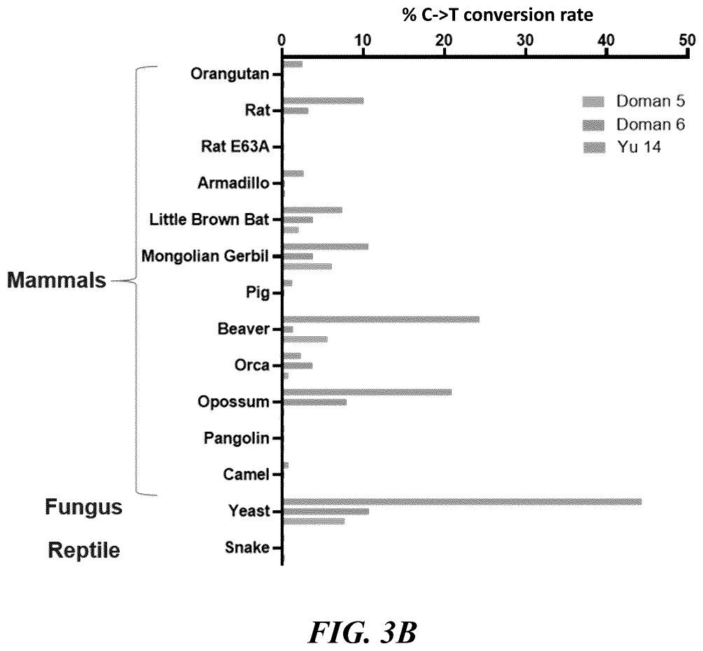

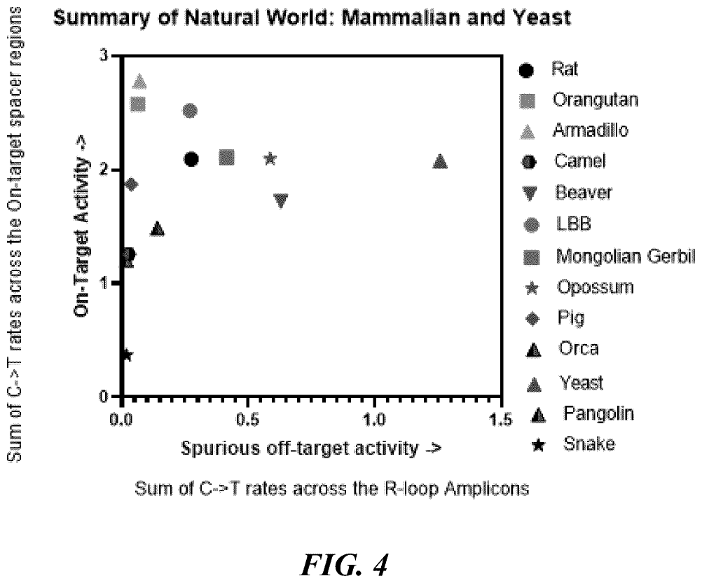

Several deaminases were evaluated to identify a deaminase with similar or better proficiency/fidelity to that observed in wild type Orangutan deaminase in the context of a CBE. A series of deaminase genes from numerous mammalian, reptilian, amphibian, avian, and fungal species (Table 4) were evaluated. Using the on-target and off-target (R-loop) evaluation methods described above, the editing proficiency of these CBEs was profiled. APOBEC1s from amphibians, reptiles, and birds displayed low efficiency of on-target editing by flow cytometry whereas mammalian APOBEC1 and the S. cerevisiae FCY1 genes were significantly higher to a level suitable for gene editing (FIG. 2A). The CBEs harboring mammalian APOBEC1 and yeast FCY1 deaminases for on-target activity were reevaluated by flow cytometry and amplicon-seq, and spurious off-target activity was examined via the R-loop assay (FIGS. 2A-B and 3A-B). As was observed in the previous Example, deaminases comprised of Rat APOBEC1 showed higher levels of spurious deamination compared to Orangutan (FIG. 3B). High levels of spurious deamination from base editors with deaminases from the Little Brown Bat, Beaver, Opossum, and the yeast were also observed (FIG. 4). The CBE harboring APOBEC1 from the Nine Banded Armadillo had relatively low off-target deamination with high on-target activity (FIGS. 3A-3B, 4). FIG. 4 summarizes the findings from this set of studies.

TABLE 4
Deaminases for examination

| | Deaminase | Species | Common Name |
|---|---|---|---|
| Mammalian | APOBEC1 | Castor canadiensis | Beaver |
| Mammalian | APOBEC1 | Monodelphis domestica | Gray short-tailed opossum |
| Mammalian | APOBEC1 | Myotis lucifugus | Little brown bat |
| Mammalian | APOBEC1 | Rhinopithecus roxellana | Golden snub-nosed monkey |
| Mammalian | APOBEC1 | Sus scrofa | Pig (e.g., APOBEC1 having |
| | | | an amino acid sequence of |
| | | | SEQ ID NO: 89 and a |
| | | | nucleic acid sequence of |
| | | | SEQ ID NO: 90) |
| Mammalian | APOBEC1 | Dasypus novemcinctus | Nine-banded armadillo |
| Mammalian | APOBEC1 | Orcinus orca | Orca |
| Mammalian | APOBEC1 | Manis pentadactyla | Chinese pangolin |
| Mammalian | APOBEC1 | Meriones unguiculatus | Mongolian gerbil |
| Mammalian | APOBEC1 | Camelus dromedarius | Arabian (one-humped) |
| | | | camel |
| Mammalian | APOBEC1 | Homo Sapiens | Human |
| Avian | APOBEC1 | Limosa lapponica baueri | Bar-tailed godwit |
| Avian | APOBEC1 | Calidris pugnax | Ruff (sandpiper) |
| Avian | APOBEC1 | Balearica regulorum | Grey crowned crane |
| | | gibbericeps | |
| Avian | APOBEC1 | Falco peregrinus | Peregrin falcon |
| Fungal | FCY1 | Saccharomyces cerevisiae | Yeast |
| Reptile | APOBEC1 | Crotalus tigris | Tiger rattlesnake |
| Reptile | APOBEC1 | Python bivittatus | Burmese python |
| Reptile | APOBEC1 | Zootoca vivipara | Common lizard |
| Reptile | APOBEC1 | Podarcis muralis | Wall lizard |
| Reptile | APOBEC1 | Alligator sinensis | Chinese aligator |
| Reptile | APOBEC1 | Anolis carolinensis | Green anole lizard |
| Amphibian | APOBEC1 | Gopherus evgoodei | Goodes thornscrub tortoise |
| Amphibian | APOBEC1 | Dermochelys coriacea | Leatherback sea turtle |
| Amphibian | APOBEC1 | Chrysemys picta bellii | Western painted turtle |

The following methods were used in this example for assessing on-targeting base editing and off-targeting in Jurkat cells.

### Assessment of On-Target Base Editing in Jurkat Cells

Wild type and engineered variants of APOBEC1s (Table 1) were incorporated into the BE architecture of an improved CBE described in Komor et al. 2017. CBE constructs were subcloned into and IVT vector and mRNA of each base editor produced. CBE mRNA and sgRNA were nucleofected into Jurkat E6.1 cells. Following 96 hours after nucleofection, cell pellets were isolated and genomic DNA was extracted for amplicon-seq of the targeted loci. For β2M and FAS, cells were immunostained for to assess protein knockdown by flow cytometry.

### Assessment of Off-Target DNA C→T Conversions in Jurkat Cells

Determination of Cas-independent off-target editing was performed using modified version of the previously described method where a catalytically impaired SaCas9 in combination with an sgRNA creates long-lived regions of ssDNA (R-loops) that can be subjected to deamination by an active SpCas9-CBE (Yu et al. 2020; Doman et al. 2020). A Jurkat cell line stably expressing SaCas9 (D10A)-2XUGI (Yu et al. 2020) was created by lentiviral transduction of the SaCas9 (D10A)-2XUGI gene fused to a P2A-Hyromycin resistance cassette (FIG. 16, top panel). Codon-optimized SaCas9 (D10A)-2XUGI-P2A-Hygromycin resistance expression construct was cloned into a third generation lentiviral vector and lentiviruses were produced. Jurkat cells were transduced with these lentiviruses and the transduced cells were selected using hygromycin. Following selection with hygromycin, pancellular expression of SaCas9 (D10A)-2XUGI was confirmed by flow cytometry. Off-target spurious deamination in the lenti-SaCas9 (D10A)-2XUGI cells was observed by just adding the CBE mRNAs and SaCas9 sgRNAs (FIG. 16, bottom panel) (Table 5). Table 5 confirms that the modified R-loop assay using the stable cell lines is capable of capturing the spurious off-target C to T conversions without the need of transfecting additional SaCas9 (D10A)-2XUGI mRNA. Table 5 also confirms that the modified R-loop assay shows similar spurious deamination trends as the previously described methods with the base editors listed in Table 1. This modified R-loop assay can avoid variability between the R-loop assay experiments as the entire bulk-cell population express nSaCas9 and the stable expression of nSaCas9 can amplify the off-target rates compared to transient transfection methods, leading to higher confidence in the deaminase selected using this method for low spurious deamination activity. To evaluate the off-target activity of the deaminases listed in Table 4, SpCas9 CBE mRNA and SaCas9 sgRNAs were nucleofected into Jurkat lenti-SaCas9 (D10A)-2XUGI cells and cell pellets harvested for DNA extraction 96 hours after nucleofection. Genomic DNA was extracted from cell pellets and sent for amplicon sequencing of the targeted R-loop regions. Percent C→T conversion for all cytosines in each amplicon was quantitated.

TABLE 5
Spurious off-target deamination by the modified R-loop Assay

| | Cells stably expressing | Transient transfection of |
|---|---|---|
| CBE | SaCas9(D10A)-2XUGI | SaCas9(D10A)-2XUGI mRNA |
| Rat | 24.10% | 12.83% |
| Rat-YE1(W90Y R126E) | 1.56% | 0.86% |
| Orangutan | 1.71% | 3.28% |
| Orangutan-R33A | 0.82% | 0.74% |
| Orangutan-H122A | 0.59% | 0.31% |
| Alligator | 0.47% | 0.23% |
| Rat-E63A | 1.12% | 0.23% |

Doman 6, SEQ ID NO: 86 (guide sequence-GATGTTCCAATCAGTACGCA) was used.

### Example 2

### Use of an Exemplary Cytosine Base Editor for Multiplex Editing of a CAR T Cell

The cytosine base editors described in Example 1 were tested in an exemplary CAR T cell to demonstrate efficiency multiplex editing.

The CAR T cell has edits in the TRAC, β2M, CD70, Regnase, and TGFBRII genes to knock out gene expression and additionally expresses an anti-CD70 CAR. The CAR T cell was generated either by CRISPR-Cas9 editing or by editing the genes by an exemplary cytosine base editor with an orangutan deaminase followed by incorporation of the CAR by AAV insertion. Table 6 describes the study design and Table 7 has details of the gRNAs used.

TABLE 6
Study design

| SAMPLE | CONDITION | EP1 | EP2 (RNP) |
|---|---|---|---|
| 1 | Unedited | NA | NA |
| 2 | 4X-No AAV (BE) | B2M1/CD70/TGFBR2/Reg | NA |
| 3 | 5X-CTX131 (RNP) | B2M1/TGFBR2/Reg | CD70/TRAC/AAV |
| 4 | 5X-CTX131 (BE) | B2M1/CD70/TGFBR2/Reg | TRAC/AAV |

TABLE 7
Guide details

| EDITING | | | Guide Sequence |
|---|---|---|---|
| TYPE | GENE | GUIDE NAME | (Target Seq) |
| Base Editing | TGFBR2 | pmSTOPTGFBR2Qexon5_T1 | ACTCCAGTTCCTGACGGCTGAGG |
| | | | (SEQ ID NO: 61) |
| | Regnase | pmSTOPREG1Exon1 | AATTCCCACAGACTCATGGT |
| | | | (SEQ ID NO: 62) |
| | CD70 | pmSTOPQCD70Exon1 | GTGCATCCAGCGCTTCGCAC |
| | | | (SEQ ID NO: 63) |
| | B2M1 | B2M-1/sdB2MEx1 | ACTCACGCTGGATAGCCTCC |
| | | | (SEQ ID NO: 64) |
| Conventional | TRAC | TRAC TA1 | AGAGCAACAGTGCTGTGGCC |
| RNP | | | (SEQ ID NO: 65) |
| | | | PAM: TGG |
| | TGFBR2 | TGFBRII_EX5_T1 | CCCCTACCATGACTTTATTC |
| | | | (SEQ ID NO: 66) |
| | | | PAM: TGG |
| | Regnase | ZC3H12A_segment-4_T7 | ACGACGCGTGGGTGGCAAGC |
| | | | (SEQ ID NO: 67) |
| | | | PAM: GGG |
| | CD70 | CD70 | GCTTTGGTCCCATTGGTCGC |
| | | | (SEQ ID NO: 68) |
| | | | PAM: GGG |
| | B2M1 | B2M-1 | GCTACTCTCTCTTTCTGGCC |
| | | | (SEQ ID NO: 69) |
| | | | PAM: TGG |

As shown in the table above, the conventional CRISPR-Cas9 editing took place in two electroporation events, wherein RNP nucleofection of β2M, Regnase, and TGFBRII guides took place in the first electroporation and RNP nucleofection of TRAC, CD70, and AAV insertion took place in the second electroporation event. In case of base editing, all 4 base editing guides were introduced into T cells during the first electroporation while AAV insertion into the TRAC locus took place in a second electroporation event by double stranded breaks and HDR using conventional CRISPR-Cas9 methods. An unedited control and base edited cells with no AAV insertion control were included in the study. The cytosine base editor (CBE) used comprised an orangutan APOBEC1 mRNA modified with N1-methyl-pseudouridine and has been described in the Example above. The CBE used was shown to have equivalent on-target activity as the rat APOBEC1 and has improved off-target activity compared to the rat APOBEC1 (see, for instance, FIG. 4).

### Assessment of Editing Efficiency

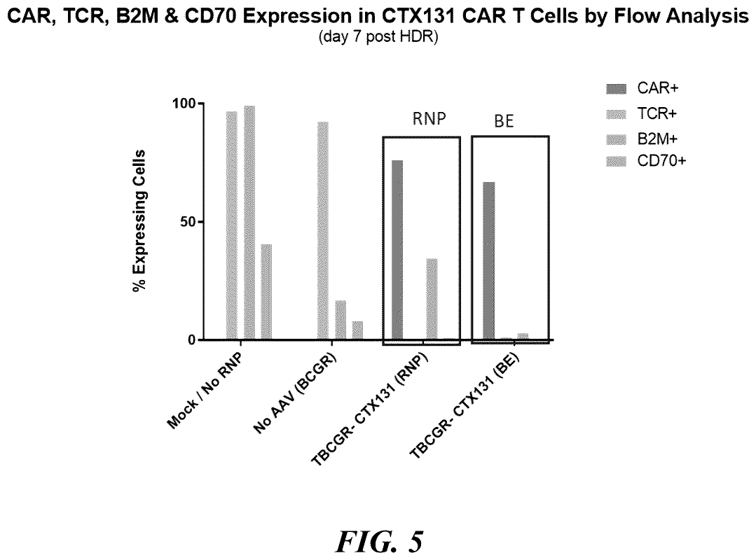

Editing efficiencies for CAR insertion, as well as TRAC, β2M and CD70 knockouts were measured on day 7 post-HDR. Flow cytometry results of gene expression levels are presented in FIG. 5 and indicate that CAR expression was similar (~70%) across all genotypes.

Editing efficiency for TRAC, β2M and CD70 knockouts demonstrated >98% knockout of TCR and CD70 expression. There was approximately 65% KO of 82M by RNP editing while base editing gave 97% knockout.

TABLE 8
% expressing cells

| | CAR+ | TCR+ | B2M+ | CD70+ |
|---|---|---|---|---|
| Mock/No RNP | 0.3 | 97.0 | 99.1 | 40.6 |
| No AAV | 0.1 | 92.5 | 16.9 | 8.1 |
| Edited cells (RNP) | 76.0 | 0.6 | 34.6 | 1.0 |
| Edited cells (BE) | 66.8 | 1.3 | 2.8 | 0.2 |

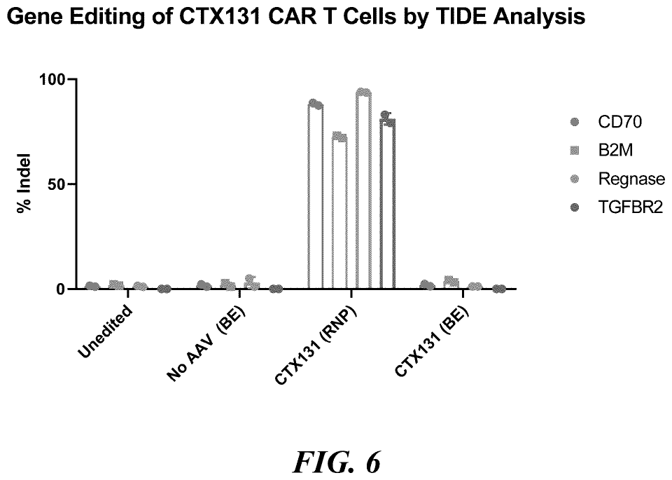

Assessment of indels in edited cells was also done by TIDE analysis. The results are presented in FIG. 6 and show the absence of indels in base edited CAR T cells.

### Assessment of CD4:CD8 Ratios

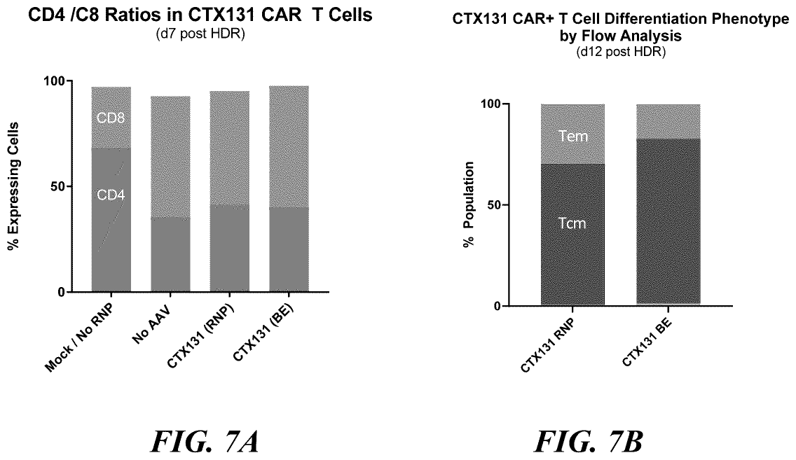

The frequency of CD4 and CD8 T cells in these cell cohorts, as well as differential profiles of the CAR T cells was also determined by flow cytometry. Average frequencies are enumerated in FIG. 7A and Table 9. There was no change in CD4+ to CD8+ cell ratios in any of the CAR T cell populations. All cell populations were highly enriched for central memory cells (FIG. 7B).

TABLE 9
% expressing cells

| | CD4+ | CD8+ |
|---|---|---|
| Mock/No RNP | 68 | 29 |
| BE/No AAV | 35 | 57 |
| Edited cells (RNP) | 41 | 54 |
| Edited cells (BE) | 40 | 58 |

### Assessment of T-Cell Expansion and Viability

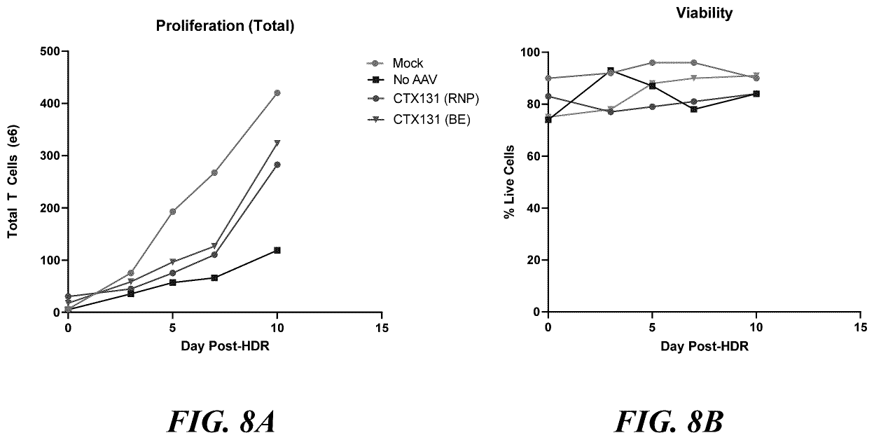

The cells were counted at regular intervals to assess T-cell expansion. The results are presented in FIG. 8A and Table 10 below. The data demonstrated equivalent proliferative capacity of base edited- and RNP-edited T cells. CAR T cell viability was not significantly different between cells edited with the CBE and cells edited via RNP (FIG. 8B).

TABLE 10
T-cell Number (×10⁶)

| Day | | | Edited | Edited |
|---|---|---|---|---|
| Post- | | BE/No | cells | cells |
| HDR | Mock | AAV | (RNP) | BE |
| 0 | 6 | 5 | 30 | 18 |
| 3 | 75 | 35 | 45 | 59 |
| 5 | 193 | 57 | 75 | 96 |
| 7 | 267 | 66 | 110 | 127 |
| 10 | 420 | 119 | 282 | 323 |

### Assessment of In Vitro Cytotoxicity

A cell killing (cytotoxicity) assay was used to assess the ability of the CAR⁺ T cells to cause cellular lysis in target cancer cells.

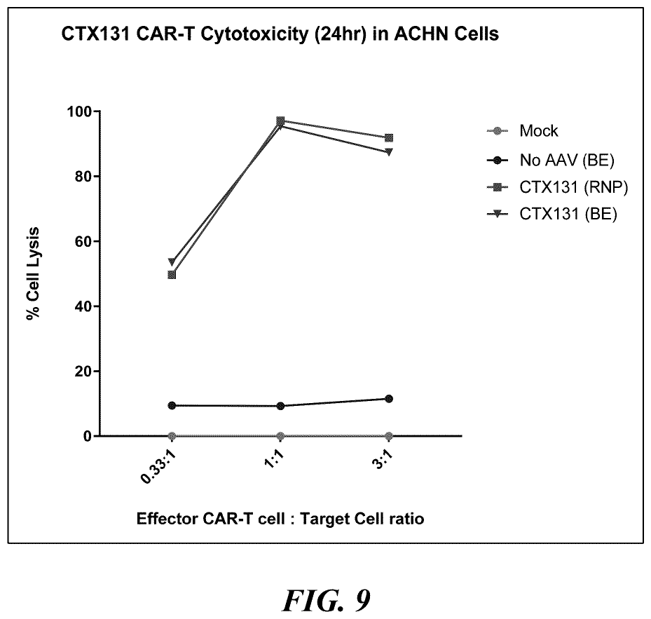

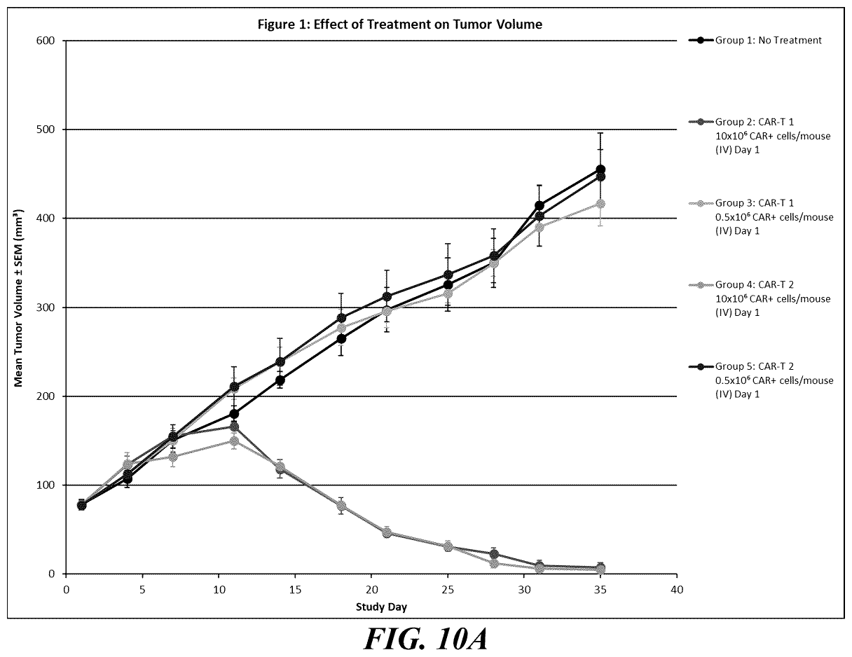

CAR T cells were plated at different ratios with ACHN target cells that are a low CD70-expressing RCC cell line and therefore, challenging to kill. One day later, the number of viable target cells was counted. Cell lysis results are presented in FIG. 9 and Table 11 below. The data demonstrates that CAR T cells edited using RNP or the base editor were equally efficacious in killing the target cells.

TABLE 11
% ACHN cell lysis

| T cell:target | BE/No | Edited | Edited |
|---|---|---|---|
| cell ratio | AAV | cells (RNP) | cells (BE) |
| 0.33:1 | 9 | 50 | 53 |
| 1:1 | 9 | 97 | 95 |
| 3:1 | 12 | 92 | 87 |

Cytokine secretion of the CAR T cells in the presence of ACHN target cells, which express CD70, was also measured. Secretion of IFN-gamma and IL-2 demonstrated functional CAR T cells generated by either RNP or base editor editing, as shown in Tables 12-13 below.

TABLE 12
IFN-gamma (pg/ml)

| T cell: target | | BE/No | Edited | Edited |
|---|---|---|---|---|
| cell ratio | Mock | AAV | cells (RNP) | cells (BE) |
| 3:1 | 758 | 24996 | 53593 | 53686 |
| 1:1 | 228 | 6573 | 57208 | 45850 |
| 0.3:1 | 0 | 1870 | 43184 | 44571 |

TABLE 13
IL-2 (pg/ml)

| T cell:target | | BE/No | Edited cells | Edited |
|---|---|---|---|---|
| cell ratio | Mock | AAV | (RNP) | cells (BE) |
| 3:1 | 8 | 19 | 1334 | 884 |
| 1:1 | 7 | 8 | 5825 | 4981 |
| 0.3:1 | −2 | 3 | 7079 | 7592 |

### Assessment of In Vivo Cytotoxicity

Efficacy of the CAR T cells in a Caki-1 renal cell carcinoma tumor xenograft model in NSG mice was evaluated.

NSG mice were subcutaneously implanted on the right flank with 5×10⁶ tumor cells in 50% Matrigel/50% media. On day 1, the mice were randomized into 5 groups and injected intravenously with CAR T cells as described in Table 14.

TABLE 14
In vivo tumor study groups

| | | CAR T | CAR T cell | CAR T | CAR T |
|---|---|---|---|---|---|
| | | cell (RNP | (RNP | cell (BE | cell (BE |
| Group | Number | edited) | edited) | edited) | edited) |
| No treatment | 5 | | | | |
| 10 × 10⁶ cells/mouse | 5 | X | | | |
| 0.5 × 10⁶ cells/mouse | 5 | | X | | |
| 10 × 10⁶ cells/mouse | 5 | | | X | |
| 0.5 × 10⁶ cells/mouse | 5 | | | | X |

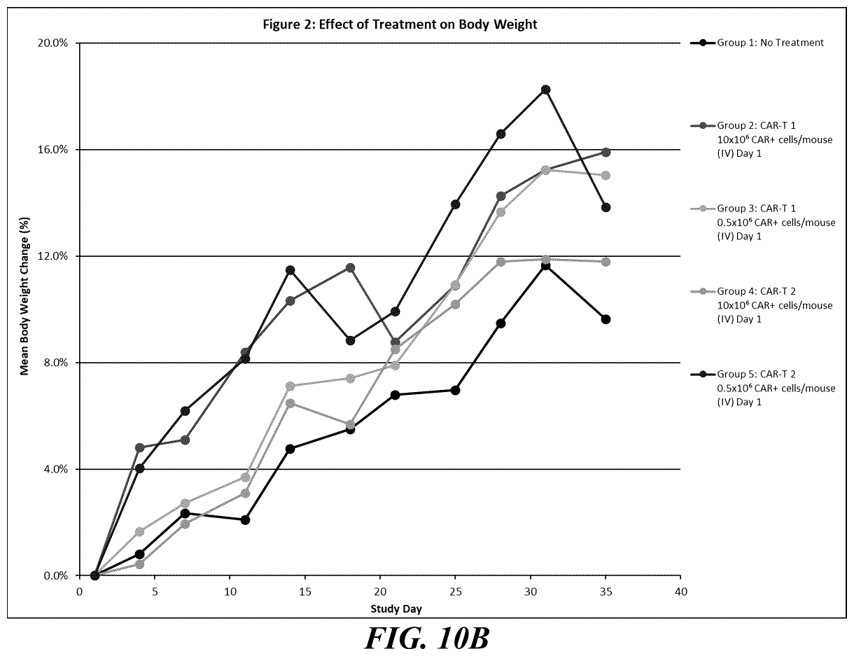

Tumor volumes were evaluated every few days. Tumor volumes are presented in FIG. 10A and Table 15. As shown, there was significant reductions in tumor volumes in mice treated with 10×10⁶ CAR T cells that were either RNP-edited or CBE-edited. There was no significant weight loss in animals treated with CAR T cell therapy (FIG. 10B)

TABLE 15
Mean tumor Volume (mm³)

| | Day 1 | Day 4 | Day 11 | Day 14 | Day 18 | Day 21 | Day 28 | Day 35 |
|---|---|---|---|---|---|---|---|---|
| No treatment | 78 | 107 | 180 | 219 | 265 | 297 | 350 | 456 |
| 10 × 10⁶ RNP- | 78 | 123 | 166 | 118 | 77 | 46 | 23 | 8 |
| edited cells | | | | | | | | |
| 0.5 × 10⁶ RNP- | 77 | 113 | 208 | 238 | 277 | 296 | 350 | 417 |
| edited cells | | | | | | | | |
| 10 × 10⁶ BE- | 78 | 124 | 150 | 121 | 77 | 47 | 12 | 4 |
| edited cells | | | | | | | | |
| 0.5 × 10⁶ BE- | 78 | 113 | 211 | 239 | 288 | 313 | 358 | 447 |
| edited cells | | | | | | | | |

### Assessment of Multiplex Editing Efficiency

Multiplex base editing efficiency for various combinations of target gene knockout was measured. Editing efficiency of multiple base editing is presented in Table 16 and 17.

TABLE 16
Editing efficiency of multiplex base
editing via genomic (ICE) analysis

| Groups | | | % BE | % Base |
|---|---|---|---|---|
| (% | | | in a | editing |
| Editing | | | single | in multi- |
| eff) | Locus | Guide | edit | plexing |
| M1 | FAS | saFASExon4 | 76 | 64 |
| (59%) | TRAC | saTRACExon3 | 100 | 99 |
| | B2M | sdB2MExon 1 | 98 | 97 |
| | CD70 | sdCD70Exon1 | 96 | 97 |
| M2 | REGI | pmSTOPREGIExon1 | 98 | 99 |
| (91%) | TRAC | saTRACExon3 | 100 | 99 |
| | B2M | sdB2MExon1 | 98 | 98 |
| | CD70 | sdCD70Exon1 | 96 | 95 |
| M3 | TGFBRII | pmSTOPQTGFBRIIExon5 | 95 | 93 |
| (85%) | B2M | sdB2MExon1 | 98 | 96 |
| | CD70 | sdCD70Exon1 | 96 | 97 |
| | REGI | pmSTOPReg1Exon1 | 98 | 98 |
| M4 | FAS | saFASExon4 | 76 | 66 |
| (58%) | TRAC | saTRACExon3 | 100 | 100 |
| | B2M | sdB2MExon1 | 98 | 95 |
| | CD70 | sdCD70Exon1 | 96 | 96 |
| | REGI | pmSTOPREGIExon1 | 98 | 96 |

TABLE 17
4-plex editing in primary T cells with a CBE

| | | % C to T | % C to T | % C to T |
|---|---|---|---|---|
| | | conversion | conversion | conversion |
| GROUP | Locus | in a single edit | in 4-plex edit | over all |
| Group 1 | TRAC | 100 | 99 | 91% |
| | B2M | 98 | 98 | |
| | CD70 | 96 | 95 | |
| | Regnase-1 | 98 | 99 | |
| Group 2* | B2M | 98 | 96 | 85% |
| | CD70 | 96 | 97 | |
| | Regnase-1 | 98 | 98 | |
| | TGFBR2 | 95 | 93 | |

Group 2 demonstrates the same knockout profile as CTX131.

The data in the above table demonstrates that base editing is stochastic in a population of cells. In other words, the fraction of cells with all edits equals the product of the frequencies of individual edits.

Optimization of BE4 mRNA and sgRNA for Multiplexing

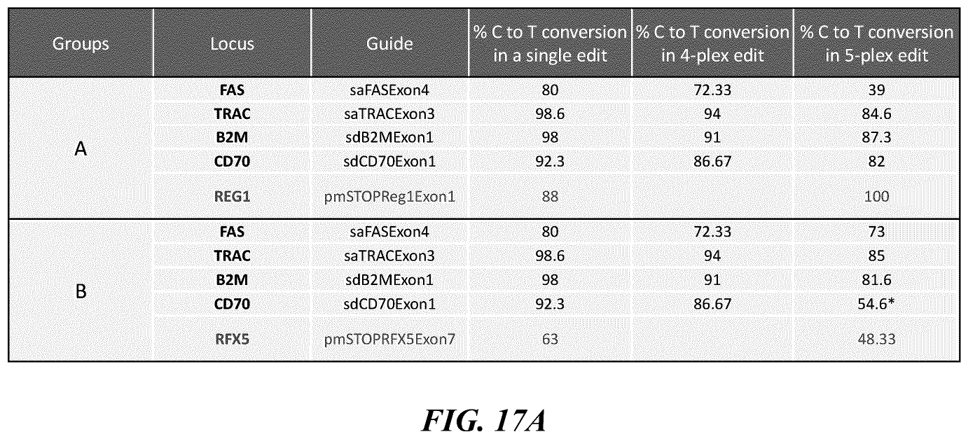

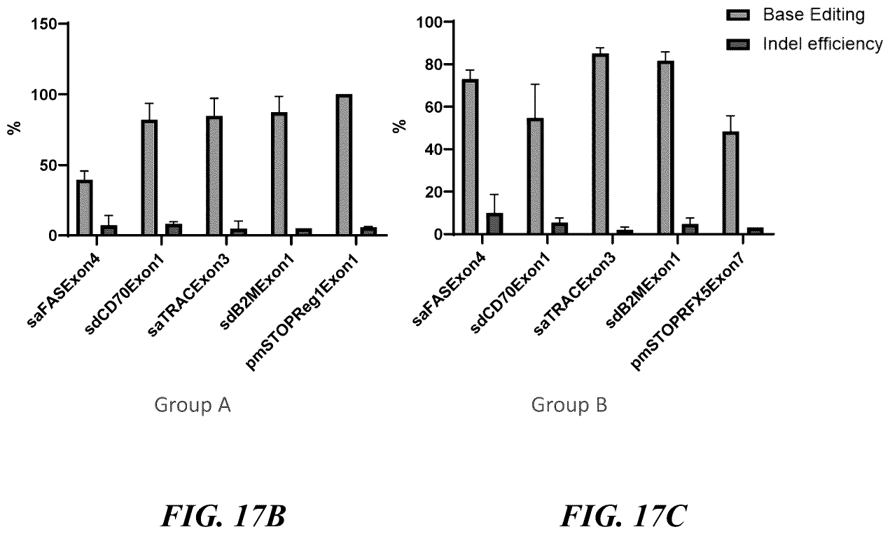

Editing efficiencies for Group A (FAS, TRAC, B2M, CD70, and optionally REG1) and Group B (FAS, TRAC, B2M, CD70, and optionally RFX5) are presented in FIG. 17A and graphically represented in FIGS. 17B-C. The data indicates a visible drop in editing efficiency as the number of targets increases (from 4 targets to 5 targets). A slight decrease per guide can possibly lead to a dramatic loss of overall editing efficiency.

To optimize multiplex editing, various formulations and doses of mRNA and sgRNA were evaluated. Table 18 shows an exemplary mRNA and guide RNA formulation. The amount in Table. 18 are for triplicate sample sets. The data demonstrates a minimal amount of BE mRNA of 1.15 μmol/million T cells and amount of sufficient guide (93.2 μmol of each sgRNA per 1 million Tcells) required for multiplex editing.

TABLE 18
An exemplary formulation of BE mRNA and guide RNA

| | mRNA (μL | | Guide per |
|---|---|---|---|
| | equivalent | | target locus |
| | of 1.15 | Total | (μL equivalent |
| | pmol/ | amount of | of 93.2 |
| Groups | sample) | guide (μL) | pmol/sample) |
| saFASExon4 + sdCD70 | 8.5 | 7.215483871 | 1.80 per guide |
| Exon1 + TRACEx3SA + | | | |
| B2MEx1 SD | | | |
| saFASExon4 + sdCD70 | 8.5 | 9.019354839 | 1.80 per guide |
| Exon1 + TRACEx3SA + | | | |
| B2MEx1 SD + | | | |
| pmSTOPReg1Exon1 | | | |
| saFASExon4 + sdCD70 | 8.5 | 9.019354839 | 1.80 per guide |
| Exon1 + TRACEx3SA + | | | |
| B2MEx1SD + | | | |
| pmSTOPRFX5Exon7 | | | |

Formulation and dose of mRNA are also optimized to find minimal amount of mRNA to guide ratio. Experiment course can be timed to study the duration of BE4 expression. An exemplary formulation and dose of mRNA are presented in Table 19. Excessive mRNA (e.g., 6 μg) can result in a drop in cell viability.

TABLE 19
An exemplary formulation and dose of mRNA

| GUIDES | mRNA (μg) |
|---|---|
| sdFASEx7 | 1.5 |
| sdCD70Ex1 | 1.5 |
| TRACEx3 SA | 1.5 |
| B2MEx1 SD | 1.5 |
| FAS + CD70 + B2M + TRAC | 3 |
| FAS + CD70 + B2M + TRAC | 6 |
| FAS + CD70 + B2M + TRAC + REG1 | 3 |
| FAS + CD70 + B2M + TRAC + RFX5 | 3 |

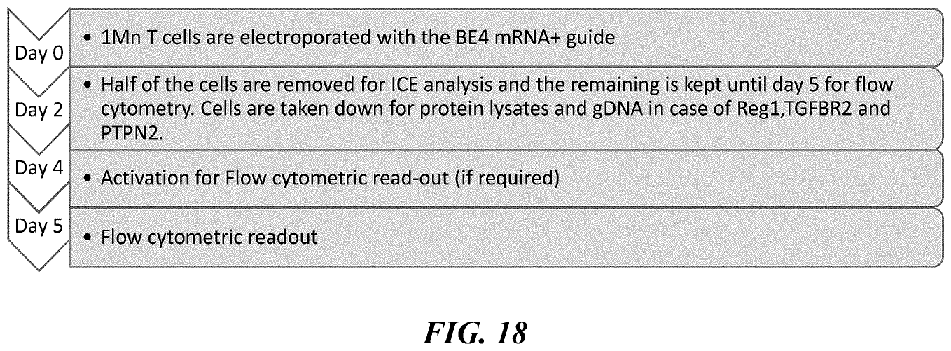

FIG. 18 illustrates an experiment layout in an exemplary embodiment. 1Mn T cells are electroporated with the BE4 mRNA and guide RNA. at Day 1, half of the cells are removed for ICE analysis and the remaining is kept until day 5 for flow cytometry. Cells are taken down for protein lysates and gRNA in case of Reg1, TGFBR2 and PTPN2. At Day 4, cells are activated for flow cytometric readout if required. At Day 5, flow cytometry results of gene expression levels are obtained. All the experiments are conducted with triplicate samples per guide. Table 20 below presents an exemplary flow cytometry and ICE analysis read-out.

TABLE 20
Flow cytometry and ICE analysis read-out.

| | BE4 mRNA | sgRNA | B2M | TRAC | FAS | CD70 |
|---|---|---|---|---|---|---|
| Sample | (pmol/Mn | (pmol/Mn | (% protein | (% protein | (% protein | (% Base |
| set | cells) | cells) | knockdown) | knockdown) | knockdown) | Editing ICE) |
| S1 | 1.15 | 93.2 | 96 | 99 | 73.7 | 81 |
| S2 | 0.575 | | 96 | 98 | 65.3 | 91 |
| S3 | 0.287 | | 94 | 97 | 52.9 | 92 |
| S4 | 0.143 | | 91 | 96 | 33.0 | 94 |
| | sgRNA | BE4 mRNA | B2M | TRAC | FAS | CD70 |
| Sample | (pmol/Mn | (pmol/Mn | (% protein | (% protein | (% protein | (% Base |
| set | cells) | cells) | knockdown) | knockdown) | knockdown) | Editing ICE) |
| S1 | 93.2 | 1.15 | 97 | 99 | 76.9 | 94 |
| S2 | 46.6 | | 96 | 98 | 63.2 | 95 |
| S3 | 23.3 | | 96 | 98 | 45.5 | 94 |
| S4 | 11.65 | | 94 | 98 | 25.0 | 94 |

In the first set of samples (S1-S4), sgRNA of 93.2 μmol per million cells was used together with different amounts of BE mRNA. In the second set of samples (S1-S4), BE4 mRNA of 1.15 μmol per million cells was used together with different amounts of sgRNA. B2M, TRAC and FAS knockout rates and CD70 base editing rate were evaluated for each sample set. In both conditions, TRAC, B2M and CD70 guides are agnostic to titration while the FAS guide follows a dose-dependent decrease.

### Example 3

### Use of an Exemplary Cytosine Base Editor for Targeted KO of an Apo(a) Gene

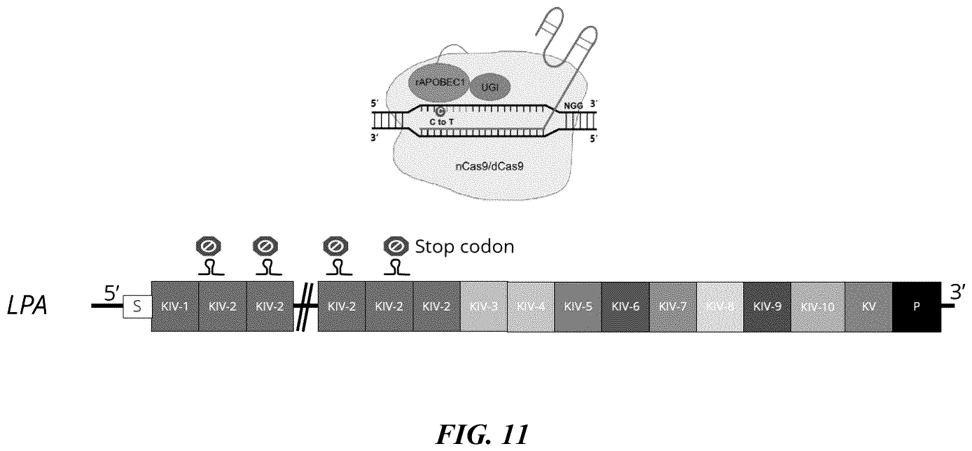

The use of the cytosine base editor described in Example 1 was tested for targeted knockdown of an Apo(a) gene (apolipoprotein A component of Lp(a)) that would lead to permanent reduction in Lp(a) levels to normal or low normal range (for example 125 nmol/L). sgRNAs were designed to target regions in the Kringle IV-2 repeats of the LPA gene (FIG. 11) to introduce stop codons. As described herein, efficient knockdown was achieved in human cultured cells and reduction in serum Lp(a) in NHPs were also observed. Non-limiting exemplary gRNAs for LPA gene are provided in Table 21.

TABLE 21
Guide RNAs for LPA gene

| Guide RNA Name | Target | Sequence | PAM |
|---|---|---|---|
| pmSTOPLPA Exon 3b | Exon 3 | ACGCAATGCTCAGACGCAGA | AGG |
| | | (SEQ ID NO: 70) | |
| pmSTOPLPSQ Exon 3 | Exon 3 | CGAACAAGGTAAGGAGTCTG | TGG |
| | | (SEQ ID NO: 71) | |
| pmSTOPQLPA Exon 4a | Exon 4 | TGAGCAAAGGCCTGGGGTGC | AGG |
| | | (SEQ ID NO: 72) | |
| pmSTOPQLPA Exon 4b | Exon 4 | GGGGTGCAGGAGTGCTACCA | TGG |
| | | (SEQ ID NO: 73) | |
| pmSTOPQLPA Exon 4c | Exon 4 | GGTAATGGACAGAGTTATCG | AGG |
| | | (SEQ ID NO: 74) | |
| pmSTOPWLPA Exon 4 | Exon 4 | GAATACTACCCAAATGCGTA | TGG |
| | | (SEQ ID NO: 75) | |
| saLPA Exon 4a | Exon 4 | GGATCCCTCGTATAACAATA | AGG |
| | | (SEQ ID NO: 76) | |
| saLPA Exon 4b | Exon 4 | TGGACTACATAGTTGTGTGA | GGG |
| | | (SEQ ID NO: 77) | |
| saLPA Exon 4 | Exon 4 | TTCAATCCTCTTGTCACCTG | TGG |
| | | (SEQ ID NO: 78) | |
| LPA_CDS_V01_ | Exon 3 | CGAACAAGGTAAGGAGCCTG | TGG |
| (pmSTOP E3_NHP | | (SEQ ID NO: 79) | |
| Matched) | | | |
| LPA_CDS_G697 | Exon 3 | TCGCCCTTGGTGTTACACCA | TGG |
| | | (SEQ ID NO: 80) | |
| LPA_CDS_G303 | Exon 3 | TCGCCCTTGGTGTTATACCA | TGG |
| | | (SEQ ID NO: 81) | |
| LPA_CDS_G167 | Exon 3 | TGGTAGCAATCCTGGACCAC | AGG |
| | | (SEQ ID NO: 82) | |
| LPA_CDS_G532 | Exon 3 | CTGCCAAGCTTGGTCATCTA | TGG |
| | | (SEQ ID NO: 83) | |

### Base Editing in Human Liver Cell Lines

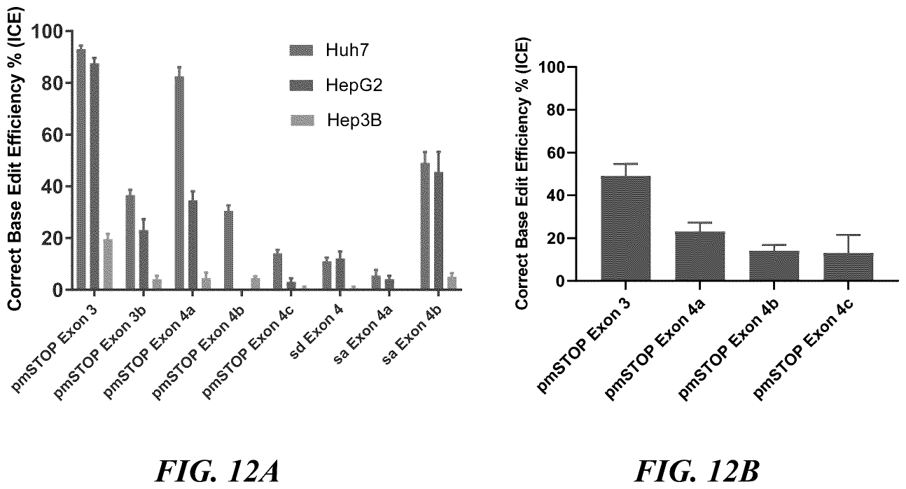

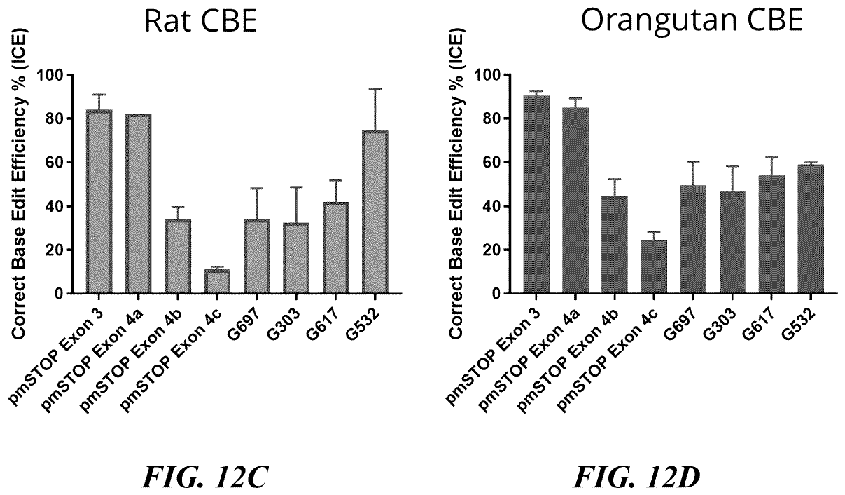

CBE mRNA comprising rat APOBEC1 and gRNA were transfected using Lipofectamine into human liver cell lines, Huh7, HepG2, and Hep3B, as well as iPSC-derived hepatoblasts. Base editing efficiency was measured. The results are presented in FIGS. 12A-B and Tables 22-23. ‘pmSTOP’ indicates a premature stop codon; ‘sa/sd’ indicates splice acceptor/donor defect.

TABLE 22
Base editing efficiency (%) in liver cell lines

| gRNA | HepG2 | Hep3B | Huh7 |
|---|---|---|---|
| pmSTOP Exon 3 | 88 | 20 | 93 |
| pmSTOP Exon 3b | 23 | 4 | 37 |
| pmSTOP Exon 4a | 35 | 5 | 83 |
| pmSTOP Exon 4b | n.d. | 5 | 31 |
| pmSTOP Exon 4c | 3 | 1 | 14 |
| sd Exon 4 | 12 | 1 | 11 |
| sa Exon 4a | 4 | 0 | 6 |
| sa Exon 4b | 46 | 5 | 49 |

TABLE 23
Base editing efficiency (%) in iPSC-derived hepatoblasts

| gRNA | % |
|---|---|
| pmSTOP Exon 3 | 49 |
| pmSTOP Exon 4a | 23 |
| pmSTOP Exon 4b | 14 |
| pmSTOP Exon 4c | 13 |

### Comparison of Base Editors in a Human Liver Cell Line

CBE mRNA comprising rat APOBEC1 deaminase or orangutan deaminase (described above) and gRNA were transfected using Lipofectamine into Huh7. Base editing efficiency was measured. The results are presented in FIGS. 12A-D and Table 24.

TABLE 24
Base editing efficiency (%) in Huh7 cells

| | Rat CBE | Orangutan CBE |
|---|---|---|
| pmSTOP Exon 3 | 84 | 91 |
| pmSTOP Exon 4a | 82 | 85 |
| pmSTOP Exon 4b | 34 | 45 |
| pmSTOP Exon 4c | 11 | 25 |
| G697 | 34 | 50 |
| G303 | 33 | 47 |
| G617 | 42 | 55 |
| G532 | 75 | 59 |

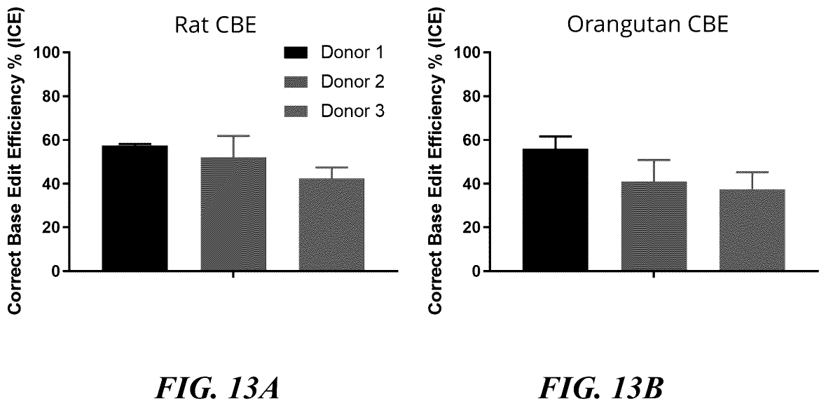

Rat CBE and Orangutan CBE were also compared to NHP primary liver cells from 3 donors. The results are presented in FIGS. 13A-B and Table 25.

TABLE 25
Base editing efficiency (%) in NHP primary liver cells

| | Rat CBE | Orangutan CBE |
|---|---|---|
| Donor 1 | 58 | 41 |
| Donor 2 | 43 | 38 |
| Donor 3 | 52 | 56 |

Overall, these studies have identified a high efficiency orangutan CBE-guide gRNA for gene editing of an LPA gene. The orangutan CBE performs at similar efficiency as the rat APOBEC1 CBE.

### Example 4

### Multi-Plex Base Editing in T Cells: Orthologue Comparison

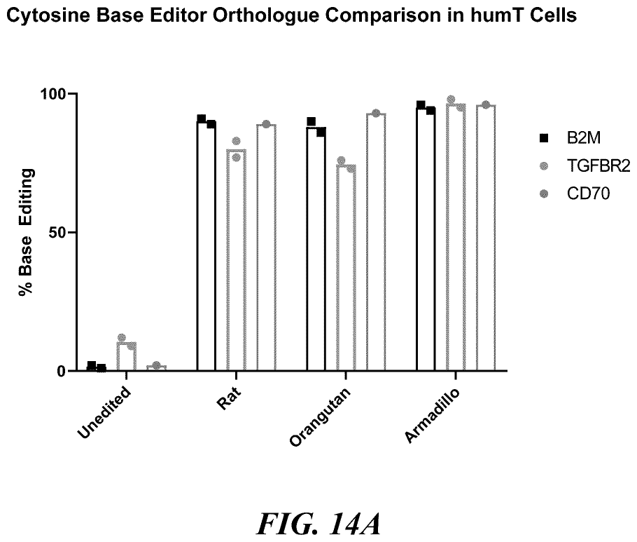

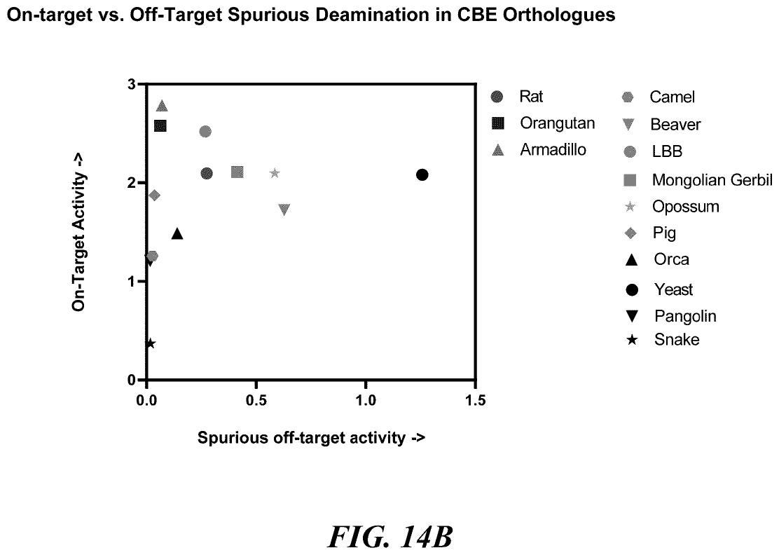

Cytosine base editors comprising rat, Orangutan and Armadillo deaminase, respectively, were used in multi-plex base editing in T cells. Comparison of the three deaminases in % base editing and on-target vs. off-target activity are shown in FIGS. 14A-14B. FIG. 14A shows % base editing (C>T conversion), demonstrating slightly improved multi-plex base editing for Armadillo CBE across 3 loci in T cells. FIG. 14B shows that Armadillo CBE performed the best among the three deaminases tested in on-target vs. off-target spurious deamination, and Orangutan deaminase performed better than rat deaminase. Shown in FIG. 14B, off-target activity was determined as the sum of C>T rates across R loop amplicons, and on-target activity was determined as the sum of C>T rates across on-target spacer regions.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A fusion protein comprising:
a Cas9 domain, wherein the Cas9 domain when associated with a guide RNA (gRNA) specifically binds to a target nucleic acid sequence; and
a cytidine deaminase domain capable of deaminating a cytosine base in a single-stranded portion of the target nucleic acid sequence, wherein the cytidine deaminase domain comprises a deaminase from an apolipoprotein B mRNA-editing complex (APOBEC) family deaminase.

2. The fusion protein of claim 1, wherein the APOBEC family deaminase is an APOBEC1 deaminase.

3. The fusion protein of any one of claims 1-2, wherein the cytidine deaminase domain comprises a mammalian deaminase or a variant thereof.

4. The fusion protein of any one of claims 1-3, wherein the cytidine deaminase domain comprises a rat deaminase, an armadillo deaminase, a bat deaminase, or a variant thereof.

5. The fusion protein of any one of claims 1-4, wherein the cytidine deaminase domain comprises a deaminase from Dasypus novemcinctus, Meriones unguiculatus, Myotis lucifugus, or a variant thereof.

6. The fusion protein of any one of claims 1-5, wherein the cytidine deaminase domain comprises (1) an amino acid sequence having at least 85%, 90%, 95%, 98%, or 99% identity to any one of SEQ ID NOs: 1-18, or (2) an amino acid sequence having one, two, three, four, five, six, seven, eight, nine, or ten mismatches relative to any one of SEQ ID NOs: 1-18.

7. The fusion protein of any one of claims 1-6, wherein the cytidine deaminase domain comprises amino acid mutation(s) at one or more positions functionally equivalent to R30, E31, L32, R33, K34, E35, T36, R52, Q56, N57, N59, K60, H61, V62, L88, S89, W90, R118, Y120, H121, H122, R126, R128, R169, 1195, R197, R198, K199, Q200, P201, Q202, and L203 in the deaminase of SEQ ID NO: 1.

8. The fusion protein of claim 7, wherein the amino acid mutation(s) comprise one or more amino acid substitutions of R33A, K34A, W90Y, H126E and H122A.

9. The fusion protein of any one of claims 1-6, wherein the cytidine deaminase domain does not comprise amino acid mutation(s) at one or more positions functionally equivalent to R33, K34, T36, R52, H53, Q56, K60, V62, R118, R126, R128, R169, and R198 in the deaminase of SEQ ID NO: 1.

10. The fusion protein of any one of claims 1-5, wherein the cytidine deaminase comprises an amino acid sequence selected from the group consisting of SEQ ID NOs: 1-18.

11. The fusion protein of any one of claims 1-10, wherein the cytidine deaminase is encoded by (1) a nucleic acid sequence having at least 85%, 90%, 95%, 98%, or 99% identity to any one of SEQ ID NOs: 19-36; or (2) a nucleic acid sequence having one, two, three, four, five, six, seven, eight, nine, or ten mismatches relative to any one of SEQ ID NOs: 19-36.

12. The fusion protein of any one of claims 1-10, wherein the cytidine deaminase is encoded by a nucleic acid sequence selected from the group consisting of SEQ ID NOs: 19-36.

13. The fusion protein of any one of claims 1-12, wherein the cytidine deaminase domain is fused to the N-terminus or C-terminus of the Cas9 domain.

14. The fusion protein of any one of claims 1-13, wherein the Cas9 domain is fused to the cytidine deaminase domain directly or via a linker.

15. The fusion protein of any one of claims 1-14, wherein the Cas9 domain of is a nuclease-inactive Cas9, a dead Cas9, a Cas9 nickase, or a fragment or a variant thereof.

16. The fusion protein of any one of claims 1-15, wherein the Cas9 domain is Streptococcus pyogenes (SpyCas9), Staphylococcus lugdunensis (SluCas9), or derived from Staphylococcus aureus (SaCas9), Neisseria meningitides Cas9, Streptococcus thermophilus Cas9, Treponema denticola Cas9, Campylobacter jejuni Cas9, S. schleiferi Cas9, or a variant thereof.

17. The fusion protein of any one of claims 1-16, further comprising an uracil glycosylase inhibitor (UGI) domain, wherein the UGI domain inhibits a uracil-DNA glycosylase.

18. The fusion protein of claim 17, wherein the UGI domain is fused to the N-terminus or C-terminus of the Cas9 domain.

19. The fusion protein of claim 18, wherein the UGI domain is fused to the N-terminus or C-terminus of the deaminase domain.

20. The fusion protein of any one of claims 17-19, wherein the UGI domain is fused to the deaminase domain and/or the Cas9 domain directly or via a linker.

21. The fusion protein of any one of claims 17-20, comprising two UGI domains.

22. The fusion protein of any one of claims 1-21, further comprising a nuclear localization sequence.

23. A nucleic acid sequence comprising a sequence encoding the fusion protein of any one of claims 1-22.

24. The nucleic acid sequence of claim 23, comprising a sequence encoding the cytidine deaminase domain having at least 85%, 90%, 95%, 98%, or 99% identity to any one of SEQ ID NOs: 19-36.

25. A complex comprising the fusion protein of any one of claims 1-22 and a guide RNA bound to the Cas9 domain of the fusion protein.

26. The complex of claim 25, wherein the gRNA is a single-guide RNA (sgRNA).

27. The complex of claim 25 or 26, wherein gRNA comprises a space sequence of any one of SEQ ID NOs: 61-86.

28. A composition, comprising:
(a) the fusion protein of any one of claims 1-22 or a nucleic acid sequence encoding the fusion protein of claim 23 or 24; and
(b) one or more guide RNAs targeting one or more target genes.

29. A pharmaceutical composition, comprising the composition of claim 28 and one or more pharmaceutical acceptable carriers or excipients.

30. A method for producing an engineered cell, comprising providing a plurality of cells;
delivering to the plurality of cells (a) a fusion protein of any one or claims 1-22 or a nucleic acid encoding the fusion protein and (b) at least one guide RNA targeting at least one target gene;
genetically editing the at least one target gene; and
producing one or more genetically engineered cells having at least one gene edit in the at least one target gene.

31. The method of claim 30, wherein the plurality of cells are T cells or precursor cells thereof.

32. The method of claim 31, comprising delivering to the plurality of cells a nucleic acid encoding a chimeric antigen receptor (CAR).

33. The method of any one of claims 30-32, wherein the at least one guide RNA comprises two or more guide RNAs targeting two or more target genes and wherein the produced one or more genetically engineered cells have two or more gene edits in the two or more target genes.

34. The method of any one of claims 30-33, wherein the at least one target gene is selected from the group consisting of the Regnase-1 (Reg1) gene, the Transforming Growth Factor Beta Receptor II (TGFBRII) gene, the TRAC gene, the beta-2-microglobulin (62M) gene, the CD70 gene, T cell receptor alpha chain constant region (TRAC) gene, or a combination thereof.

35. A population of genetically engineered cells, which is prepared by the method of any one of claims 30-34.

36. The population of genetically engineered cells of claim 35, comprising at least two gene edits.

37. The population of genetically engineered cells of claim 35 or 36, wherein at least 50% of the cells in the population of genetically engineered cells comprise at least two genome edits and wherein fewer than 1%, 0.5%, 0.2% or 0.1% of the cells in the population of genetically engineered cells have an insertion, deletion, translocation or other DNA rearrangement.

38. The population of genetically engineered cells of any one of claims 35-37, wherein the cells are T cells.

39. A method, comprising administering to a subject the population of genetically engineered cells of any one of claims 35-38.

40. The method of claim 39, wherein the subject is a human subject.

41. The method of claim 39 or 40, wherein the subject has a disease or disorder.

42. A method of DNA editing, comprising contacting a DNA molecule with the fusion protein of any one of claims 1-22; and
a guide RNA (gRNA) targeting a target nucleotide sequence of the DNA molecule, thereby editing the DNA molecule by deaminating a nucleotide base within the target nucleotide sequence of the DNA molecule.

43. The method of claim 42, wherein the target nucleotide sequence of the DNA molecule is associated with the disease or disorder.

44. The method of claim 43, wherein the deamination corrects a point mutation in the target nucleotide sequence associated with the disease or disorder.

45. The method of any one of claims 42-44, wherein the target nucleotide sequence encodes a protein and wherein the deamination results in a reduction or inhibition of the expression level of the encoded protein.

46. The method of any one of claims 42-45, wherein the target nucleotide sequence comprises a T to C point mutation associated with a disease or disorder and wherein the deamination of the mutant C base results in a sequence that is not associated with the disease or disorder.

47. The method of any one of claims 42-46, wherein the gRNA targets a target nucleotide sequence of a LPA gene.

48. The method of any one of claims 42-47, wherein the contacting is in vivo in a subject suspected to have, having or diagnosed with the disease or disorder.

49. A method of preventing or treating a disease or disorder in a subject in need thereof, comprising:
administering to the subject a therapeutically effective amount of the composition of claim 28 or the pharmaceutical composition of claim 29, wherein the one or more target gene is associated with the disease or disorder.

50. The method of any one of claims 41-49, wherein the disease or disorder is cancer.

51. The method of claim 50, wherein the cancer is selected from the group consisting of: pancreatic cancer, gastric cancer, ovarian cancer, uterine cancer, breast cancer, prostate cancer, testicular cancer, thyroid cancer, nasopharyngeal cancer, non-small cell lung (NSCLC), glioblastoma, neuronal, soft tissue sarcomas, leukemia, lymphoma, melanoma, colon cancer, colon adenocarcinoma, brain glioblastoma, hepatocellular carcinoma, liver hepatocholangiocarcinoma, osteosarcoma, gastric cancer, esophagus squamous cell carcinoma, advanced stage pancreas cancer, lung adenocarcinoma, lung squamous cell carcinoma, lung small cell cancer, renal carcinoma, intrahepatic biliary cancer, and a combination thereof.

52. The method of any one of claims 41-49, wherein the disease or disorder is calcific aortic valve disease, myocardial infarctions, coronary heart disease, atherosclerosis, thrombosis, stroke, coronary artery disease, familial hyperlipidemia, myocardial infarction, peripheral arterial disease, calcific aortic valve stenosis, or a combination thereof.

53. The method of any one of claims 41-49, wherein the disease or disorder is a metabolic disease, a lipid metabolism disease, obesity, atherosclerosis, hyperfattyacidemia, metabolic syndrome, dyslipidemia, hypobetalipoproteinemia, familial hypercholesterolemia (including homozygous familial hypercholesterolemia (HoFH) and heterozygous familial hypercholesterolemia (HeFH)), hypertriglyceridemia, familial combined hyperlipidemia, familial chylomicronemia syndrome, multifactorial chylomicronemia syndrome, familial combined hyperlipidemia (FCHL), metabolic syndrome (MetS), nonalcoholic fatty liver disease (NAFLD), elevated lipoprotein (a), cholesterol, and/or triglyceride in the blood, or a combination thereof.

54. The method of any one of claims 49 to 53, wherein the one or more target gene comprises a LPA gene.

55. The method of any one of claims 49 to 54, wherein the one or more target gene comprises an ANGPTL3 gene.

56. The method of any one of claims 49 to 55, wherein the one or more target gene comprise the Reg1 gene, the TGFBRII gene, the TRAC gene, the 82M gene, the TRAC gene, or a combination thereof.

57. A method, comprising:
transfecting a cell expressing an first Cas9 domain or a nucleic acid encoding the first Cas9 domain with a first guide RNA and a fusion protein comprising: (1) a second Cas9 domain, wherein the second Cas9 domain when associated with a second guide RNA specifically binds to a target nucleic acid sequence and (2) a cytidine deaminase domain capable of deaminating a cytosine base in a single-stranded portion of the target nucleic acid sequence, or a nucleic acid sequence encoding the fusion protein, wherein the first guide RNA when associated with the first Cas9 domain binds to a non-target nucleic acid sequence; and
analyzing the genomic sequence of the cell.

58. The method of claim 57, comprising producing the cell expressing the first Cas9 domain or the nucleic acid encoding the first Cas9 domain by transfecting the cell with a nucleic acid encoding the first Cas9 domain.

59. The method of any one of claims 57-58, wherein the first Cas9 domain comprises a Cas9 nickase, optionally a SaCas9 nickase.

60. The method of any one of claims 57-59, wherein the first Cas9 domain comprises two UGI domains.

61. The method of any one of claims 57-60, wherein the first Cas9 domain comprises a SaCas9 nickase and the second Cas9 domain comprises a SpyCas9 domain.

62. The method of any one of claims 57-61, wherein the first guide RNA is different from the second guide RNA.
